(19) United States
(12) Patent Application Publication
(10) Pub. No.: US 2009/0087417 A1
(43) Pub. Date: Apr. 2, 2009

(54) METHODS FOR PROMOTING DOPAMINERGIC NEURONAL DEVELOPMENT BY USING NG4A-SUBFAMILY AND WNT-LIGANDS

(76) Inventors: Ernesto Arenas, Stockholm (SE); Joseph Wagner, West Chester, PA (US); Goncalo Castelo Branco, Stockholm (SE); Kyle Sousa, Stockholm (SE)

Correspondence Address: MORRISON & FOERSTER LLP, 755 PAGE MILL RD, PALO ALTO, CA, 94304-1018, US

(21) Appl. No.: 12/147,420

(22) Filed: Jun. 26, 2008

Related U.S. Application Data
(63) Continuation of application No. 10/529,097, filed on Nov. 17, 2005.
(60) Provisional application No. 60/413,046, filed on Sep. 24, 2002.
(60) Provisional application No. 60/494,595, filed on Aug. 12, 2003.

(51) Int. Cl. A61K 35/30; C12N 5/10; A61K 35/12; A61P 25/00; C12Q 1/02; C12N 15/85

(52) U.S. Cl. 424/93.21; 435/377; 435/373; 435/455; 435/29

(57) ABSTRACT

Methods for promoting dopaminergic neuronal development and producing neural cells having a dopaminergic phenotype. Dopaminergic neural cells may be used for treating individuals having a neurodegenerative disease such as Parkinson's disease. Dopaminergic cells may be implanted into the brain of the individual, and/or dopaminergic neural development may be induced or enhanced in the brain of the individual. Methods comprise expressing a nuclear receptor of the NG4A subfamily, e.g. Nurr1, above basal levels within the cell and treating the cell with a Wnt ligand, thereby producing or enhancing proliferation, self-renewal, survival and/or dopaminergic induction, differentiation, survival or acquisition of a neuronal dopaminergic phenotype. The cell may be co-cultured with astrocytes or glial cells and may be contacted with an FGF growth factor.

This is a continuation application of U.S. patent application Ser. No. 10/529,097, which is a National Phase application under 35 U.S.C. §371 of International Application No. PCT/IB03/04598, filed Sep. 24, 2003 which, in turn, claims priority benefit from provisional patent application U.S. Ser. No. 60/413,046, filed Sep. 24, 2002 and from provisional patent application U.S. Ser. No. 60/494,595, filed Aug. 12, 2003, each of which is hereby incorporated by reference herein in its entirety.

The present invention relates to induction of neuronal fate in neural stem cells or neural progenitor or precursor cells, or other stem cells. It relates to induction and enhancement of induction of a specific neuronal phenotype, and particularly to induction and enhancement of induction of a midbrain dopaminergic neuronal phenotype.

Parkinson's disease (PD) is a very common neurodegenerative disorder whose pathogenesis is characterized by a selective and progressive loss of midbrain dopaminergic (DA) neurons. The enhancement of induction of neuronal phenotype has the potential to allow for treatment of Parkinson's disease and other seriously debilitating neurodegenerative disorders.

Previously, human fetal mesencephalic tissue has been grafted into Parkinsonian patients with positive results, but development of specific cell replacement therapies utilizing the present invention overcomes practical and ethical difficulties with such prior approaches. In particular, the present invention allows for development of cell preparations for transplantation while reducing or eliminating any need for use of embryo tissue or embyronic cells. Stem cells may be obtained from the umbilical cord, a tissue that is normally discarded. Another option to is to obtain adult stem cells, e.g. from bone marrow, blood, skin, eye, olfactory bulb or olfactory epithelia.

Previously (WO00/66713 and Wagner et al., 1999), the present inventors' laboratory showed that induction of dopaminergic neuronal phenotype is enhanced in cells expressing Nurr1 in the presence of one or more factors obtainable from a Type 1 astrocyte/early glial cell of the ventral mesencephalon. The present invention is based on experimental finding that Wnt factors are useful in enhancing induction of neuronal phenotype of cells expressing Nurr1.

In particular, the inventors have found that all Wnts that are expressed in the VM at higher levels than in the dorsal midbrain by the time of birth of DA neurons are useful in inducing or promoting dopaminergic neuronal development by enhancing proliferation, self-renewal, dopaminergic induction, survival, differentiation and/or maturation in neural stem, progenitor or precursor cells, or other stem or neural cells.

We have found that:

Wnt-1 promotes the proliferation of dopaminergic precursors and the maturation of dopaminergic precursor and/or stem cells into dopaminergic neurons;

Wnt-7a promotes proliferation of dopaminergic precursors and allows their differentiation into dopaminergic neurons;

Wnt-3a promotes proliferation and/or self-renewal of dopaminergic precursor and/or stem cells;

Wnt-2 promotes cell cycle exit and the acquisition of a dopaminergic neuronal phenotype by Nurr1+ precursors; and that

Wnt-5a is the most efficient at inducing a dopaminergic phenotype in neural stem, precursor or progenitor cells, and in enhancing dopaminergic induction or differentiation in a neuronal cell.

Wnt-1 is more efficient than Wnt-3a and Wnt-5a at promoting the proliferation and maturation of dopaminergic precursor and/or stem cells.

The induction of specific neuronal phenotypes requires the integration of both genetic and epigenetic signals. In the developing midbrain, the induction of dopaminergic neurons requires the orphan nuclear receptor Nurr1 (Zetterström et al., 1997; Saucedo-Cárdenas et al., 1998; Castillo et al., 1998), but expression of Nurr1 is not sufficient to induce a dopaminergic phenotype in neural stem cells (Wagner et al., 1999). We previously reported that a combination of Nurr1 and an unknown soluble signal derived from developing ventral midbrain type 1 astrocytes/early glial cells is sufficient to induce a midbrain dopaminergic phenotype in neural stem cells (Wagner et al., 1999). Here we describe that Wnt-5a is part of such signal and that members of the Wnt family of proteins, including Wnt-1, -2, -3a, -5a and -7a are developmentally regulated and differentially control the development of midbrain dopaminergic neurons. Partially purified Wnt-1, -2, -5a, and -7a, but not Wnt-3a, increased the number of E14.5 midbrain DA neurons by two different mechanisms. Wnt-1 and -7a predominantly increased the proliferation of Nurr1 precursors and allowed their differentiation into dopaminergic neurons. Wnt-2 favored cell cycle exit and the acquisition of a dopaminergic neuronal phenotype by Nurr1+ precursors. Wnt-5a mainly increased the proportion of Nurr1 precursors that acquired a neuronal DA phenotype. In agreement with our findings, Wnt-5a was as efficient as midbrain astrocytes/early glial cells at inducing dopaminergic neurons in Nurr1-expressing midbrain or cortical E13.5 precursors. Moreover, the cysteine rich domain of Frizzled 8 efficiently blocked the basal and the VM T1A-, Wnt-1 or Wnt-5a-mediated effects on the increase of cells with a dopaminergic phenotype in Nurr1-expressing neural precursor cultures, and the effect of endogenous Wnts on neural stem cells or FGF-8 expanded Nurr1+ midbrain neurospheres. Thus, the data included herein provide indication that Wnts independently regulate, by partially different mechanisms, the generation of neurons with a DA phenotype in Nurr1-expressing precursors/stem cells.

These findings place Wnt ligands as key regulators of proliferation, self-renewal, differentiation and fate decisions during ventral midbrain neurogenesis. Moreover, our results pave the way for the large scale production of midbrain DA neurons in vitro and for the future implementation of stem cell replacement strategies in the treatment of neurodegenerative diseases such as Parkinson's disease (Bjorklund and Lindvall 2000; Price and Williams 2001; Arenas, 2002 Rossi and Cattaneo, 2002; Gottlieb et al., 2002).

Embryonic, neural and multipotent stem cells have the ability to differentiate into neural cell lineages including neurons, astrocytes and oligodendrocytes. Moreover, stem cells can be isolated, expanded, and used as source material for brain transplants (Snyder, E. Y. et al. Cell 68, 33 51 (1992); Rosenthal, A. Neuron 20, 169 172 (1998); Bain et al., 1995; Gage, F. H., et al. Ann. Rev. Neurosci. 18, 159 192 (1995); Okabe et al., 1996; Weiss, S. et al. Trends Neurosci. 19, 387 393 (1996); Snyder, E. Y. et al. Clin. Neurosci. 3, 310 316 (1996); Martínez Serrano, A. et al. Trends Neurosci. 20, 530 538 (1997); McKay, R. Science 276, 66 71 (1997); Deacon et al., 1998; Studer, L. et al. Nature Neurosci. 1, 290 295 (1998); Bjorklund and Lindvall 2000; Brustle et al., 1999; Lee et al., 2000; Shuldiner et al., 2000 and 2001; Reubinoff et al., 2000 and 2001; Tropepe et al., 2001; Zhang et al., 2001; Price and Williams 2001; Arenas 2002; Bjorklund et al., 2002; Rossi and Cattaneo, 2002; Gottlieb et al., 2002).

Most neurodegenerative diseases affect neuronal populations. Moreover, most of the damage occurs to a specific neurochemical phenotype. In human Parkinson's disease, for example, the major cell type lost is midbrain dopaminergic neurons. Functional replacement of specific neuronal populations through transplantation of neural tissue represents an attractive therapeutic strategy for treating neurodegenerative diseases (Rosenthal, A. Neuron 20, 169 172 (1998)). Another alternative would be the direct infusion of signals required to promote regeneration, repair or guide the development and/or recruitment of stem or progenitor or precursor cells, or the administration of drugs that regulate those functions.

Stem/progenitor or precursor cells are an ideal material for transplantation therapy since they can be expanded and instructed to assume a specific neuronal phenotype. These cells would circumvent ethical and practical issues surrounding the use of human fetal tissue for transplantation. In particular, implanted non-autologous tissue has a limited viability and may be rejected by the immune system. In addition, each fetus provides only a small number of cells.

Induction of a single and specific neuronal phenotype in stem or progenitor or precursor cells has proven elusive.

The present invention provides for induction of dopaminergic neuronal phenotype in cells.

The present invention allows for the induction of dopaminergic neuron development. Thus, by increasing Wnt levels and/or function in cultures or in the brain, the invention allows the induction or promotion of: proliferation and/or self-renewal of dopaminergic precursors, progenitor or stem cells; and/or promotion of dopaminergic neuron, precursor, progenitor or stem cell survival, differentiation and maturation, increasing the yield of dopaminergic neurons; and/or induction of a neuronal dopaminergic fate in stem, progenitor, precursor or neuronal cells in vitro or in vivo.

Any aspect or embodiment of the invention can apply to or use a neuronal cell i.e. a neuron. A ‘neural cell’ in the present disclosure may be a neuronal cell.

Cell preparations rich in dopaminergic neurons may be used for cell replacement therapy in Parkinson's disease or other disorders, and for studying signaling events in dopaminergic neurons and the effects of drugs on dopaminergic neurons in vitro, for instance in high throughput screening.

Aspects and embodiments of the present invention are provided as set out in the claims below.

In one aspect, the present invention provides a method of inducing a dopaminergic neuronal fate in a stem cell, neural stem cell or neural progenitor or precursor cell, or enhancing dopaminergic induction or differentiation in a neuronal cell, or expanding a dopaminergic precursor or progenitor or a Nurr1-expressing stem cell, the method comprising:

expressing a nuclear receptor of the Nurr1 subfamily above basal levels within the cell, and

treating the cell with a Wnt ligand,

whereby dopaminergic neurons are produced.

The Nurr1 subfamily, also known as the NR4A subfamily, includes Nurr1/NR4A2, Nor1/NR4A3 and NGFI-B/NR4A1. Accordingly, methods of the invention may comprise expressing, for example, Nurr1/NR4A2, Nor1/NR4A3 and/or NGFI-B/NR4A1 above basal levels within the cell. Preferably the nuclear receptor of the Nurr1 subfamily is Nurr1. Thus, methods of the invention preferably comprise expressing Nurr1 above basal levels within the cell.

The invention provides a method of inducing or promoting dopaminergic neuronal development by enhancing proliferation, self-renewal, dopaminergic induction, survival, differentiation and/or maturation in a neural stem, progenitor or precursor cell, or other stem or neural cell, the method comprising:

expressing Nurr1 above basal levels within the cell, and

treating the cell with a Wnt ligand,

thereby producing or enhancing proliferation, self-renewal, survival and/or dopaminergic induction, differentiation, survival or acquisition of a neuronal dopaminergic phenotype.

Treating with a Wnt ligand may be in vivo, ex vivo, or in culture.

In methods of the invention, treating with a Wnt ligand may be by means of contacting a cell with the ligand. Treating with a Wnt ligand may be by means of provision of purified and/or recombinant Wnt ligand to a culture comprising the stem, progenitor or precursor cell, or to such a cell in vivo. Treating with a Wnt ligand may comprise introducing one or more copies of Wnt nucleic acid or protein into the cell. Methods of transforming cells with nucleic acid and introducing proteins into cells are described further below. Contacting with a Wnt ligand may be by means of providing in vivo or within a culture comprising the stem, progenitor or precursor cell or neuronal cell a cell that produces the Wnt ligand. The cell that produces the Wnt ligand may be a recombinant host cell that produces Wnt ligand by recombinant expression. A co-cultured host cell may be transformed with nucleic acid encoding a Wnt ligand, and/or the co-cultured cell may contain introduced Wnt protein. Wnt protein, or nucleic acid encoding Wnt, may be introduced into the cell in accordance with available techniques in the art, examples of which are described below.

The co-cultured or host cell may be another stem, neural stem, progenitor, precursor or neural cell. Treatment with a Wnt ligand may also be by means of upregulating Wnt expression in the cell or by downregulating or inhibiting an inhibitor molecule of the Wnt ligand. Thus treatment with a Wnt ligand may arise by decreasing expression or activity of Wnt-interacting molecules, such as SFRP, WIF, dkk or Cerberus (Martinez Arias et al., 1999; http://www.stanford.edu/˜rnusse/wntwindow.html, or findable using any web browser).

In addition to provision of Wnt ligand, the stem cell or neural stem, progenitor or precursor cell or neuronal cell may be in co-culture with Type 1 astrocytes/glial cells, or in contact with such cells or factors derived from them in vitro or in vivo. However, in accordance with the present invention reliance is not placed on the Type 1 astrocytes to provide Wnt ligand, and a co-culture containing a neural stem cell or neural progenitor cell and Type 1 astrocytes without extraneous provision of Wnt ligand is not contemplated within the scope of the present invention, nor are methods employing such a co-culture.

Nurr1 (Law, et al., 1992; Xing, et al., 1997; Castillo, 1997; GenBank nos. S53744, U72345, U86783) is a transcription factor of the thyroid hormone/retinoic acid nuclear receptor superfamily. As shown previously in WO00/66713 and Wagner et al., 1999, expression of Nurr1 above basal levels in neural stem cells or neural progenitor cells increases the proportion of the cells which differentiate toward a neuronal fate. The induction of a neuronal fate may be carried out in vitro or in vivo. The ability to induce differentiation of stem cells or neural stem, progenitor or precursor cells toward the neuronal fate prior to, or following transplantation, ameliorates the biasing of transplanted stem cells to differentiate into astroglial fates when grafted in the adult brain. Nurr1 is a member of the NR4A subfamily. Methods of the invention are not limited to Nurr1, although Nurr1 may be preferred, and methods may comprise expressing any nuclear receptor of the NR4A subfamily above basal levels in the cell. Receptors of the NR4A subfamily include Nurr1/NR4A2, Nor1/NR4A3 and NGFI-B/NR4A1. Thus, in methods of the invention, a nuclear receptor of the NR4A subfamily (e.g. Nurr1, Nor1 or NGFI-B) may be expressed above basal levels within the cell. Accession numbers for example NR4A subfamily members are as follows:

NGF-IB protein: NP775181 NP775180 NP002126
NGFI-B nucleotide: NM_{—}173158 NM_{—}173157 NM_{—}002135
Nor-1 protein: NP775292 NP775291 NP775290 NP008912 S71930 Q92570
Nor-1 nucleotide: NM_{—}005413 NM_{—}173200 NM_{—}173199 NM_{—}173198 NM_{—}006981

Members of the Wnt family of glycoproteins are poorly soluble (Bradley and Brown, 1990 and 1995) and expressed in the developing mesencephalon (Parr et al., 1993). Wnts regulate midbrain-hindbrain development (McMahon and Bradley, 1990; Thomas and Capecchi, 1990), neural patterning (Kiecker and Niehrs, 2001; Nordström et al., 2002; Houart et al., 2002), precursor proliferation (Taipale and Beachy, 2001; Chenn and Walsh, 2002; Megason and McMahon, 2002) and fate decisions in multiple tissues (Kispert et al., 1998; Ross et al., 2000; Hartmann and Tabin, 2001; Marvin et al., 2001; Schneider and Mercola, 2001; Tzahor and Lassar, 2001; Pandur et al., 2002), including the nervous system (Dorsky et al., 1998; Baker et al., 1999; Wilson et al., 2001; Garcia-Castro et al., 2002; Muroyama et al., 2002).

As used herein, a “Wnt polypeptide”, “Wnt glycoprotein” or “Wnt ligand” refers to a member of the Wingless-int family of secreted proteins that regulate cell-to-cell interactions. Wnts are highly conserved from Drosophila and Caenorhabditis elegans, to Xenopus, zebra fish and mammals. The 19 Wnt proteins currently known in mammals bind to two cell surface receptor types: the seven transmembrane domain Frizzled receptor family, currently formed by 10 receptors, and the Low density lipoprotein-receptor related proteins (LRP) 5 and 6 and the kremen 1 and 2 receptors. The signal conveyed by Wnts is transduced via three known signaling pathways: (1) the so called canonical signaling pathway, in which GSK3 beta is inhibited, does not phosphorylate beta-catenin, which is then not degraded and is translocated to the nucleus to form a complex with TCF and activate transcription of Wnt target genes. (2) the planar polarity and convergence-extension pathway, via Jnk. (3) and the inositol 1,4,5 triphosphate (IP3)/calcium pathway, in which calcineurin dephosphorylates and activates the nuclear factor of activated T cells (NF-AT) (Saneyoshi et al., 2002). For review see the Wnt home page, findable on the web using any available browser (www.stanford.edu/˜rnusse/wntwindow.html. Other co-receptors involved in Wnt signaling include the tyrosine kinase receptor Ror1 and Ror 2 (Oishi I et al., 2003), the derailed/RYK receptor family (Yoshikawa et al., 2003), which encode catalytically inactive receptor tyrosine kinases.

In some preferred embodiments of the various aspects of the present invention the Wnt ligand is a Wnt1 ligand. Human Wnt1 amino acid sequence is available under GenBank reference Swiss protein accession number P04628 and encoding nucleic acid under reference X03072.1 for DNA and NM_{—}005430.2 for RNA. Human Wnt1 nucleic acid can be amplified using primers with SEQ ID NO:'s 1 and 2, identified further below.

In some preferred embodiments of the various aspects of the present invention the Wnt ligand is a Wnt5a ligand. Human Wnt5a amino acid sequence is available under GenBank reference Swiss protein accession number P41221. and encoding nucleic acid under reference AI634753.1 AK021503 L20861 L20861.1 U39837.1 for DNA and NM_{—}003392 for RNA. Human Wnt5a nucleic acid can be amplified using primers with SEQ ID NOS: 5 and 6, identified further below.

Although not preferred for generation of dopaminergic neurons, some preferred embodiments of the present invention may employ a Wnt3a ligand. Various aspects and embodiments of the invention analogous to those disclosed herein for generation and use of dopaminergic neurons are provided by the present invention in which a Wnt3a ligand is used to maintain the proliferation or self-renewal of stem/progenitor cells and/or allow or induce their differentiation into other, i.e. non-dopaminergic, neuronal phenotypes. As demonstrated by the experiments described herein, Wnt3a decreases the number of Nurr-1 expressing progenitors that give rise to dopaminergic neurons. However, since the total number of neurons is not decreased other neuronal phenotypes may be produced, e.g. dorsal midbrain phenotypes, including serotonergic neurons. Loss of serotonergic neurons is associated with depression, so neurons generated by methods comprising use of a Wnt3a ligand, and/or a Wnt3a ligand itself, may be used in therapies e.g. of depression.

Human Wnt3a amino acid sequence is available under GenBank reference SwissProt Accession No. P56704 and encoding nucleic acid under reference AB060284 AB060284.1 AK056278 AK056278.1 for DNA and NM_{—}033131 for mRNA. Human Wnt3a nucleic acid can be amplified using primers with SEQ ID NO:'s 3 and 4, identified further below.

Other preferred Wnts are Wnt-2, Wnt-4, Wnt-7a and Wnt-7b, especially Wnt-2 and Wnt-7a. Wnt-2 may be used to promote and/or induce acquisition of neuronal phenotype, and differentiation and/or maturation of stem cells and of neural stem, precursor and progenitor cells into DA neurons. Wnt-7a may be used to promote and/or induce proliferation of stem cells and neural stem, precursor and progenitor cells, and thereby to promote differentiation and/or maturation of the cells into DA neurons. Either or both of Wnt-2 and Wnt-7a may be used to increase the number of TH+ neurons from Nurr1+ cells.

Wnt-2 nucleic acid is deposited under GenBank reference SwissProt Accession No. P09544, NCBI RefSeq protein NP00382, NCBI RefSeq mRNA NM_{—}003391 and NM_{—}003391.1, NCBI RefSeq DNA NT_{—}007933 and nucleic acid under references AK056742 AK056742.1 BC029854 BC029854.1 X07876 X07876.1 AC002465 AC006326 and can be amplified using primers with SEQ ID NOS: 33 and 34, as described below. Wnt-4 nucleic acid is deposited under GenBank reference SwissProt Accession No. P56705, NCBI RefSeq protein NP110388, NCBI RefSeq mRNA NM_{—}030761 and NM_{—}030761.2, NCBI RefSeq DNA NT_{—}004610 and nucleic acid under references AA984007 AL031281 AY009398.1 AF009398.1 AB062766.1 BC034923.1 AF416743.1 AB061675.1 BQ891671.1 BU502468.1 BM043406.1 CB991983.1 and can be amplified using primers with SEQ ID NOS: 39 and 40, as described below. Wnt-7a nucleic acid is deposited under GenBank reference SwissProt Accession No. 000755, NCBI RefSeq protein NP004616 and NP004616.2, NCBI RefSeq mRNA NM_{—}004625 and NM_{—}004625.2, NCBI RefSeq DNA NT_{—}005927 and nucleic acid under references D83175 D83175.1 BC008811 BC008811.1 U53476 U53476.1 BI823772.1 CB989433.1 BI552826.1 BI551057.1 BE740508.1 and can be amplified using primers with SEQ ID NOS: 45 and 46, as described below. Wnt-7b nucleic acid is deposited under GenBank reference SwissProt Accession No. P56706, NCBI RefSeq protein NP478679 and NP478679.1, NCBI RefSeq mRNA NM_{—}004625 and NM_{—}004625.2 and nucleic acid under references AA062766 AF416743 BC034923 BM047487 BM047487.1 BU 543397.1 BU541891.1 BU541105.1 and can be amplified using primers with SEQ ID NOS: 47 and 48, as described below.

A wild-type Wnt ligand may be employed, or a variant or derivative, e.g. by addition, deletion, substitution and/or insertion of one or more amino acids, provided the function of enhancing development of a dopaminergic neuronal fate in a stem cell, neural stem cell or neural progenitor or precursor cell is retained.

By “stem cell” is meant any cell type that can self renew and, if it is an embryonic stem (ES) cell, can give rise to all cells in an individual, or, if it is a multipotent or neural stem cell, can give rise to all cell types in the nervous system, including neurons, astrocytes and oligodendrocytes. A stem cell may express one or more of the following markers: Oct-4; Sox1-3; stage specific embryonic antigens (SSEA-1, -3, and -4), and the tumor rejection antigens TRA-1-60 and -1-81, as described (Tropepe et al., 2001; Xu et al., 2001). A neural stem cell may express one or more of the following markers: Nestin; the p75 neurotrophin receptor; Notch1, SSEA-1 (Capela and Temple, 2002).

By “neural progenitor cell” is meant a daughter or descendant of a neural stem cell, with a more differentiated phenotype and/or a more reduced differentiation potential compared to the stem cell. By precursor cell it is meant any other cell being or not in a direct lineage relation with neurons during development but that under defined environmental conditions can be induced to transdifferentiate or redifferentiate or acquire a neuronal phenotype. In preferred embodiments, the stem, neural stem, progenitor, precursor or neural cell does not express or express efficiently tyrosine hydroxylase either spontaneously or upon deprivation of mitogens (e.g. bFGF, EGF or serum).

A stem cell, neural stem cell or neural progenitor or precursor cell may be obtained or derived from any embryonic, fetal or adult tissue, including bone marrow, skin, eye, nasal epithelia, or umbilical cord, or region of the nervous system, e.g. from the cerebellum, the ventricular zone, the sub-ventricular zone, the striatum, the midbrain, the hindbrain, the cerebral cortex or the hippocampus. It may be obtained or derived from a vertebrate organism, e.g. from a mammal, which may be human or non-human, such as rabbit, guinea pig, rat, mouse or other rodent, cat, dog, pig, sheep, goat, cattle, horse, or primate, or from a bird, such as a chicken.

In preferred embodiments of the present invention, adult stem/progenitor/precursor cells are used, in vitro, ex vivo or in vivo. This requires a consenting adult (e.g. from which the cells are obtained) and approval by the appropriate ethical committee. If a human embryo/fetus is used as a source, the human embryo is one that would otherwise be destroyed without use, or stored indefinitely, especially a human embryo created for the purpose of IVF treatment for a couple having difficulty conceiving. IVF generally involves creation of human embryos in a number greater than the number used for implantation and ultimately pregnancy. Such spare embryos may commonly be destroyed. With appropriate consent from the people concerned, in particular the relevant egg donor and/or sperm donor, an embryo that would otherwise be destroyed can be used in an ethically positive way to the benefit of sufferers of severe neurodegenerative disorders such as Parkinson's disease. The present invention itself does not concern the use of a human embryo in any stage of its development. As noted, the present invention minimizes the possible need to employ a material derived directly from a human embryo, whilst allowing for development of valuable therapies for terrible diseases.

In some preferred embodiments, a stem or progenitor or precursor cell contacted with a Wnt ligand and otherwise treated and/or used in accordance with any aspect of the present invention is obtained from a consenting adult or child for which appropriate consent is given, e.g. a patient with a disorder that is subsequently treated by transplantation back into the patient of neurons generated in accordance with the invention, and/or treated with one or more Wnt ligands and/or one or more type 1 astrocyte/early glial cell-derived factors to promote or induce endogenous dopaminergic neuron development or function.

The neuronal fate to which the stem or progenitor or precursor cell is induced may exhibit an undifferentiated phenotype or a primitive neuronal phenotype. It may be a totipotent cell, capable of giving rise to any cell type in an individual, or a multipotent cell which is capable of giving rise to a plurality of distinct neuronal phenotypes, or a precursor or progenitor cell, capable of giving rise to more limited phenotype during normal development but capable of giving rise to other cells when exposed to appropriate environmental factors in vitro. It may lack markers associated with specific neuronal fates, e.g. tyrosine hydroxylase.

In a method of inducing a neuronal fate according to the present invention wherein a plurality of stem cells, neural stem cells and/or progenitor cells and/or precursor cells express Nurr1, or another nuclear receptor of the Nurr1 subfamily, above basal levels, and the cells are treated with Wnt ligand, a majority of the cells may be induced to adopt a neuronal fate. Dopaminergic induction or differentiation may be enhanced in neuronal cells. In preferred embodiments, more than 60%, more than 70%, more than 80%, more than 90% of the stem and/or progenitor cells may be induced to a neuronal fate.

By “expressing Nurr1 above basal levels within the cell” is meant expressing Nurr1 at levels greater than that at which it is expressed in the (unmodified) cell in vivo under non-pathological conditions. Likewise, expressing a nuclear receptor of the Nurr1 subfamily above basal levels within the cell means expressing the nuclear receptor at levels greater than that at which it is expressed in the (unmodified) cell in vivo under non-pathological conditions. Expression above basal levels includes transcriptional, translational, posttranslational, pharmacological, artificial upregulation and over-expression. Expression of nuclear receptors above basal levels is described herein with reference to Nurr1. This disclosure is also applicable to other members of the Nurr1 subfamily and may be used in methods of the invention with other nuclear receptors of that subfamily e.g. Nor1 or NGFI-B. Thus, although Nurr1 is exemplified, methods of the invention are not limited to Nurr1 and extend to any nuclear receptor of the Nurr1 subfamily.

Expression of Nurr1 above basal levels may be achieved by any method known to those skilled in the art. By way of example, expression above basal levels may be induced by modulating the regulation of native genomic Nurr1. This may be done by inhibiting or preventing degradation of Nurr1 mRNA or protein or by increasing transcription and/or translation of Nurr1, e.g. by contacting the cell with fibroblast growth factor 8 (FGF8), which upregulates transcription of Nurr1 (Rosenthal, A., (1998) Cell, 93(5), 755-766), and/or by introducing heterologous regulatory sequences into or adjacent the native regulatory region of Nurr1, and/or by replacing the native regulatory region of Nurr1 with such heterologous regulatory sequences, e.g. by homologous recombination, and/or by disrupting or downregulating molecules that negatively regulate, block or downregulate transcription, translation or the function of Nurr1, e.g. Nurr2 (Ohkura, et al., (1999) Biochim Biophys Acta 14444: 69-79).

Transcription may be increased by providing the stem, neural stem, precursor, progenitor or neural cell with increased levels of a transcriptional activator, e.g. by contacting the cell with such an activator or by transformation of the cell with nucleic acid encoding the activator. Alternatively, transcription may be increased by transforming the cell with antisense nucleic acid to a transcriptional inhibitor of Nurr1.

Accordingly, a method of the present invention of inducing or enhancing induction of a neuronal fate in a stem, neural stem, precursor, progenitor cell, or neural cell, may include contacting the cell with FGF8 or FGF20 (Ohmachi et al., 2000).

As an alternative or addition to increasing transcription and/or translation of endogenous Nurr1, expression of Nurr1 above basal levels may be caused by introduction of one or more extra copies of Nurr1 into the stem, neural stem, precursor, progenitor or neural cell.

Accordingly, in a further aspect, the present invention provides a method of inducing a neuronal fate and/or enhancing the induction of dopaminergic development in a stem cell, neural stem cell, neural progenitor, precursor or neural cell, or enhancing dopaminergic induction or differentiation in a neuronal cell, the method including, in addition to contacting the cell with Wnt ligand, transforming the cell with Nurr1.

Transformation of the stem, neural stem, precursor or progenitor cell or neuronal cell may be carried out in vitro, in vivo or ex vivo. The neuronal fate to which the cell is induced may be of the type discussed herein, e.g. it may exhibit a primitive neuronal phenotype and may lack markers associated with specific neuronal fates. The invention further provides a stem cell, neural stem cell or neural progenitor or precursor cell transformed with Nurr1 and contacted with Wnt ligand.

Transformed Nurr1 and/or Wnt ligand may be contained on an extra-genomic vector or it may be incorporated, preferably stably, into the genome. It may be operably-linked to a promotor which drives its expression above basal levels in stem cells, or neural stem, precursor or progenitor cells, or neuronal cells, as is discussed in more detail below.

“Operably linked” means joined as part of the same nucleic acid molecule, suitably positioned and oriented for transcription to be initiated from the promoter.

Methods of introducing genes into cells are well known to those skilled in the art. Vectors may be used to introduce Nurr1 and/or Wnt ligand into stem, or neural stem, precursor or progenitor cells or neuronal cells, whether or not the Nurr1 and/or Wnt ligand remains on the vector or is incorporated into the genome. Suitable vectors can be chosen or constructed, containing appropriate regulatory sequences, including promoter sequences, terminator fragments, polyadenylation sequences, enhancer sequences. Vectors may contain marker genes and other sequences as appropriate. The regulatory sequences may drive expression of Nurr1 and/or Wnt ligand within the stem, or neural stem, precursor or progenitor cells or neural cells. For example, the vector may be an extra-genomic expression vector, or the regulatory sequences may be incorporated into the genome with Nurr1 and/or Wnt ligand. Vectors may be plasmids or viral.

Nurr1 and/or Wnt ligand may be placed under the control of an externally inducible gene promoter to place it under the control of the user. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Whatever the level of expression is in the absence of the stimulus, expression from any inducible promoter is increased in the presence of the correct stimulus. An example of an inducible promoter is the Tetracyclin ON/OFF system (Gossen, et al., 1995) in which gene expression is regulated by tetracyclin analogs.

For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992 or later edition.

Marker genes such as antibiotic resistance or sensitivity genes may be used in identifying clones containing nucleic acid of interest, as is well known in the art. Clones may also be identified or further investigated by binding studies, e.g. by Southern blot hybridisation.

Nucleic acid including Nurr1 and/or encoding Wnt ligand may be integrated into the genome of the host stem, neural stem, progenitor, precursor or neural cell. Integration may be promoted by including in the transformed nucleic acid sequences which promote recombination with the genome, in accordance with standard techniques. The integrated nucleic acid may include regulatory sequences able to drive expression of the Nurr1 gene and/or Wnt ligand in a stem cell, or neural stem, progenitor or precursor cells, or neuronal cells. The nucleic acid may include sequences which direct its integration to a site in the genome where the Nurr1 and/or Wnt ligand coding sequence will fall under the control of regulatory elements able to drive and/or control its expression within the stem, or neural stem, precursor or progenitor cell, or neuronal cell. The integrated nucleic acid may be derived from a vector used to transform Nurr1 and/or Wnt ligand into the stem cell, or neural stem, precursor or progenitor cells, or neuronal cells, as discussed herein.

The introduction of nucleic acid comprising Nurr1 and/or encoding Wnt ligand, whether that nucleic acid is linear, branched or circular, may be generally referred to without limitation as “transformation”. It may employ any available technique. Suitable techniques may include calcium phosphate transfection, DEAE-Dextran, PEI, electroporation, mechanical techniques such as microinjection, direct DNA uptake, receptor mediated DNA transfer, transduction using retrovirus or other virus and liposome- or lipid-mediated transfection. When introducing a chosen gene construct into a cell, certain considerations must be taken into account, well known to those skilled in the art. It will be apparent to the skilled person that the particular choice of method of transformation to introduce Nurr1 and/or Wnt ligand into a stem cell, or neural stem, precursor or progenitor cells or a neuronal cell is not essential to or a limitation of the invention.

Suitable vectors and techniques for in vivo transformation of stem cells, or neural stem, precursor or progenitor cells or neuronal cells with Nurr1 and/or Wnt ligand are well known to those skilled in the art. Suitable vectors include adenovirus, adeno-associated virus papovavirus, vaccinia virus, herpes virus, lentiviruses and retroviruses. Disabled virus vectors may be produced in helper cell lines in which genes required for production of infectious viral particles are expressed. Suitable helper cell lines are well known to those skilled in the art. By way of example, see: Fallaux, F. J., et al., (1996) Hum Gene Ther 7(2), 215-222; Willenbrink, W., et al., (1994) J Virol 68(12), 8413-8417; Cosset, F. L., et al., (1993) Virology 193(1), 385-395; Highkin, M. K., et al., (1991) Poult Sci 70(4), 970-981; Dougherty, J. P., et al., (1989) J Virol 63(7), 3209-3212; Salmons, B., et al., (1989) Biochem Biophys Res Commun 159(3), 1191-1198; Sorge, J., et al., (1984) Mol Cell Biol 4(9), 1730-1737; Wang, S., et al., (1997) Gene Ther 4(11), 1132-1141; Moore, K. W., et al., (1990) Science 248(4960), 1230-1234; Reiss, C. S., et al., (1987) J Immunol 139(3), 711-714. Helper cell lines are generally missing a sequence which is recognised by the mechanism which packages the viral genome. They produce virions which contain no nucleic acid. A viral vector which contains an intact packaging signal along with the gene or other sequence to be delivered (e.g. Nurr1 and/or Wnts) is packaged in the helper cells into infectious virion particles, which may then be used for gene delivery to stem cells, or neural stem, precursor or progenitor cells or neuronal cells.

As an alternative or addition to increasing transcription and/or translation of endogenous Nurr1 and/or Wnts, expression of Nurr1 and/or Wnts above basal levels may be caused by introduction of one or more extra copies of Nurr1 and/or Wnts protein into the stem, neural stem, precursor, progenitor or neural cell by microinjection or other carrier-based or protein delivery system including cell penetrating peptides, i.e.: TAT, transportan, Antennapedia penetratin peptides (Lindsay 2002).

In a further aspect, the present invention provides a method of inducing a specific neuronal fate in a stem, neural stem or progenitor or precursor cell, or neuronal cell, wherein the stem cell or progenitor cell or neuronal cell expresses Nurr1 above basal levels, the method including contacting the cells with a Wnt ligand and optionally one or more factors supplied by or derived from a Type 1 astrocyte/glial cell. The factor or factors may be provided by co-culturing or contacting the stem, progenitor or precursor cell or neuronal cell with a Type 1 astrocyte/glial cell. The method may occur in vitro or in vivo. The stem cell or neural stem, precursor or progenitor cells or neuronal cells expressing Nurr1 and/or Wnts above basal levels may be produced by transformation of the cells with Nurr1 and/or Wnts.

The factor or factors may be supplied by or derived from an immortalized astrocyte/glial cell. The factor or factors may be supplied by or derived from a glial cell line, e.g. an astrocyte or radial glia or immature glial mesencephalic cell line. Cell lines provide a homogenous cell population.

Important aspects of the present invention are based on the finding that, whereas dopaminergic neurons can be generated from stem cells or progenitor or precursor cells in vitro by a process including expression of Nurr1 above basal levels in the cells and contact of the cells with one or more factors supplied by or derived from Type 1 astrocytes/early glial cells of the ventral mesencephalon, induction of dopaminergic fate is enhanced or promoted by contact with a Wnt ligand.

The present invention allows for generation of large numbers of dopaminergic neurons. These dopaminergic neurons may be used as source material to replace cells which degenerate or are damaged or lost in Parkinson's disease.

Preferably, the cell expressing Nurr1 above basal levels is mitotic when it is contacted with Wnt ligand.

In methods of the invention, the cell may additionally be contacted with one or more agents selected from: basic fibroblast growth factor (bFGF); epidermal growth factor (EGF); and an activator of the retinoid X receptor (RXR), e.g. the synthetic retinoid analog SR11237, (Gendimenico, G. J., et al., (1994) J Invest Dermatol 102(5), 676-80), 9-cis retinol or docosahexanoic acid (DHA) or LG849 (Mata de Urquiza et al., 2000). Treating cells in accordance with the invention with one or more of these agents may be used to increase the proportion of the stem, progenitor or precursor cells which adopt a dopaminergic fate, or enhance dopaminergic induction or differentiation in a neuronal cell, as demonstrated experimentally below. The method of inducing a dopaminergic fate or enhancing dopaminergic induction or differentiation in a neuronal cell in accordance with the present invention may include contacting the cell with a member of the FGF family of growth factors, e.g. FGF4, FGF8 or FGF20.

Advantageously, the cells may be contacted with two or more of the above agents. The inventors have unexpectedly found that the beneficial effects of bFGF or EGF and SR11237 are additive at saturating doses. This finding suggests that these agents may act through different mechanisms.

The method of inducing a dopaminergic phenotype may include pretreating the stem cell, neural stem cell or neural progenitor or precursor cell or neuronal cell with bFGF and/or EGF prior to contacting it with Wnt ligand and optionally one or more further factors supplied by or derived from Type 1 astrocytes/glial cells of the ventral mesencephalon, e.g. prior to contacting or co-culturing it with ventral mesencephalic Type 1 astrocytes/glial cells or factors derived from them.

The optional pretreatment step arises from two further unexpected findings of the inventors that were previously reported in WO00/66713 and Wagner et al. (1999): (i) that neural stem cell lines expressing Nurr1 above basal levels and showing high proliferation demonstrate enhanced induction to dopaminergic fate when co-cultured with Type 1 astrocytes/glial cells of the ventral mesencephalon; and (ii) that after treatment with bFGF or EGF in serum-free medium (SFM), the baseline proliferation of most stem cell lines expressing Nurr1 above basal levels remained elevated after passage into SFM alone.

The method of inducing a dopaminergic phenotype may include pretreating a stem cell or neural stem, progenitor or precursor cell with a member of the FGF family of growth factors, e.g. FGF2, FGF4, FGF8 or FGF20.

Instead or as well as pretreating, the additional factors may be to treat cells simultaneously with Wnt treatment.

A method according to the invention in which a neuronal fate is induced in a stem, neural stem or progenitor or precursor cell or there is enhanced dopaminergic induction or differentiation in a neuronal cell, may include detecting a marker for the neuronal fate. b-tubulin III (TuJ1) is one marker of the neuronal fate (Menezes, J. R., et al., (1994) J Neurosci 14(9), 5399-5416). Other neuronal markers include neurofilament and MAP2. If a particular neuronal phenotype is induced, the marker should be specific for that phenotype. For the dopaminergic fate, expression of tyrosine hydroxylase (TH), dopamine transporter (DAT) and dopamine receptors may be detected e.g. by immunoreactivity or in situ hybridization. Tyrosine hydroxylase is a major marker for DA cells. Contents and/or release of dopamine and metabolites may be detected e.g. by High Pressure Liquid Chromatography (HPLC) (Cooper, J. R., et al., The Biochemical Basis of Neuropharmacology, 7th Edition, (1996) Oxford University Press). The absence of Dopamine β hydroxylase and GABA or GAD (in the presence of TH/dopamine/DAT) is also indicative of dopaminergic fate. Additional markers include Aldehyde dehydrogenase type 2 (ADH-2), GIRK2, Lmx1b and Ptx3.

Detection of a marker may be carried out according to any method known to those skilled in the art. The detection method may employ a specific binding member capable of binding to a nucleic acid sequence encoding the marker, the specific binding member comprising a nucleic acid probe hybridisable with the sequence, or an immunoglobulin/antibody domain with specificity for the nucleic acid sequence or the polypeptide encoded by it, the specific binding member being labelled so that binding of the specific binding member to the sequence or polypeptide is detectable. A “specific binding member” has a particular specificity for the marker and in normal conditions binds to the marker in preference to other species. Alternatively, where the marker is a specific mRNA, it may be detected by binding to specific oligonucleotide primers and amplification in e.g. the polymerase chain reaction.

Nucleic acid probes and primers may hybridize with the marker under stringent conditions. Suitable conditions include, e.g. for detection of marker sequences that are about 80-90% identical, hybridization overnight at 42 C in 0.25M Na2HPO4, pH 7.2, 6.5% SDS, 10% dextran sulfate and a final wash at 55 C in 0.1×SSC, 0.1% SDS. For detection of marker sequences that are greater than about 90% identical, suitable conditions include hybridization overnight at 65 C in 0.25M Na2HPO4, pH 7.2, 6.5% SDS, 10% dextran sulfate and a final wash at 60 C in 0.1×SSC, 0.1% SDS.

In a further aspect, the present invention provides a neuron produced in accordance with any one of the methods disclosed herein. The neuron may have a primitive neuronal phenotype. It may be capable of giving rise to a plurality of distinct neuronal phenotypes. The neuron may have a particular neuronal phenotype, the phenotype being influenced by the Wnt ligand and/or the type of astrocytes/glial cells from which the factor or factors which contacted the stem, neural stem, progenitor, precursor or neural cell expressing Nurr1 above basal levels were supplied or derived, and/or by the type of astrocyte/glial cell with which the stem, neural stem, progenitor, precursor or neural cell was co-cultured or contacted. In preferred embodiments, the neuron has a dopaminergic phenotype.

The neuron may contain nucleic acid encoding a molecule with neuroprotective or neuroregenerative properties operably linked to a promoter which is capable of driving expression of the molecule in the neuron. The promoter may be an inducible promoter, e.g. the TetON chimeric promoter, so that any damaging over-expression may be prevented. The promoter may be associated with a specific neuronal phenotype, e.g. the TH promoter or the Nurr1 promoter.

The encoded molecule may be such that its expression renders the neuron independent of its environment, i.e. such that its survival is not dependent on the presence of one or more factors or conditions in e.g. the neural environment into which it is to be implanted. By way of example, the neuron may contain nucleic acid encoding one or more of the neuroprotective or neuroregenerative molecules described below operably linked to a promoter which is capable of driving expression of the molecule in the neuron.

In addition or alternatively, expression of the encoded molecule may function in neuroprotection or neuroregeneration of the cellular environment surrounding that neuron. In this way, the neuron may be used in a combined cell and gene therapy approach to deliver molecules with neuroprotective and neuroregenerative properties.

Examples of molecules with neuroprotective and neuroregenerative properties include:

(i) neurotropic factors able to compensate for and prevent neurodegeneration. One example is glial derived neurotropic growth factor (GDNF) which is a potent neural survival factor, promotes sprouting from dopaminergic neurons and increases tyrosine hydroxylase expression (Tomac, et al., (1995) Nature, 373, 335-339; Arenas, et al., (1995) Neuron, 15, 1465-1473). By enhancing axonal elongation GDNF, GDNF may increase the ability of the neurons to inervate their local environment. Other neurotropic molecules of the GDNF family include Neurturin, Persephin and Artemin. Neurotropic molecules of the neurotropin family include nerve growth factor (NGF), brain derived neurotropic factor (BDNF), and neurotropin-3, -4/5 and -6. Other factors with neurotrophic activity include members of the FGF family for instance FGF2, 4, 8 and 20; members of the Wnt family, including Wnt-1, -2, -5a, -3a and 7a; members of the BMP family, including BMP2, 4, 5 and 7, nodal, activins and GDF; and members of the TGFalpha/beta family.
(ii) antiapoptotic molecules. Bcl2 which plays a central role in cell death. Over-expression of Bcl2 protects neurons from naturally occurring cell death and ischemia (Martinou, et al., (1994) Neuron, 1017-1030). Another antiapoptotic molecule specific for neurons is BclX-L.
(iii) axon regenerating and/or elongating and/or guiding molecules which assist the neuron in innervating and forming connections with its environment, e.g. ephrins. Ephrins define a class of membrane-bound ligands capable of activating tyrosine kinase receptors. Ephrins have been implicated in neural development (Irving, et al., (1996) Dev. Biol., 173, 26-38; Krull, et al., (1997) Curr. Biol. 7, 571-580; Frisen, et al., (1998) Neuron, 20, 235-243; Gao, et al., (1996) PNAS, 93, 11161-11166; Torres, et al., (1998) Neuron, 21, 1453-1463; Winslow, et al., (1995) Neuron, 14, 973-981; Yue, et al., (1999) J Neurosci 19(6), 2090-2101.
(iv) transcription factors, e.g. the homeobox domain protein Ptx3 (Smidt, M. P., et al., (1997) Proc Natl Acad Sci USA, 94(24), 13305-13310), Lmx1b, Pax2, Pax5, Pax8, or engrailed 1 or 2 (Wurst and Bally-Cuif, 2001; Rhinn and Brand, 2001), or neurogenic genes of the basic helix-loop-helix family.

A neuron in accordance with or for use in the present invention may be substantially free from one or more other cell types, e.g. from stem, neural stem, precursor or progenitor cells. Neurons may be separated from neural stem or progenitor cells using any technique known to those skilled in the art, including those based on the recognition of extracellular epitopes by antibodies and magnetic beads or fluorescence activated cell sorting (FACS). By way of example, antibodies against extracellular regions of molecules found on stem, neural stem, precursor or progenitor cells but not on neurons may be employed. Such molecules include Notch 1, CD133, SSEA1, prominin1/2, RPTPβ/phosphocan, TIS21 and the glial cell line derived neurotrophic factor receptors GFR alphas or NCAM. Stem cells bound to antibodies may be lysed by exposure to complement, or separated by, e.g. magnetic sorting (Johansson, et al., (1999) Cell, 96, 25-34). If antibodies which are xenogeneic to the intended recipient of the neurons are used, then any e.g. stem, neural stem or progenitor or precursor cells which escape such a cell sorting procedure are labelled with xenogeneic antibodies and are prime targets for the recipient's immune system. Alternatively, cells that acquire the desired phenotype could also be separated by antibodies against extracellular epitopes or by the expression of transgenes including fluorescent proteins under the control of a cell type specific promoter. By way of example dopaminergic neurons could be isolated with fluorescent proteins expressed under the control of TH, DAT, Ptx3 or other promoters specifically used by dopaminergic neurons.

Methods of the invention may comprise additional negative or positive selection methods to enrich for neural stem, progenitor or precursor cells, or other stem or neural cells with the desired phenotype.

Negative selection may be used to enrich for DA neurons. Selective neurotoxins for non-DA neurons may be used, for instance 5-7-dihydroxytryptamine (to eliminate serotoninergic neurons), or antibodies coupled to saponin or a toxin or after addition of complement, for instance antibodies against GABA transporter (to eliminate GABAergic neurons). Methods of the invention may comprise additionally treating or contacting a neural stem, progenitor or precursor cell, or other stem or neural cell with a negative selection agent, preferably in vitro, e.g. by adding the negative selection agent to an in vitro culture containing the cell, or by culturing the cell in the presence of the negative selection agent. A negative selection agent selects against cell types other than the desired cell type(s). For example, where the invention relates to promoting, enhancing or inducing a dopaminergic neuronal phenotype, the negative selection agent may select against cells other than DA neurons and cells that develop into DA neurons such as stem cells and neural stem, precursor and progenitor cells. Thus, the negative selection agent may select against differentiated cells with a non-DA phenotype, such as non-DA neurons. The negative selection agent may reduce or prevent proliferation of and/or kill cells other than the desired cell type(s). The negative selection agent may be a selective neurotoxin that reduces the population of neurons other than DA neurons. For example, the negative selection agent may be 5-7-dihydroxytryptamine (to reduce serotoninergic neurons). The negative selection agent may be an antibody or antibody fragment specific for a non-DA neuron, wherein the antibody or antibody fragment (e.g. scFv or Fab) is coupled to saponin or to a toxin. For instance the antibody may be specific for GABA transporter (to reduce GABAergic neurons).

In methods of the invention, the neural stem, progenitor or precursor cell or other stem or neural cell may be grown in the presence of an antioxidant (e.g. ascorbic acid), low oxygen tension and/or a hypoxia-induced factor (e.g. HIF or erythropoetin).

The present invention further provides in various aspects and embodiments the use of an agent selected from a Wnt ligand, or nucleic acid encoding a Wnt ligand, or a synthetic Wnt ligand analogue, or a protein, nucleic acid or synthetic antagonist that inhibits or blocks the Wnt-inhibitory activities of soluble frizzed related proteins or dikkopfs or WIF, or a protein, nucleic acid or synthetic drug working to inhibit, block, enhance, switch or modulate one or more signalling components downstream of Wnts, in therapeutic methods comprising administering the Wnt ligand or encoding nucleic acid or other said agent to an individual to induce, promote or enhance dopaminergic neuron development in the brain by acting on either endogenous or on exogenously supplied stem, progenitor or precursor cells, or neuronal cells, and/or to inhibit or prevent loss or promote the survival or phenotypic differentiation or maturation, or neuritogenesis or synaptogenesis, or functional output, of dopaminergic neurons, e.g. in treatment of an individual with a Parkinsonian syndrome or Parkinson's disease. A Wnt ligand or encoding nucleic acid or other said agent may be administered in any suitable composition, e.g. comprising a pharmaceutically acceptable excipient or carrier, and may be used in the manufacture of a medicament for treatment of a neurodenerative disorder, Parkinsonian syndrome or Parkinson's disease. A Wnt ligand or encoding nucleic acid may be administered to or targeted to the central nervous system and/or brain.

The present invention extends in various aspects not only to a neuron produced in accordance with any one of the methods disclosed herein, but also a pharmaceutical composition, medicament, drug or other composition comprising such a neuron, stem, progenitor or precursor cell and/or a Wnt ligand, use of such a neuron, stem, progenitor or precursor cell or neuronal cell and/or Wnt ligand or composition in a method of medical treatment, a method comprising administration of such a neuron, stem, progenitor, precursor or neuronal cell and/or Wnt ligand or composition to a patient, e.g. for treatment (which may include preventative treatment) of Parkinson's disease or other (e.g. neurodegenerative) diseases, use of such a neuron or cell and/or Wnt ligand in the manufacture of a composition for administration, e.g. for treatment of Parkinson's disease or other (e.g. neurodegenerative diseases), and a method of making a pharmaceutical composition comprising admixing such a neuron or cell and/or Wnt ligand with a pharmaceutically acceptable excipient, vehicle or carrier, and optionally one or more other ingredients, e.g. a neuroprotective molecule, a neuroregenerative molecule, a retinoid, growth factor, astrocyte/glial cell, anti-apoptotic factor, or factor that regulates gene expression in stem, progenitor or precursor cells or neuronal cells or in the host brain. Such optional ingredients may render the neuron independent of its environment, i.e. such that its survival is not dependent on the presence of one or more factors or conditions in its environment. By way of example, the method of making a pharmaceutical composition may include admixing the neuron with one or more factors found in the developing ventral mesencephalon. The neuron may be admixed with GDNF and/or neurturin (NTN).

The present invention provides a composition containing a neuron, stem, progenitor or precursor cell or neuronal cell produced in accordance with the invention and/or a Wnt ligand, and one or more additional components. Pharmaceutical compositions according to the present invention, and for use in accordance with the present invention, may comprise, in addition to the neuron or cell, a pharmaceutically acceptable excipient, carrier, buffer, preservative, stabiliser, anti-oxidant or other material well known to those skilled in the art. Such materials should be non-toxic and should not interfere with the activity of the neuron. The precise nature of the carrier or other material will depend on the route of administration. The composition may include one or more of a neuroprotective molecule, a neuroregenerative molecule, a retinoid, growth factor, astrocyte/glial cell, or factor that regulates gene expression in stem, neural stem, precursor or progenitor cells or neuronal cells. Such substances may render the neuron independent of its environment as discussed above.

Liquid pharmaceutical compositions generally include a liquid carrier such as water, petroleum, animal or vegetable oils, mineral oil or synthetic oil. Physiological saline solution, tissue or cell culture media, dextrose or other saccharide solution or glycols such as ethylene glycol, propylene glycol or polyethylene glycol may be included.

The composition may be in the form of a parenterally acceptable aqueous solution, which is pyrogen-free and has suitable pH, isotonicity and stability. Those of relevant skill in the art are well able to prepare suitable solutions using, for example, isotonic vehicles such as Sodium Chloride, Ringer's Injection, or Lactated Ringer's Injection. A composition may be prepared using artificial cerebrospinal fluid.

The present invention extends to the use of a neuron produced in accordance with the invention and/or a Wnt ligand in a method of medical treatment, particularly the treatment of a medical condition associated with degeneration, damage to, the loss of, or a disorder in neuronal cells. Moreover, the invention may provide the use of a neuron of a specific phenotype and/or a Wnt ligand in the treatment of a condition, disease or disorder, which is associated with generation, damage to, or the loss of neurons of that phenotype. More particularly, the invention provides the use of a dopaminergic neuron and/or a Wnt ligand in the treatment of human Parkinson's disease. While the invention particularly relates to materials and methods for treatment of neurodegenerative diseases (e.g. Parkinson's disease), it is not limited thereto. By way of example, the invention extends to the treatment of degeneration in or damage to the spinal cord and/or cerebral cortex, or other regions of the nervous system containing Nurr1+ cells.

In methods of treatment in which the administered cell is a stem, progenitor or precursor that is capable of giving rise to two or more distinct neuronal phenotypes, the neuron, cell and/or Wnt ligand or composition may be introduced into a region containing astrocytes/glial cells which direct the differentiation of the cell to a desired specific neuronal fate. The cell and/or Wnt ligand or composition may for example be injected into the ventral mesencephalon where it may interact with Type 1 astrocytes/glial cells and be induced to adopt a dopaminergic phenotype. Alternatively or in addition, an implanted composition may contain a neuron or cell in combination with one or more factors which direct its development toward a specific neuronal fate as discussed above, e.g. with a Type 1 astrocyte/glial cell.

Cells may be implanted into a patient by any technique known in the art (e.g. Lindvall, O., (1998) Mov. Disord. 13, Suppl. 1:83-7; Freed, C. R., et al., (1997) Cell Transplant, 6, 201-202; Kordower, et al., (1995) New England Journal of Medicine, 332, 1118-1124; Freed, C. R., (1992) New England Journal of Medicine, 327, 1549-1555).

Administration of a composition in accordance with the present invention is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

The methods provided herein may be carried out using primary cells in vivo or in vitro or cell lines as a source material. The advantage of cells expanded in vitro is that there is virtually no limitation on the number of neurons which may be produced.

In order to ameliorate possible disadvantages associated with immunological rejection of transplanted cells, stem or progenitor or precursor cells may be isolated from a patient and induced to the desired phenotype. Cells may then be transplanted to the patient. Advantageously, isolated stem or progenitor or precursor cells may be used to establish cell lines so that large numbers of immunocompatible neuronal cells may be produced. A further option is to establish a bank of cells covering a range of immunological compatibilities from which an appropriate choice can be made for an individual patient. Stem, neural stem, precursor or progenitor cells or neuronal cells derived from one individual may be altered to ameliorate rejection when they or their progeny are introduced into a second individual. By way of example, one or more MHC alleles in a donor cell may be replaced with those of a recipient, e.g. by homologous recombination.

If cells derived from a cell line carrying an immortalizing oncogene are used for implantation into a patient, the oncogene may be removed using the CRE-loxP system prior to implantation of the cells into a patient (Westerman, K. A. et al Proc. Natl. Acad. Sci. USA 93, 8971 (1996)). An immortalizing oncogene which is inactive at the body temperature of the patient may be used.

In a further aspect the present invention extends to the use of a cell or neuron produced in accordance with the invention in a method of screening for an agent for use in the treatment of a neurodegenerative disease. The neuron may be a dopaminergic neuron. The neurodegenerative disease may be a Parkinsonian syndrome or Parkinson's disease. The agent may be a neuroprotective and/or neuroregenerative molecule and/or a developmental soluble signal and/or a factor or factors derived from ventral mesencephalic type one astrocytes or glial cells. The method may be carried out in vitro or in vivo.

The method may include:

(i) treating a neuron of the invention with a toxin for said neuron;
(ii) separating the neuron from the toxin;
(iii) bringing the treated neuron into contact with a test agent or test agents;
(iv) determining the ability of the neuron to recover from the toxin;
(v) comparing said ability of the neuron to recover from the toxin with the ability of the or an identical neuron to recover from the toxin in the absence of contact with the test agent(s).

The method may include:

(i) treating a neuron of the invention with a toxin for the neuron in the presence of a test agent or test agents;
(ii) determining the ability of the neuron to tolerate the toxin;
(iii) comparing said ability of the neuron to tolerate the toxin with the ability of the or an identical neuron to tolerate the toxin in the absence of contact with the test agent(s).

The toxin may be 6-hydroxydopamine, 5,7-dihydroxytryptamine or 1-methyl-4-phenyl-1,2,3,6-tetrahydropyridine (MPTP), proteasome inhibitors, including lactacystin, or pesticides, including rotenone, all of which lead to the death of catecholaminergic neurons and experimentally reproduce features of Parkinson's disease. The ability of the neuron to recover from or tolerate the toxin may be determined by any method known to those skilled in the art, for example by monitoring cell viability, (e.g. by cell counting, e.g. by the TUNEL technique), by monitoring morphology, (e.g. sprouting, axonal elongation and/or branching), and/or by monitoring biochemistry, (e.g. TH activity, e.g. neurotransmitter uptake/release/content).

Stem, neural stem, precursor, progenitor or neural cells which may be used in the present invention include C17.2 (Snyder, E. Y. et al. Cell 68, 33 51 (1992)) and the H6 human cell line (Flax et al. Nature Biotech 16 (1998)). Further examples are listed in: Gage et al. 1995, and Gotlieb 2002).

While the present discussion has been made with reference to neural stem cells or neural progenitor or precursor cells, the methods provided herein may be applied to the induction of neuronal fates in other stem, progenitor or precursor cells. Examples of such cells include stem cells associated with non-neural systems. The methods may be applied to stromal or hematopoietic stem cells and/or proliferative cells from the epidermis. Hematopoietic cells may be collected from blood or bone marrow biopsy. Stromal cells may be collected from bone marrow biopsy. Epithelial cells may be collected by skin biopsy or by scraping e.g. the oral mucosa. Since a neuronal phenotype is not a physiological in vivo fate of these stem, progenitor or precursor cells, the inductive process may be referred to as trans-differentiation, or de-differentiation and neural re-differentiation. A method of inducing such cells to a neuronal fate may include the use of antisense regulators to genes associated with non-neuronal phenotypes, i.e. to suppress and/or reverse the differentiation of these cells toward non-neuronal fates.

The methods of the present invention may be applied to stem cells not committed to a neural fate. They may be applied to stem cells which are capable of giving rise to two or more daughter stem cells associated with different developmental systems. Examples of these stem cells are embryonic stem cells, hematopoietic stem cells, proliferative cells from the epidermis, and neural stem cells.

As discussed above, the present disclosure demonstrates that dopaminergic neurons can be generated from stem or progenitor or precursor cells by a process requiring expression of Nurr1 above basal levels in combination with Wnt ligand and/or one or more factors derived from ventral mesencephalic type 1 astrocytes or glial cells.

In various further aspects the present invention is concerned with provision of assays and methods of screening for a factor or factors which enhance induction of a dopaminergic fate in a neural stem or progenitor or precursor cell or enhance dopaminergic induction or differentiation in a neuronal cell expressing Nurr1 above basal levels and treated with Wnt ligand, and with a factor or factors identified thereby.

The invention provides a method of screening for a factor or factors able, either alone or in combination, to enhance, increase or potentiate induction of a dopaminergic fate in a stem, neural stem or progenitor or precursor cell or neuronal cell expressing Nurr1 above basal levels in the presence of Wnt ligand. A further aspect of the present invention provides the use of a stem, neural stem or progenitor or precursor cell or neuronal cell expressing Nurr1 above basal levels and in the presence of Wnt ligand in screening or searching for and/or obtaining/identifying a factor or factors which enhance induction of a dopaminergic fate in such a stem or progenitor or precursor cell or neuronal cell.

A method of screening may include:

(a) bringing a test substance into contact with a stem, neural stem or progenitor or precursor cell or neuronal cell expressing Nurr1 above basal levels in the presence of Wnt ligand, which contact may result in interaction between the test substance and the cell; and
(b) determining interaction between the test substance and the cell.

A method of screening may include bringing a test substance into contact with a membrane fraction, soluble fraction or nuclear fraction derived from a stem, neural stem or progenitor or precursor cell or neuronal cell expressing Nurr1 above basal levels in the presence of Wnt ligand and determining interaction between the test substance and the fraction. The preparation of these fractions is well within the capabilities of those skilled in the art.

Binding or interaction may be determined by any number of techniques known in the art, qualitative or quantitative. Interaction between the test substance and the stem or progenitor or neuronal cell may be studied by labeling either one with a detectable label and bringing it into contact with the other which may have been immobilised on a solid support, e.g. by using an antibody bound to a solid support, or via other technologies which are known per se including the Biacore system.

A screening method may include culturing a stem, neural stem or progenitor or precursor cell or neuronal cell in the presence of a test substance or test substances and analyzing the cell for differentiation to a dopaminergic phenotype, e.g. by detecting a marker of the dopaminergic phenotype as discussed herein. Tyrosine hydroxylase (TH) is one marker of the dopaminergic phenotype.

Any of the substances screened in accordance with by the present invention may be a natural or synthetic chemical compound.

A screening method may include comparing Type 1 astrocytes or early glial cells of the ventral mesencephalon with neural cells (e.g. astrocytes) which are unable to induce a dopaminergic fate in stem, neural stem or progenitor or precursor cells expressing Nurr1 above basal levels in the presence of Wnt ligand. The comparison may for example be between Type 1 astrocytes or early glial cells during development of the ventral mesencephalon and Type 1 astrocytes or early glia from other neural locations.

A screening method involving astrocytes or early glial cells may employ immortalized astrocytes or immortalized glial cells. It may involve astrocyte cell lines or glial cell lines, e.g. astrocyte or glial mesencephalic cell lines. Such cell lines provide a homogenous cell population.

A screening method may employ any known method for analyzing a phenotypic difference between cells and may be at the DNA, mRNA, cDNA or polypeptide level. Differential screening and gene screening are two such techniques. A substance identified by any of the methods of screening described herein may be used as a test substance in any of the other screening methods described herein.

A screening method may employ a nucleic expression array, e.g. a mouse cDNA expression array. In this approach, an array of different nucleic acid molecules is arranged on a filter, quartz or another surface, e.g. by cross-linking the nucleic acid to the filter. A test solution or extract is obtained and the nucleic acid within it is labeled, e.g. by fluorescence. The solution or extract is then applied to the filter or genechip. Hybridisation of the test nucleic acid to nucleic acid on the filter or genechip is determined and compared to the hybridisation achieved with a control solution. A difference between the hybridisation obtained with the test and control samples is indicative of a different nucleic acid content. For further information on nucleic acid arrays, see Clontech website (e.g. www.clontech.com) or Affymetrix website (e.g. www.affymetrix.com), findable using any available web browser.

Screening methods are described here with reference to Nurr1 expressed above basal levels, but the disclosure also extends to all nuclear receptors of the Nurr1 subfamily e.g. Nor-1 and NGFI-B. Thus, Nurr1 is described by way of example and not by way of limitation. In any method of the invention, a nuclear receptor of the Nurr1 subfamily, including Nurr1 or any other receptor e.g. Nor-1 or NGFI-B, may be expressed above basal levels in the cell.

A screening method may include comparing stem or progenitor or precursor or neural cells with stem or progenitor or precursor cells or neural cells which express Nurr1 above basal levels in the presence of Wnt ligand, e.g. to identify target genes of Nurr1 and/or a factor or factors which enhance the proliferation and/or self-renewal and/or the differentiation and/or survival and/or promote the acquisition or the induction of a dopaminergic fate and/or induce dopaminergic neuron development in stem, neural stem, precursor, progenitor or neural cells and/or enhance dopaminergic induction or differentiation in a neuronal cell expressing Nurr1 above basal levels in the presence of Wnt ligand. Once the target gene(s) and/or factor(s) have been identified they may be isolated and/or purified and/or cloned and used in further methods.

A screening method may include purifying and/or isolating a substance or substances from a mixture. The method may include determining the ability of one or more fractions of the mixture to interact with a stem cell, neural stem cell or neural progenitor or precursor cell or neural cell expressing Nurr1 above basal levels in the presence of Wnt ligand, e.g. the ability to bind to and/or promote the proliferation and/or self-renewal and/or enhance induction, acquisition, differentiation or development of a dopaminergic phenotype or fate in such a stem, neural stem, precursor, progenitor or neural cell. The purifying and/or isolating may employ any method known to those skilled in the art.

A screening method may employ an inducible promoter operably linked to nucleic acid encoding a test substance. Such a construct is incorporated into a host cell and one or more properties of that cell under the permissive and non-permissive conditions of the promoter are determined and compared. The property determined may be the ability of the host cell to induce a dopaminergic phenotype in a stem, neural stem, precursor, progenitor or neural cell expressing Nurr1 above basal levels in the presence of Wnt ligand. A difference in that ability of the host cell between the permissive and non-permissive conditions indicates that the test substance may be able, either alone or in combination, to enhance proliferation and/or self-renewal and/or induction of a dopaminergic fate and/or dopaminergic differentiation, survival or development in a stem, neural stem or progenitor or precursor cell or enhance dopaminergic induction or differentiation in a neuronal cell expressing Nurr1 above basal levels in the presence of Wnt ligand.

The precise format of any of the screening methods of the present invention may be varied by those of skill in the art using routine skill and knowledge.

A factor or factors identified by any one of the methods provided by the invention may be isolated and/or purified and/or further investigated. It may be manufactured.

In various further aspects, the invention further provides a factor identified by any one of the methods disclosed herein, a pharmaceutical composition, medicament, drug or other composition comprising such a factor (which composition may include a stem, neural stem or progenitor or precursor cell or neuron expressing Nurr1 above basal levels and Wnt ligand), use of such a factor to enhance induction and/or phenotypic differentiation or maturation and/or survival and/or neuritogenesis and/or synaptogenesis and/or functional output of dopaminergic neurons derived from stem, neural stem or progenitor or precursor cells expressing Nurr1 above basal levels in the presence of Wnt ligand, use of such a factor or composition in a method of medical treatment, a method comprising administration of such a factor or composition to a patient, e.g. for treatment (which may include preventative treatment) of a medical condition associated with degeneration, damage to, loss of, or a disorder in or affecting dopaminergic neurons, e.g. for treatment of Parkinson's disease or another neurodegenerative disease, use of such a factor in the manufacture of a composition, medicament or drug for administration, e.g. for treatment of Parkinson's disease or other (e.g. neurodegenerative diseases), and a method of making a pharmaceutical composition comprising admixing such a factor with a pharmaceutically acceptable excipient, vehicle or carrier, and optionally other ingredients.

In a related aspect, the present invention provides a method of screening for a substance which modulates the ability of a Wnt ligand to induce a dopaminergic fate in stem, neural stem, precursor or progenitor cells or enhance dopaminergic induction or differentiation in a neuronal cell expressing Nurr1 above basal levels.

Thus, the method may screen for a substance which modulates the ability of a Wnt ligand to induce proliferation, self renewal, dopaminergic development, differentiation, maturation and/or acquisition of a dopaminergic fate in stem, neural stem, precursor, progenitor or neural cells expressing Nurr1 above basal levels.

Such a method may include one or more of:

(i) providing stem, neural stem, progenitor, precursor or neural cells which express Nurr1 above basal levels in the presence of a Wnt ligand and one or more test substances;
(ii) analysing the proportion of such cells which adopt a dopaminergic fate or phenotype and/or respond to Wnts;
(iii) comparing the proportion of such cells which adopt a dopaminergic fate with the number of such cells which adopt a dopaminergic fate or phenotype and/or respond to Wnts in comparable reaction medium and conditions in the absence of the test substance or test substances. A difference in the proportion of dopaminergic neurons between the treated and untreated cells is indicative of a modulating effect of the relevant test substance or test substances.

Such a method of screening may include:

(i) bringing stem, neural stem, precursor or progenitor cells or neuronal cells which express Nurr1 above basal levels into contact with a Wnt ligand in the presence of one or more test substances;
(ii) analysing the proportion of stem, neural stem, precursor or progenitor cells or neuronal cells which adopt a dopaminergic fate or phenotype and/or respond to Wnts;
(iii) comparing the proportion of stem, neural stem, precursor, progenitor or neural cells which adopt a dopaminergic fate or phenotype and/or respond to Wnts with the number of stem, precursor, progenitor or neural cells which adopt a dopaminergic fate or phenotype and/or respond to Wnts in comparable reaction conditions in the absence of the test substance or test substances.

Such screening methods may be carried out on cells in vivo in comparable or identical non-human animals, or in vitro or in culture.

Following identification of a substance which modulates Wnt or inductive activity, the substance may be investigated further. It may be manufactured and/or used in the preparation, i.e. manufacture or formulation, of a composition such as a medicament, pharmaceutical composition or drug. Any of substance tested for its modulating activity may be a natural or synthetic chemical compound.

Aspects and embodiments of the present invention will now be illustrated, by way of example, with reference to the accompanying figures. Further aspects and embodiments will be apparent to those skilled in the art. All documents mentioned in this specification are incorporated herein by reference.

### BRIEF DESCRIPTION OF THE FIGURES

FIG. 1 shows that Wnt ligands are differentially expressed in the developing midbrain.

FIG. 1a shows results of real time PCR analysis that revealed that Wnt-1 is expressed at high levels in the ventral and the dorsal midbrain, while Wnt-3a expression predominates in the dorsal midbrain and Wnt-5a in the ventral midbrain.

FIG. 1b, FIG. 1c, FIG. 1d and FIG. 1e, show results of in situ hybridization that showed that within the ventral midbrain, the domains of Wnt-1 (FIG. 1d) and Wnt-5a expression (FIG. 1e) coincides with those of Nurr1 (FIG. 1c) and Tyrosine hydroxylase (TH) (FIG. 1b), that label dopaminergic precursors and dopaminergic neurons. FIG. 1f shows results of in situ hybridization demonstrating that Type 1 astrocytes isolated from postnatal day 1 ventral midbrain (VM) express significantly higher levels of Wnt-5a mRNA than dorsal midbrain (DM) or cerebral cortex (CC). *p<0.05; **p<0.001; ***p<0.0001 compared to E10.5 for every brain region by one-way ANOVA with Fisher's post hoc test.

FIG. 2 shows that Wnt-1 and Wnt-5a, but not Wnt-3a, increased the number of dopaminergic neurons (FIG. 2a, FIG. 2b and FIG. 2c) and proliferating clusters containing dopaminergic neurons (FIG. 2e and FIG. 2f) in rat E14.5 ventral mesencephalic (VM) cultures. FIG. 2a and FIG. 2e show dose dependency; FIG. 2b and FIG. 2f show time course analysis and FIG. 2c shows comparison of the effect of Wnt ligands with control (N2), glial cell line derived neurotrophic factor (GDNF), fibroblast growth factor2 and 8 (FGF-2 and -8), VM type 1 astrocytes (T1A) and control purified media (CP). The results indicate that the partially purified Wnts are active, stable and can be as efficient as VM T1A at increasing the number of dopaminergic neurons in culture. FIGS. 2b and c: *, **p<0.01 compared to N2 and CP, respectively; FIG. 2e: *p<0.01 compared to CP; by one-way ANOVA with Fisher's post hoc test. Tyrosine hydroxylase (TH) immunostained cultures, showed that Wnt-1 and Wnt-5a induced a very dramatic increase in the number of dopaminergic neurons outside or within dopaminergic clusters. A field is 3.14 mm2 and a well is 4 cm2.

FIG. 3 shows that Wnt-1 increased the proliferation of precursors and the total number of neurons, but unlike Wnt-5a, did not increase the proportion of dopaminergic precursors that acquired a dopaminergic phenotype. FIG. 3a shows that Wnt-1, but not Wnt-5a increased the expression of cyclin D1 mRNA. FIG. 3b shows that Wnt-1 and Wnt-3a induced a 3-fold increase in the proportion of Nurr1-immunostained cells that incorporated BrdU, while Wnt-5a and conditioned media from ventral mesencephalic type 1 astrocytes (VM T1A) increased BrdU incorporation in Nurr1+ cells to a lesser extent. FIG. 3d shows that Wnt-1 and Wnt-3 a increased the number of proliferating clusters that contained TuJ1-positive neurons. FIG. 3e shows that the proportion of dopaminergic neurons in proliferating clusters containing neurons did not change after Wnt-1 treatment, decreased by Wnt-3a, and was increased by treatment with Wnt-5a and VM T1A. FIG. 3f shows that only Wnt-1 increased the number of individual Tuj1-positive neurons outside proliferating clusters. FIG. 3g shows that despite the increase in the number of neurons outside the clusters, Wnt-1 did not increase the proportion of dopaminergic neurons. Instead, those treatments that did not change the numbers of Tuj1-positive neurons, either decreased (Wnt-3a) or increased (Wnt-5a or VM T1A) the proportion of dopaminergic neurons. *p<0.05; **p<0.001; ***p<0.0001 compared to control purified media (CP) by one-way ANOVA with Fisher's post hoc test.

FIG. 4 shows that Wnt-5a is the most efficient factor at inducing a dopaminergic phenotype in Nurr1+ cells. FIG. 4a shows results of double tyrosine hydroxylase (TH) and Nurr1 immunohistochemistry that revealed that Wnt-5a or VM T1A treatment increased the proportion of Nurr1 expressing cells in the VM that acquired a dopaminergic phenotype from 50% in control conditions to 90%. Instead, the two most potent factors at inducing proliferation, Wnt-1 and Wnt-3a, were less efficient than Wnt-5a (Wnt-1) or even decreased the proportion of dopaminergic cells from 50% to 30% (Wnt-3a). FIG. 4b shows that, similarly, Wnt-5a and VM T1A were the most efficient treatments at promoting the acquisition of a dopaminergic phenotype in Nurr1-expressing E13.5 cortical precursor cultures. Wnt-1 had a much lower effect than Wnt-5a and Wnt-3a or cortical type 1 astrocytes (CTX T1A), which did not change the proportion of Nurr1+ cells that expressed TH. *p<0.05; **p<0.001; ***p<0.0001 compared to control purified media (CP) by one-way ANOVA with Fisher's post hoc test.

FIG. 5 illustrates that Wnt signaling is required for the development of dopaminergic neurons.

FIG. 5a shows that E 13.5 VM neurospheres expanded with FGF8 differentiated in 5-7 days into glial and neuronal lineages and gave rise to dopaminergic neurons in 12% of the spheres. In contrast, addition of Fz8 CRD to the culture media decreased the number of neurospheres containing dopaminergic neurons, as compared to control (CM=CP). Similarly, treatment of E14.5 VM precursor cultures (FIG. 5b, FIG. 5c and FIG. 5d) with conditioned media from a Fz8 CRD overexpressing fibroblast decreased the proportion of Nurr1 immunoreactive cells that acquired tyrosine hydroxylase expression in control conditions (CM, FIG. 5b), or after treatment with either conditioned media from ventral mesencephalic type 1 astrocytes (VM T1A, FIG. 5c) or Wnt-5a (FIG. 5d). *p<0.05; ***p<0.0001 compared to control (N2) or conditioned media (CM); #p<0.05; ###p<0.0001 compared to VM T1A or Wnt-5a by one-way ANOVA with Fisher's post hoc test. FIG. 5f, Model of the mechanisms by which Wnt-1, -3a and -5a regulate the development of VM dopaminergic neurons. Wnt1, probably derived from the midbrain-hindbrain organizer, controls the proliferation of Nurr1-expressing precursors and increases the number of VM neurons. Wnt-5a, which is expressed by VM astroglial cells, specifically increases the number of VM dopaminergic neurons by regulating the induction of a dopaminergic phenotype in Nurr1-expressing precursors. Finally, Wnt-3a, which is mainly expressed in the dorsal midbrain, enhanced the proliferation and/or self renewal of Nurr1-expressing precursors and decreased the proportion of neurons that acquire a dopaminergic phenotype. Question marks indicate that the precise cell source of Wnt-1 and Wnt-3a is unknown. Note that the size of the arrows correlates with the intensity of the effects.

FIG. 6 shows that Wnts differentially control the development of dopaminergic neurons by regulating precursor proliferation and the acquisition of a DA phenotype. Wnt-5a, but not Wnt-1, upregulated the expression of Ptx3 mRNA (A) and c-ret (B), and maintained the expression of GFR□1 (C) and NCAM (D) at 3 days in vitro, as assessed by real time RT-PCR.

FIG. 7. Wnt-1 regulates the expression of cyclin D1 and the cell cycle inhibitors p27 and p57, increased the proliferation of VM precursors and specifically increases the number of TH neurons. Real time RT-PCR showed that Wnt-1, but not Wnt-5a, increased the expression of cyclin D3 mRNA (A) and decreased the expression of the cell cycle inhibitors p27 (B) and p57 (C) at 3 days in vitro. D, Wnt-1 and Wnt-3a, but not Wnt-5a, increased the proliferation of VM precursors at 3 days in vitro. E, Increasing units of partially purified Fz8-CRD, a Wnt blocking reagent, reduced the number of TH+ neurons in VM cultures dose-dependently after 3 days in vitro, indicating that endogenous Wnts are required for DA development. F, The increase in the number of TH+ neurons by Wnt-1 was partially blocked by Fz8-CRD, suggesting that the effects of Wnts are specific. Statistics: *p<0.05; *p<0.01; **p<0.001; ***p<0.0001 compared to control and ###p<0.0001 compared Wnt treatment alone, by one-way ANOVA with Fisher's post hoc test (n=3-5, except Fz8-CRD, n=2-3). Concentrations: 10 units/μl of Wnts or CP and 5 units/μl of Fz8-CRD. A field is 3.14 mm2.

FIG. 8 shows expression of Wnts in the developing midbrain. Real time RT-PCR analysis revealed that Wnt-2 (8a), Wnt-4 (8b), Wnt7a (8c), and Wnt-7b (8d) transcripts predominate the ventral midbrain area at the time of birth of dopaminergic neurons.

FIG. 9 shows expression of Wnts in the developing CNS. Real time RT-PCR analysis showed Wnt-3 (9A), Wnt-6 (9B), Wnt-10b (9C), Wnt-11 (9D), and Wnt-16 (9E) expression more specific to the dorsal mesencephalic region and other areas of the CNS.

FIG. 10 shows that Wnt-2 and Wnt-7a increased the number of dopaminergic neurons in rat E14.5 ventral precursor cultures. Treatment with Wnt-2 and Wnt-7a increased both the total number of tyrosine hydroxylase positive neurons in culture (10A) and in Nurr1-expressing precursors (10B). Wnt-7a increased the proliferation of ventral precursors (10C) while Wnt-2 treatment resulted in a decreased amount of BrdU incorporation in primary cultures (10C).

FIG. 11 shows different effects of Wnts on cell cycle regulators. Wnt-7a increased cyclin D1 mRNAs while downregulating the cdk inhibitors p27 and p57 (11A, 11D and 11E). Increases in cyclin D2 expression along with increases of p27 and p57 mRNAs were observed upon Wnt-2 treatment (11B, 11D and 11E)

### EXPERIMENTAL

Specifically incorporated by reference herein are the experimental results set out in WO00/66713 demonstrating proliferation and/or self-renewal of dopaminergic precursors and induction of dopaminergic neurons in stem, neural stem, precursor or progenitor cells expressing Nurr1, in the presence of type 1 astrocytes or glial cells, and demonstrating additional results obtained when contacting such cells with additional factors, such as FGFs (e.g. FGF8) or retinoids.

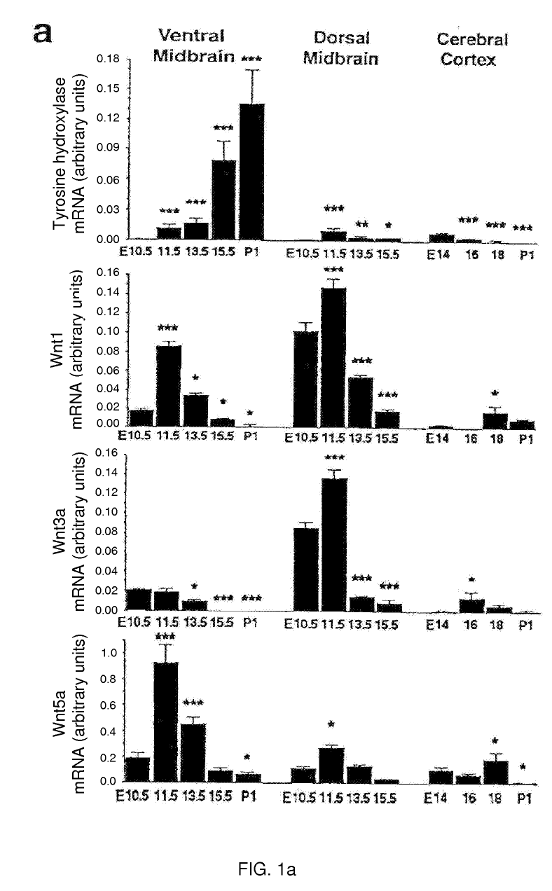

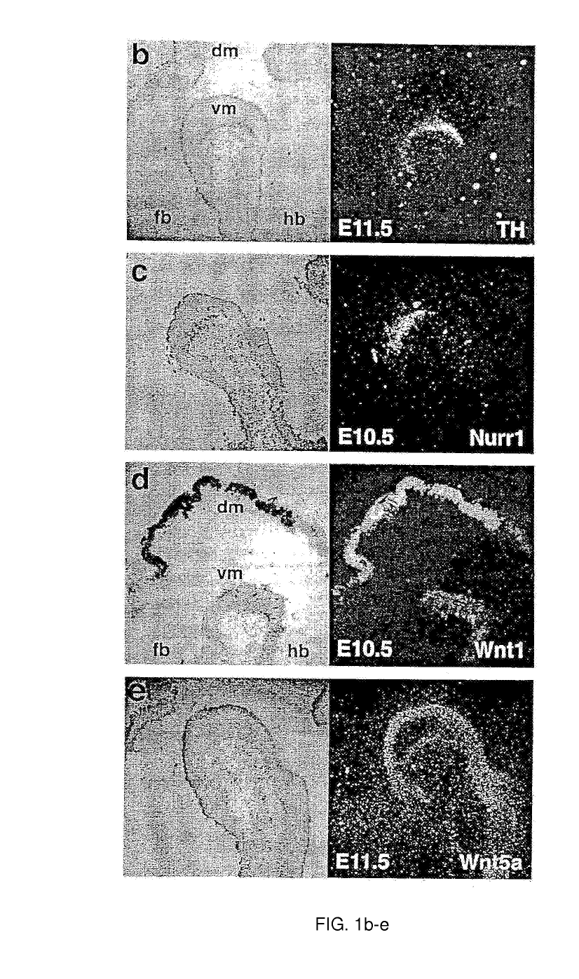

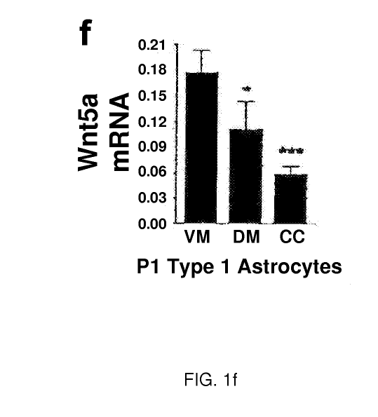

The expression of Wnts, Nurr1 and tyrosine hydroxylase (TH) was examined in the developing ventral midbrain and compared to dorsal midbrain (DM) and cerebral cortex from E10.5 to P1. Real time RT-PCR and ISH showed that TH message was clearly detected at E11.5 in the mouse and was first detected at E11.5 in the rat ventral midbrain (FIG. 1a and FIG. 1b), coinciding with the birth of dopaminergic neurons (Foster et al. 1988), which takes place one day after the onset of Nurr1 expression (FIG. 1c) and continues until E16 in the rat VM. Wnt-1 and Wnt-5a were the Wnts with the highest levels of expression in the mouse ventral midbrain (FIG. 1d and FIG. 1e), which peaked at E11.5 in the rat (FIG. 1a). A similar peak was detected at E11.5 in the dorsal midbrain for Wnt-1 and Wnt-3a, but not for Wnt-5a, consistent with a predominant role of Wnt-5a in ventral development (Saneyoshi et al., 2002). Moreover, real time RT-PCR analysis of Wnt expression in purified type 1 astrocytes showed that Wnt-5a was expressed in ventral mesencephalic astrocytes and that the levels of expression were significantly higher in ventral mesencephalic than in dorsal midbrain or cerebral cortex astrocytes (FIG. 1f).

In addition to Wnt-1, 3a and 5a mRNA expression, which are expressed at high levels in the developing midbrain, a more detailed analysis performed by real time RTPCR revealed that Wnt-2 and -7a mRNAs are expressed at intermediate levels in the midbrain, and that Wnt-7b and Wnt-16 are expressed at low levels in the midbrain. All of these ligands were expressed at higher levels in the ventral than the dorsal midbrain, suggesting a possible role of Wnt-2, -7a, -7b, and -16 in ventral midbrain development. We also found that Wnt-4, -6, -10b, and -11 were expressed at low levels in the midbrain. Wnt 3, 13 and -2b were expressed at very low levels in the midbrain and Wnt-5b, -8a/d, -10a, and -15 were detected in the midbrain at background level.

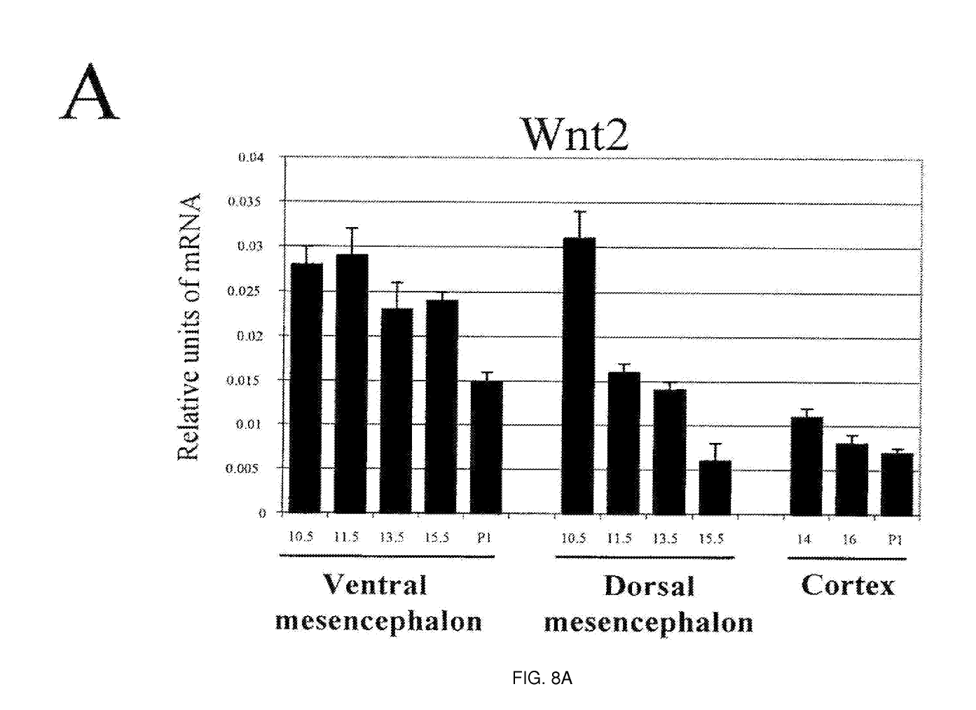

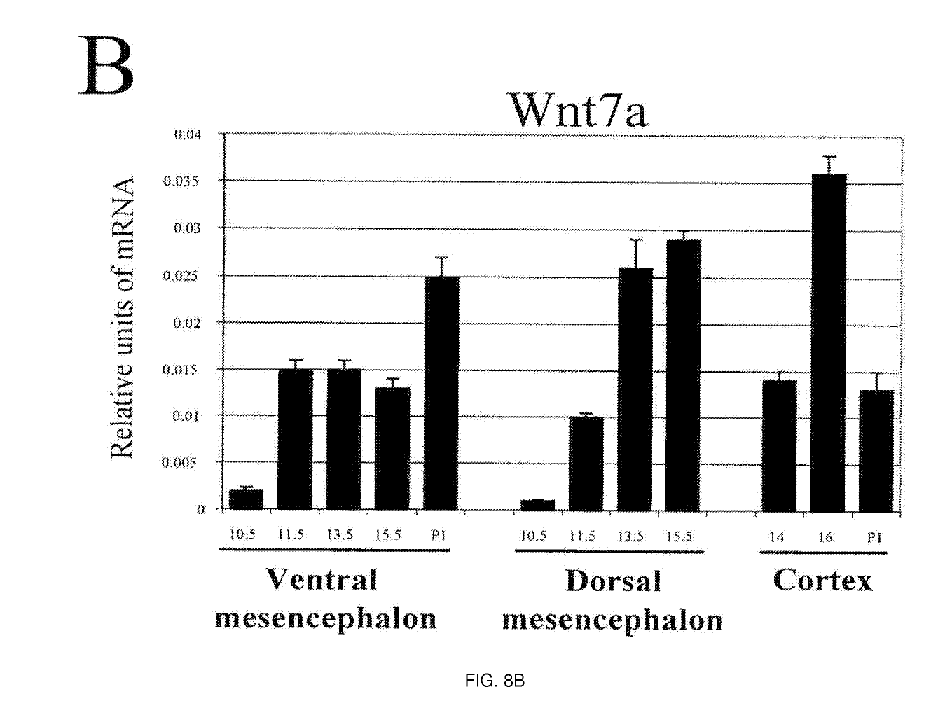

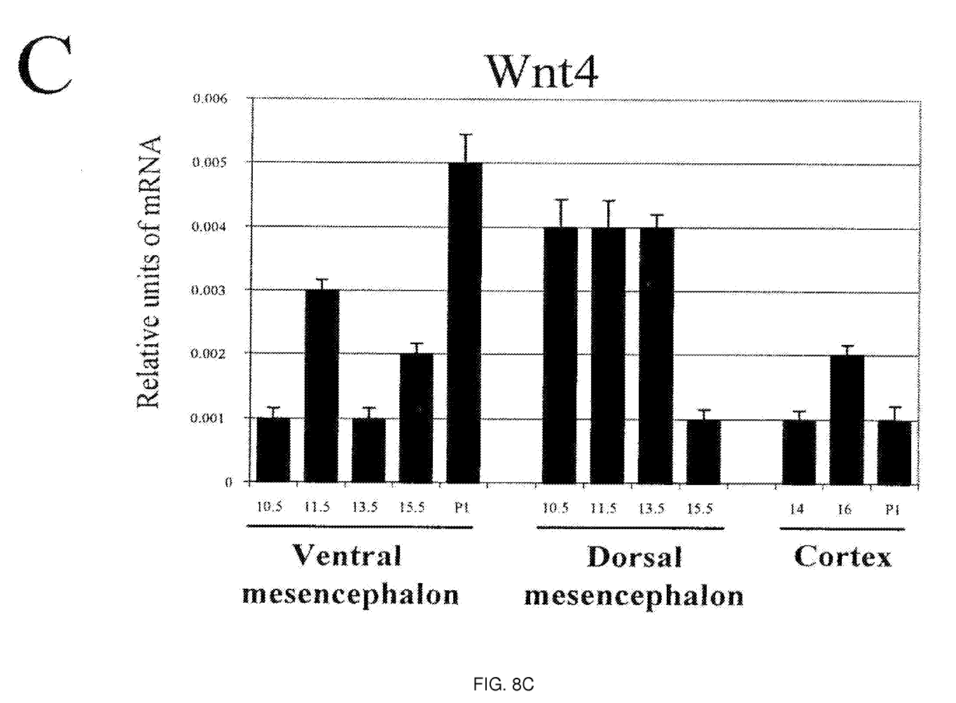

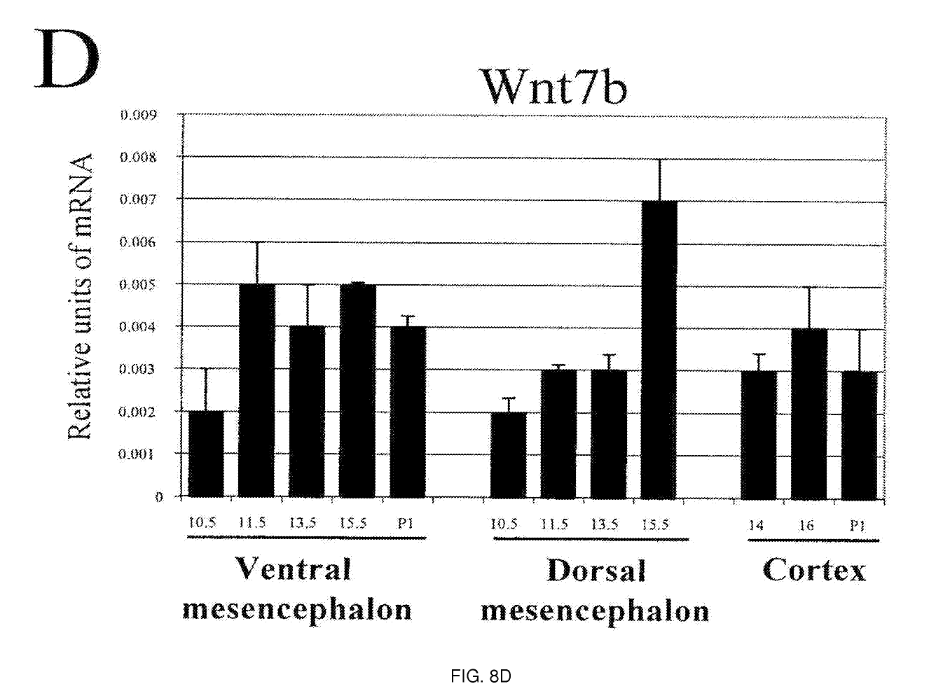

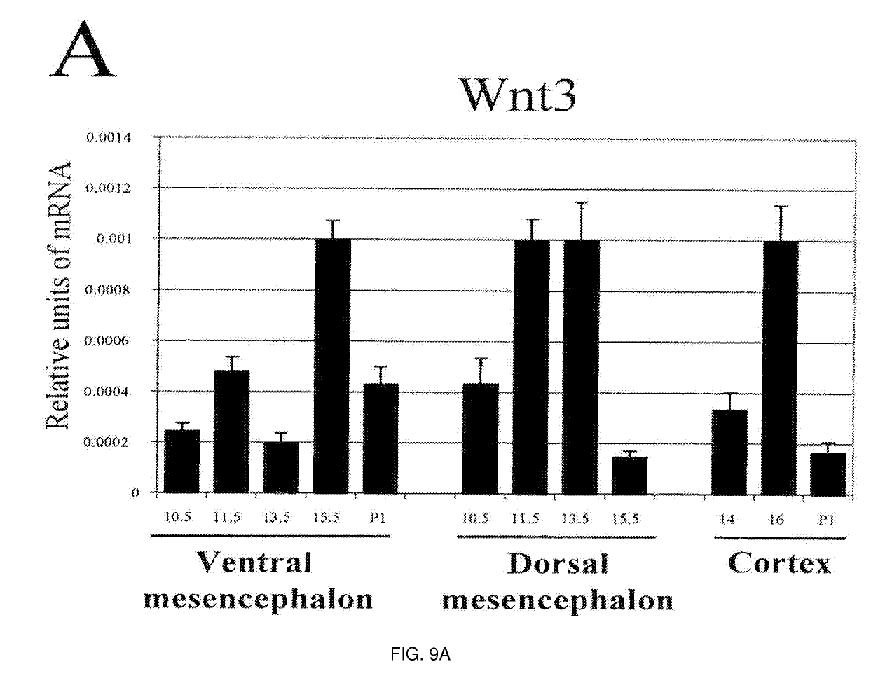

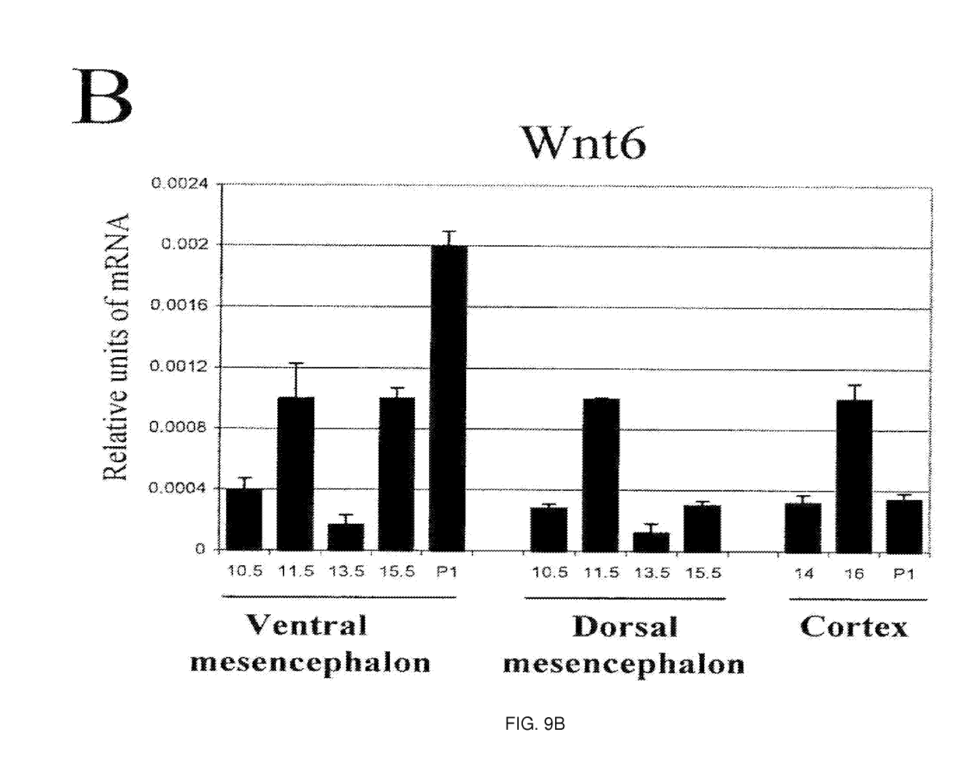

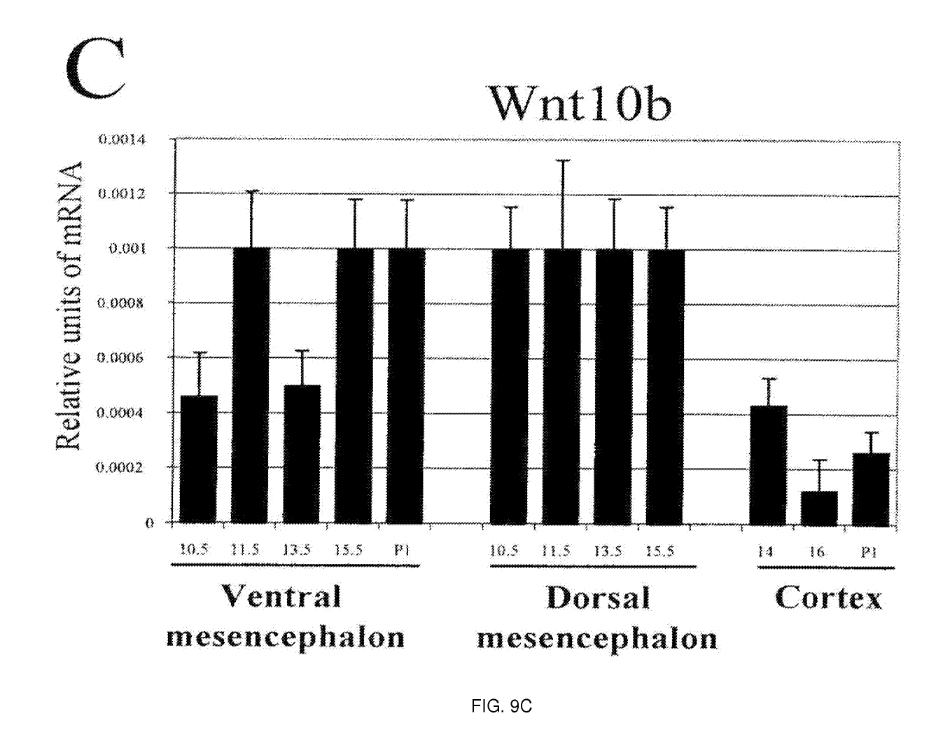

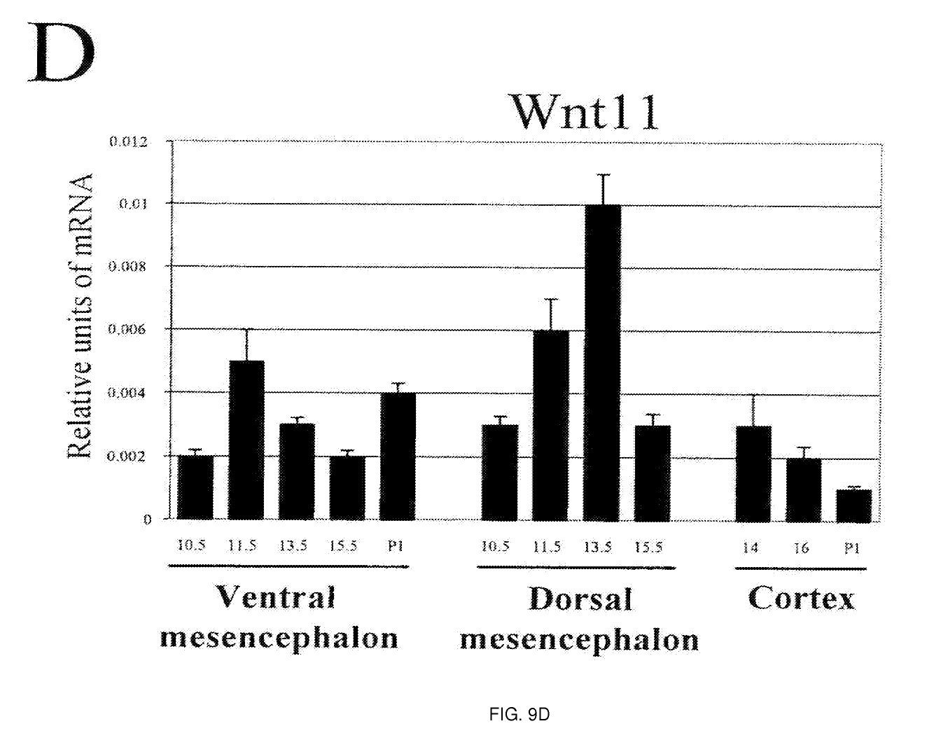

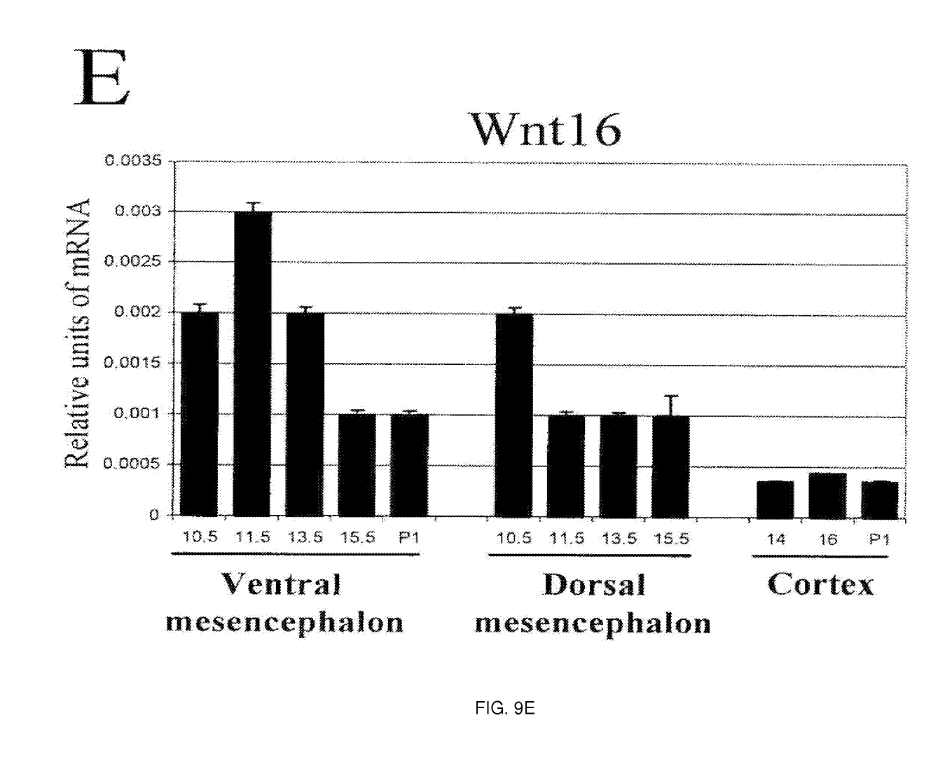

Birth of DA neurons in the VM is known to occur at embryonic day 11.5 (E11.5) in rat. We examined the expression patterns of the entire family of Wnt proteins in the developing rat brain and extended our study to Wnts that are expressed in the VM at significant levels by the time of birth of DA neurons. Of the Wnts analyzed, Wnt-2, -4, -7a, -7b, displayed higher levels of expression in the VM than in the DM at E11.5 and/or peaked in the VM at the time of birth of DA neurons, at E11.5 (FIGS. 8A, 8B, 8C and 8D). Of these, Wnt-2 and Wnt-7a were expressed in the VM at higher levels (FIGS. 8A and 8B respectively). In contrast, no expression of Wnts-5b, -8a/8d, -10a, and -15 was observed in any of the brain regions analyzed. Transcripts of Wnt -2b/13, Wnt-3, Wnt-6, and Wnt-10b, and Wnt-11 were primarily restricted to the dorsal mesencephalic region but at low levels (FIGS. 9A, 9B, 9C and 9D). Albeit at low levels, transcripts of Wnt-16 were detected in both areas throughout embryonic development (FIG. 9E). These data reveal a dynamic pattern of expression among a family of closely related extracellular signaling molecules. Moreover, the spatial and temporal expression of these genes suggests that members of the Wnt family function quite differently throughout embryonic development. Thus, the expression profiles presented here indicated a possible role for Wnt-2, Wnt-4, Wnt-7a, and Wnt-7b in the development of DA neurons.

We examined the expression of β-catenin, a central signaling component of the Wnt canonical pathway, in DA precursor cells characterized at E10.5 in the mouse by the expression of the orphan nuclear receptor Nurr1. It was found that dopaminergic precursors in the ventral midbrain express high levels of β-catenin. Double immunohistochemistry showed that β-catenin is expressed in the same domain as Nurr1 at E10.5 in the mouse, providing indication that Wnt signaling and β-catenin stabilization takes place in VM DA precursor cells during normal development in vivo.

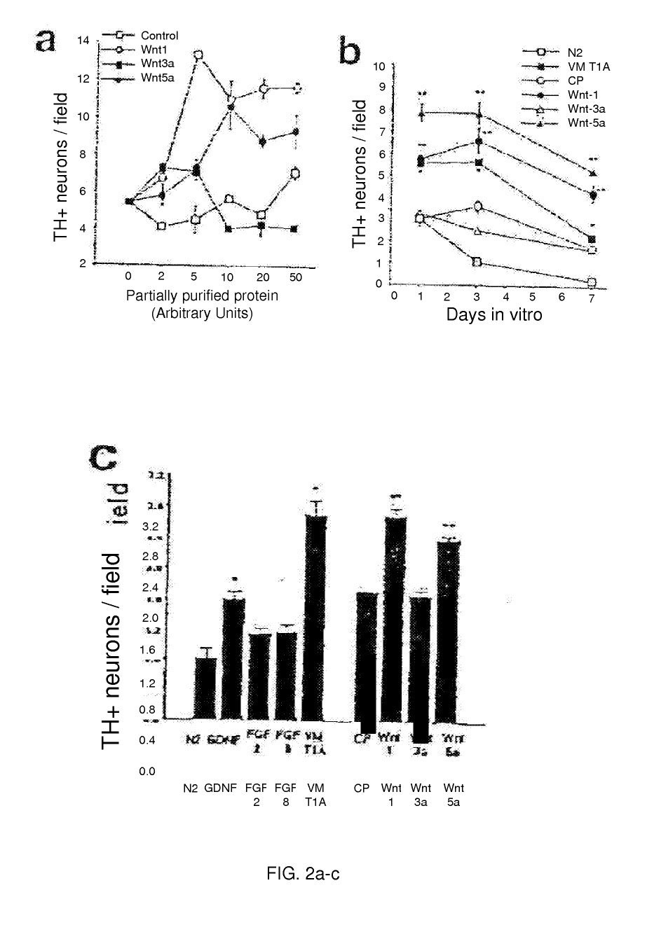

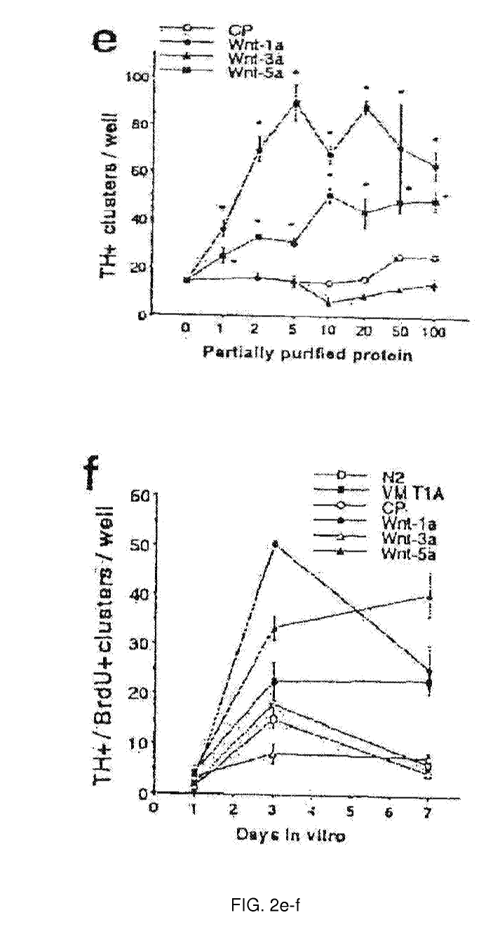

Partially purified conditioned media from stable fibroblast cell lines engineered to secrete Wnt ligands (Shimizu et al., 1997) was generated by size-exclusion-based filtration. After two sequential rounds of purification, an approximate 250-1500-fold increase in concentration was achieved, as compared to initial CM. Individual lots of partially purified Wnt ligands were normalized to each other based on density of combined Western blot product bands and expressed as arbitrary units, where 1 unit is equivalent to 1 μL of normalized partially purified product. Cultures of ventral mesencephala obtained from E14.5 rats were then treated with increasing concentrations of partially purified Wnt-1, -3a, -5a, or control (CP) media for 3 days (FIG. 2A). Treatment with Wnt-1 or 5a resulted in robust dose-response increases in TH+ cell numbers, while Wnt-3a or CP treatment produced no effect at doses up to 100 units and control media produced little or no effect up to 50 units (μL), suggesting that increases in TH+ cell number observed with Wnt-1 or -5a treatment were specific to those ligands. Interestingly, the effects of 10 units of Wnt-1 or -5a were detected as early as after 1 day in vitro, were higher than that of CP and Wnt-3a at 3 days and started to diminish after 7 days (FIG. 2B). Our results provided an indication that partially purified Wnts are fully stable for at least three days and that their effects are maintained throughout one week. Furthermore, these increases in TH+ cell numbers seemed biologically relevant (FIG. 2C), as they exceeded increases produced by known dopaminotrophic molecules after 3 days, including GDNF, FGF-2 or FGF-8, and roughly equaled the effects of VM T1A conditioned medium, which contains an inductive signal for Nurr1-expressing neural stem cells.

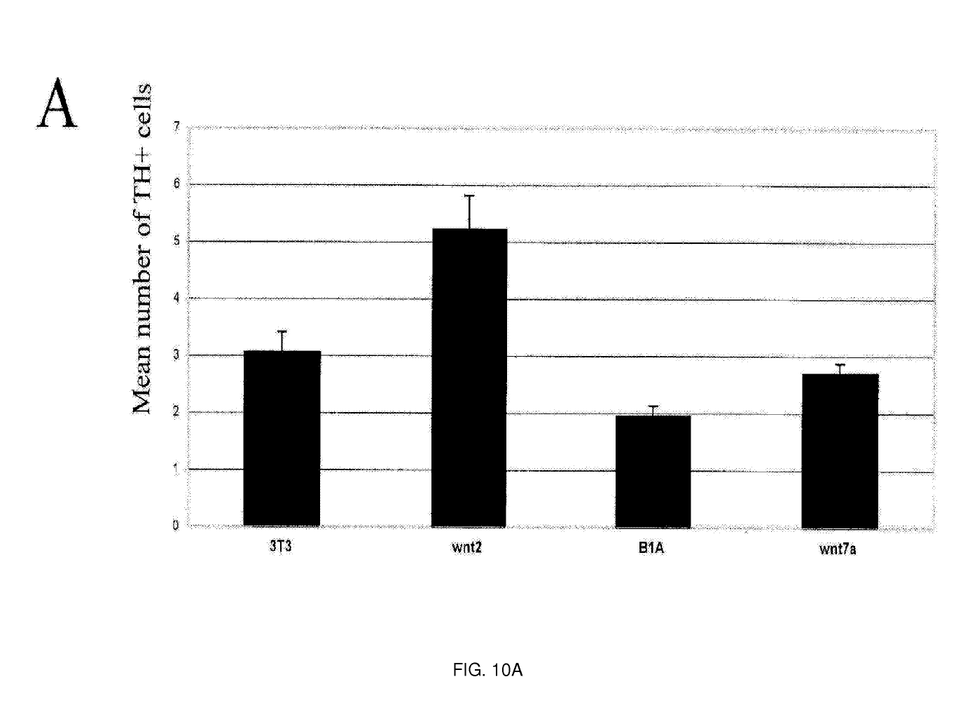

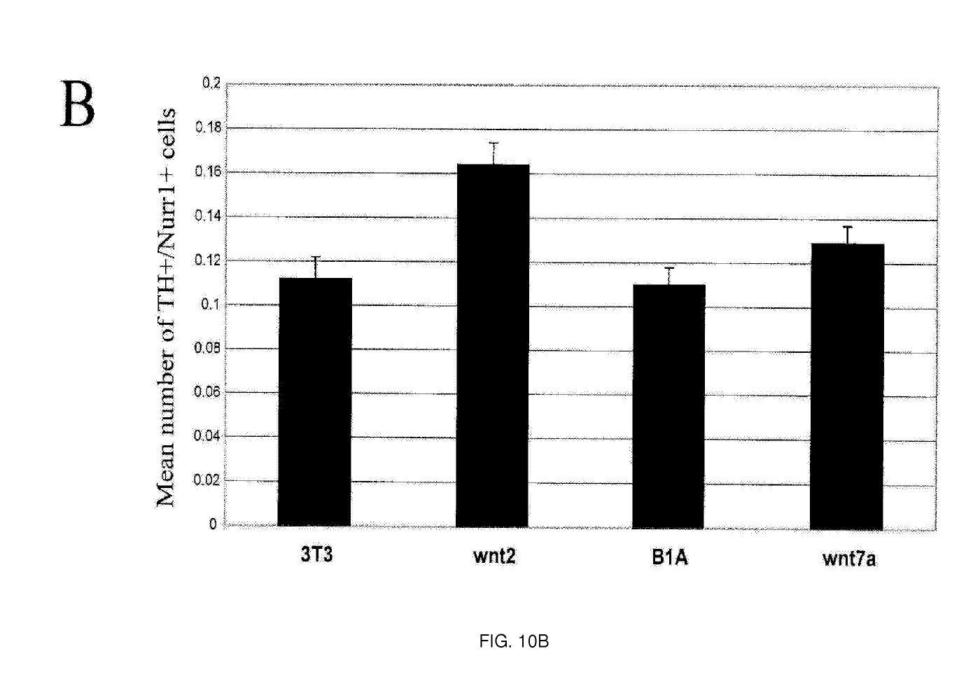

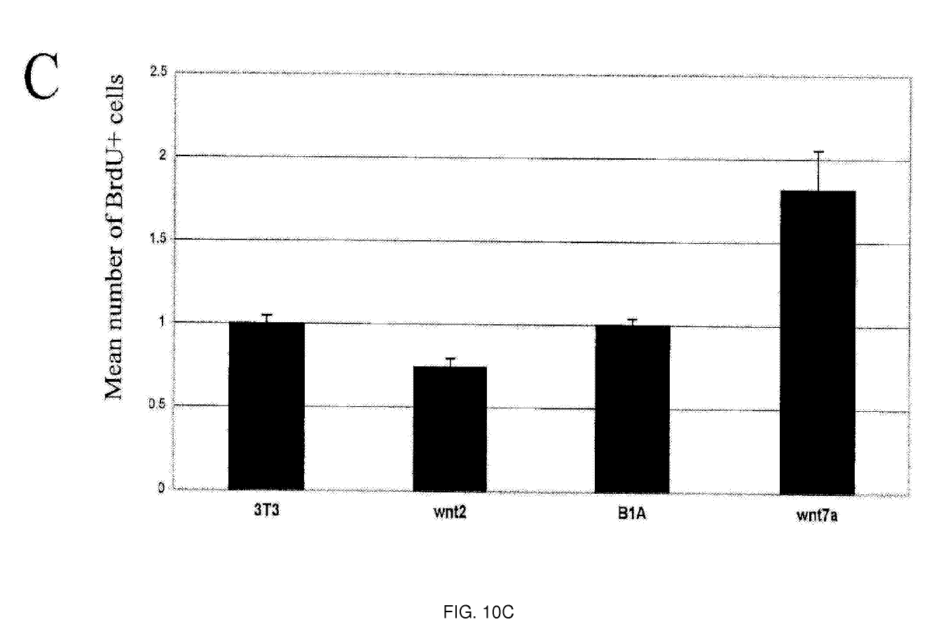

Conditioned media from Wnt-2 and Wnt-7a overexpressing fibroblast cell lines gave similar results to Wnt-1 and Wnt-5a partially purified media, and both Wnt-2 and Wnt-7a increased the number of TH+ neurons in E14.5 ventral mesencephalic precursor cultures, as compared to control fibroblast conditioned media. Upon treatment with partially purified Wnts, increases in TH immunoreactivity were observed for both Wnt-2 and Wnt-7a (FIG. 10A). Treatment with Wnt-7a resulted in modest TH+ increases compared to controls and other Wnts. In parallel experiments, treatment with Wnt-2 resulted in approximately a 2-fold increase in TH+ neurons. These data show that Wnt-2 and Wnt-7a treatment increase the number of TH+ neurons in VM precursor cultures. The same effects of Wnt-2 and Wnt-7a were observed when double immunohistochemistry using TH and Nurr1 markers were used (FIG. 10B), indicating that the effect exerted by these Wnts is on the Nurr1-expressing precursors.

Thus, our results indicate that members of the Wnt family of glycoproteins regulate very efficiently the number of dopaminergic neurons that develop from progenitor cells. Moreover, our data show that Wnt ligands have overlapping effects with regard to the regulation of TH cell number, suggesting that the function of some of the Wnts may be to some extent redundant.

Wnt-ligand treatment also induced the appearance of large spherical proliferative clusters. These clusters were initially small and increased in size over time in culture. Clusters of TH+ neurons, greater than 5 cell diameters and containing one or more TH+ neurons, were counted at various times in vitro. Strikingly, 10 units of Wnt-1 and -5a treatment, but not Wnt-3a, produced a 3-8 fold increase in the number of these clusters observed at 7 days in vitro (FIG. 2E and FIG. 2F). Moreover, the clusters that did appear in response to the Wnt ligands were larger and the vast majority consisted of almost entirely of TH+ neurons. On the other hand, control and Wnt-3a treated cultures generally contained smaller spheres. Interestingly, virtually all clusters and a significant number of isolated cells in the cultures were BrdU positive after an acute BrdU pulse prior to fixation (FIG. 2f). Thus, mitotic spheres could derive from the recruitment of dopaminergic precursors into the cell cycle, from the induction or differentiation of precursor (and/or less committed, still proliferating) cells into TH positive cells.

Because deletion of Wnt-1 (McMahon and Bradley, 1990; Thomas and Capecchi, 1990) or the Wnt receptor LRP6 (Pinson et al., 2000) result in the loss of the entire midbrain-hindbrain junction and Wnt-1 null mutants lack dopaminergic neurons (Danielian and McMahon, 1996) we investigated the possibility that Wnt-ligands increase the number of TH+ neurons in VM precursor cultures. Wnts are known to play a fundamental role in controlling cell fate decisions (Dorsky et al., 1998; Baker et al., 1999; Wilson et al., 2001; Garcia-Castro et al., 2002; Muroyama et al., 2002), cell proliferation (Chem and Walsh, 2002; Megason and McMahon 2002) and differentiation (Hall et al., 2000; Patapoutian and Reichardt, 2000; Krylova et al., 2002) in the nervous system, suggesting that multiple mechanisms can be involved in the regulation of dopaminergic neuron development in VM precursor cells.

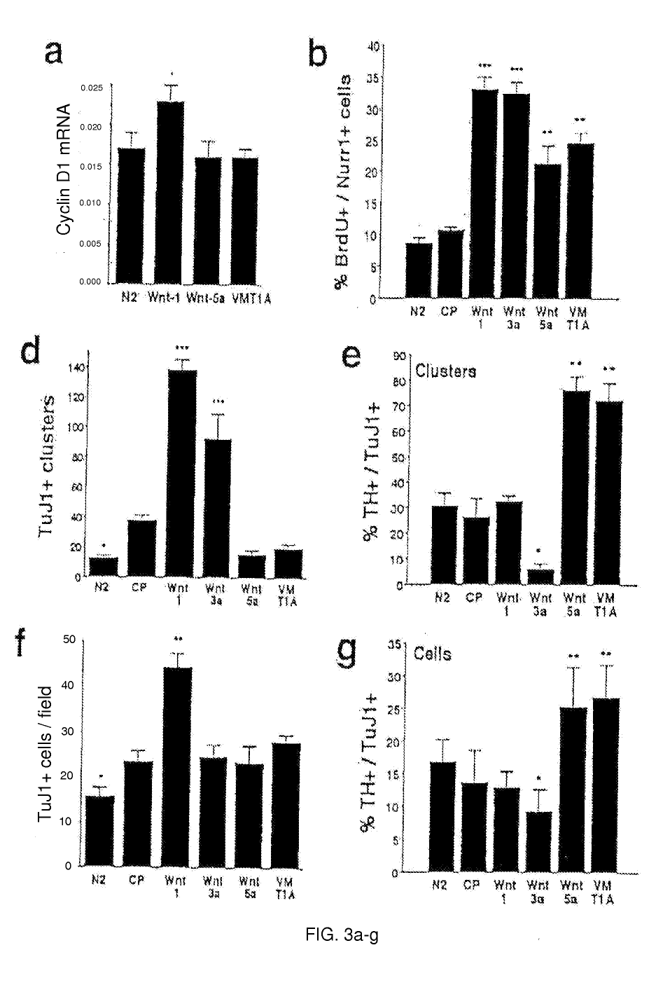

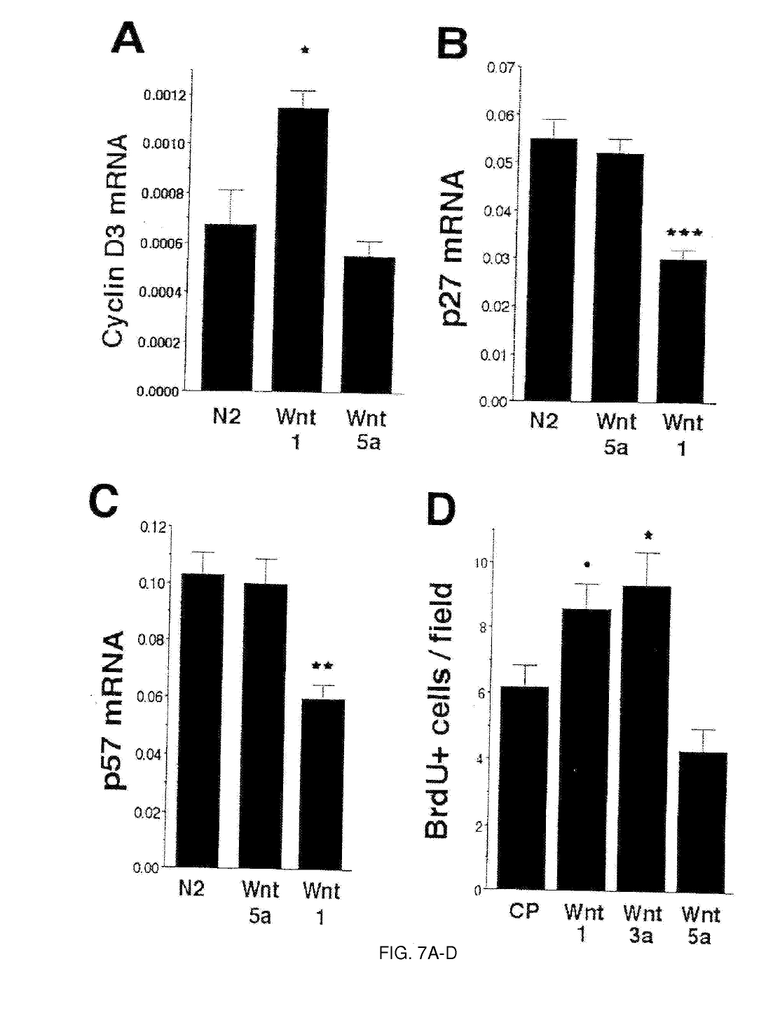

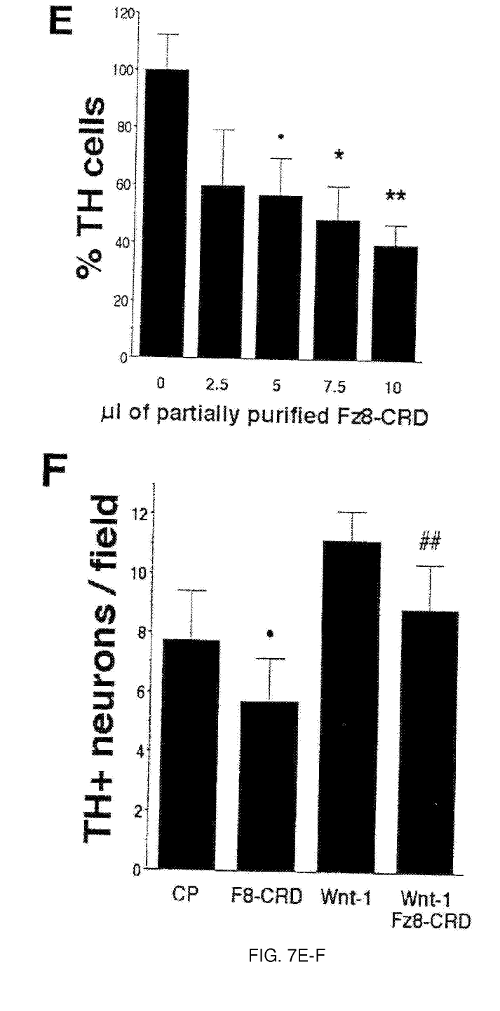

We examined by real time RT-PCR whether the expression of cyclin D1, a target of β-catenin (Tetsu and McCormick, 1999; Shtutman et al., 1999) that mediates cell cycle progression by Wnt-1 (Megason and McMahon 2002) but not by Wnt-5a (Kioussi et al., 2002), or other cyclins were regulated by Wnt-1, -5a and VM-T1A (FIG. 3A and FIG. 7A). While cyclin D2 mRNA was not affected by any of these treatments, cyclin D1 mRNA was upregulated by Wnt-1, but not by Wnt-5a or VM T1A, and cyclin D3 was upregulated by Wnt-1, but not by Wnt-5a. Our results provided an indication that Wnt-1 regulates cell cycle progression at a transcriptional level. Similarly, analysis of cell cycle inhibitors revealed that Wnt-1, but not Wnt-5a, downregulated p27 and p57 mRNA (FIGS. 7B and 7C), while neither affected p21 mRNA expression. Thus, our results suggest that Wnt-1, by regulating G1-S progression at a transcriptional level, may act to counterbalance cell cycle arrest induced by Nurr1 (Castro et al., 2001).

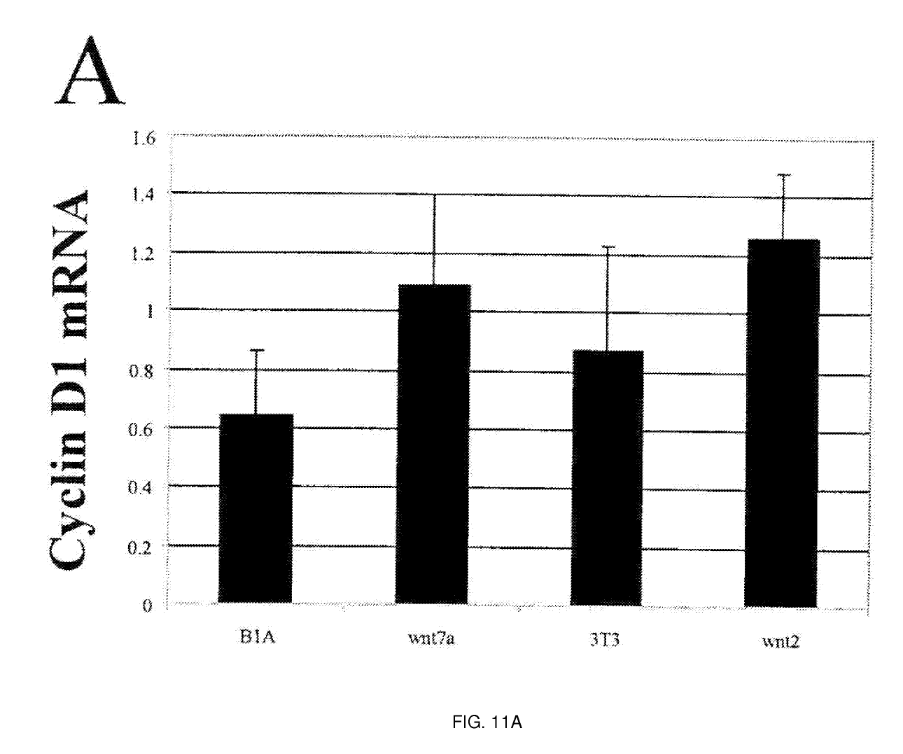

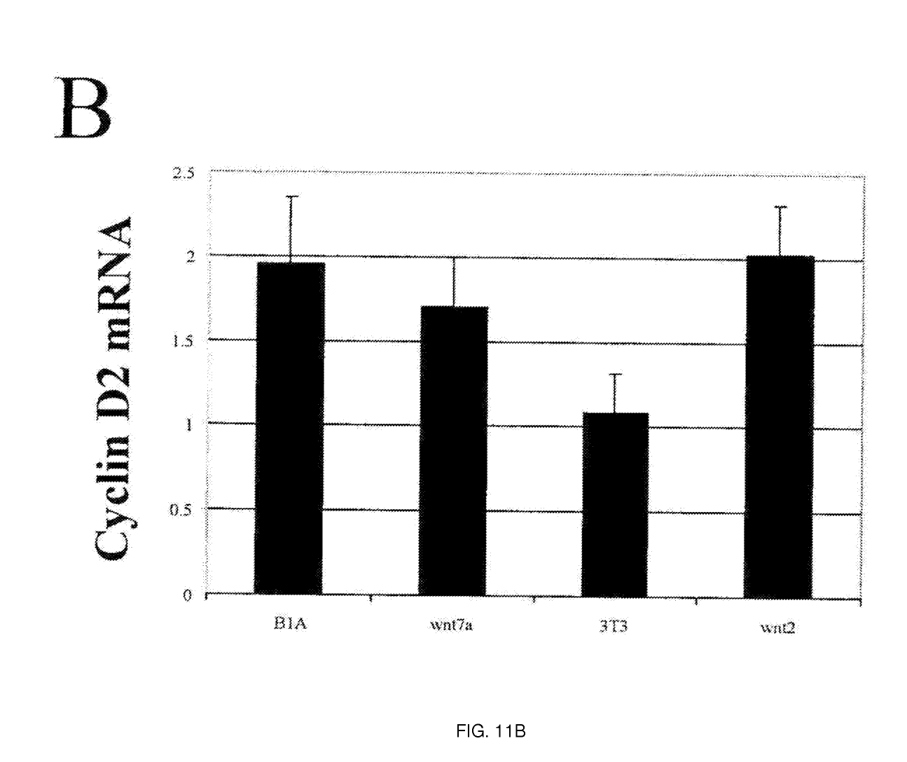

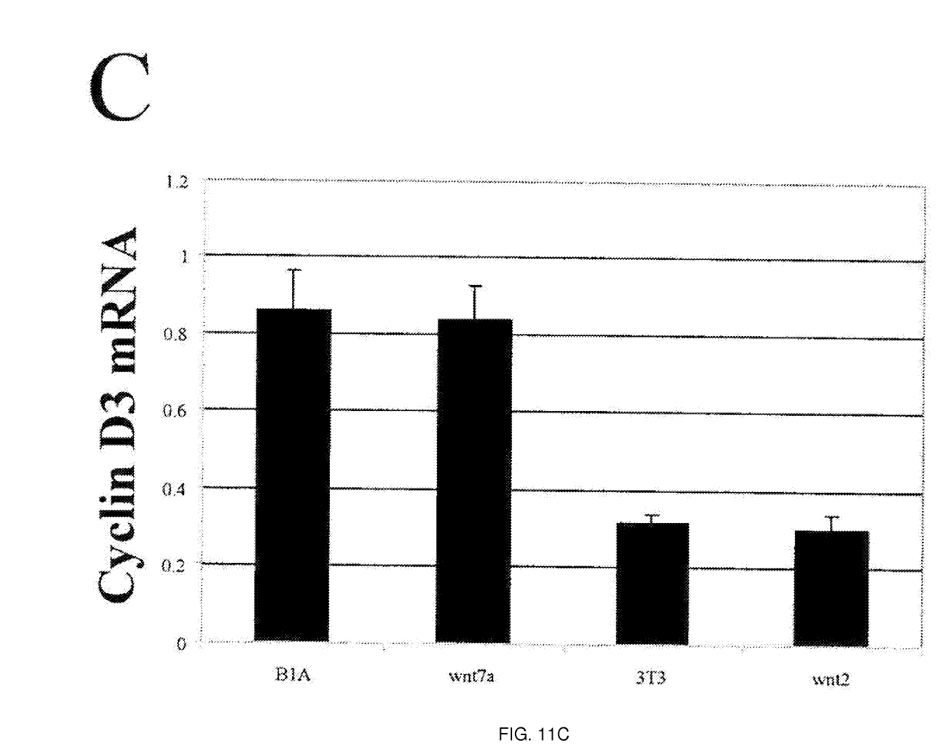

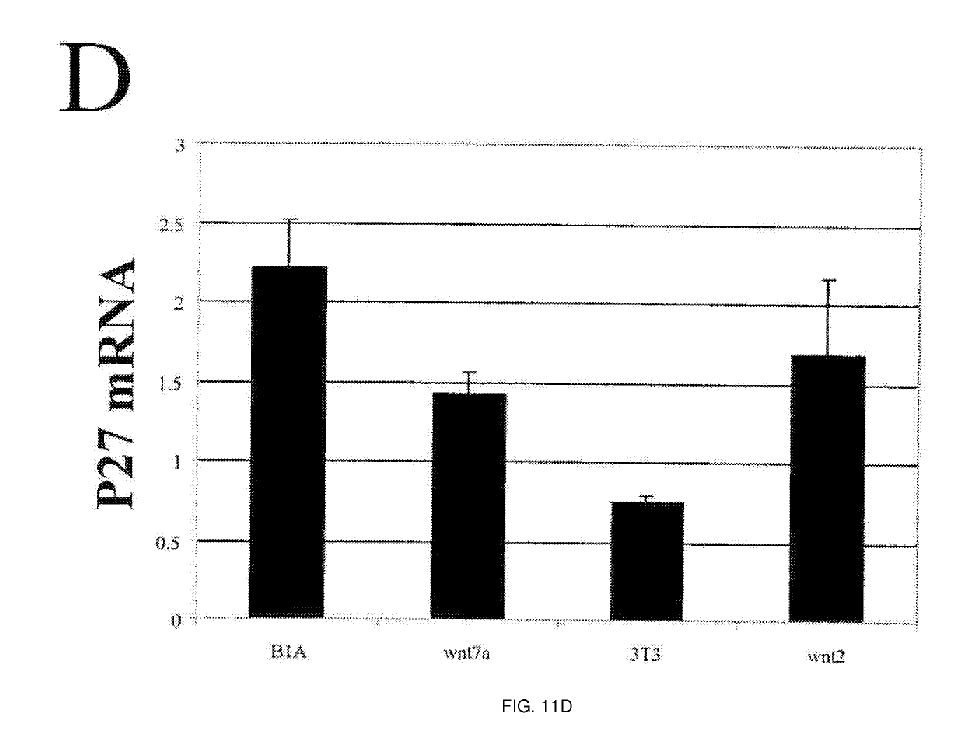

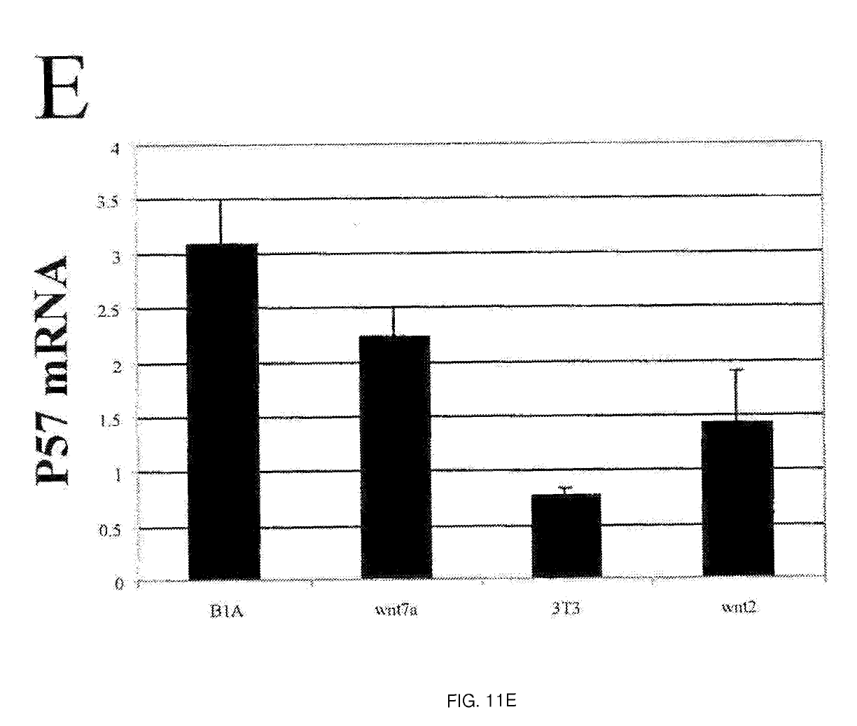

Wnt target genes have been identified in different biological systems and include many cell cycle regulators. Treatment with Wnt-2 and Wnt-7a resulted in distinct differences in the expression of key cell cycle regulators (FIG. 11). Surprisingly, upon treatment with Wnt-2, precursor cultures exhibited a slight upregulation in cyclin D1 mRNA levels (FIG. 11A). As expected, Wnt-7a increased cyclin D1 transcript levels (FIG. 11A), consistent with observations of increased levels of BrdU incorporation described herein. While cyclin D2 was not regulated by Wnt-7a, Wnt-2 treatment lead to increases in cyclin D2 mRNA levels (FIG. 11B). No regulation of cyclin D3 was observed with either Wnt-7a or Wnt-2a treatment (FIG. 11C). Interestingly, Wnt-7a treatment decreased the expression of the cell cycle inhibitors p27 and p57 (FIGS. 11D and 11E, respectively). These data indicate that Wnt-7a exerts its function on dopaminergic precursors by mechanism similar to that of Wnt-1, described herein. In contrast, increases in p27 and p57 mRNA levels were observed after treatment with Wnt-2 (FIGS. 11D and 11E, respectively). These data suggest that while Wnt-2 increases the number of TH+ neurons in precursors cultures, it does so by a mechanism separate from that of Wnt-5a and involving a negative regulation of the G1-S progression at a transcriptional level.

We examined whether Wnts regulated proliferation of DA precursor cells in VM cultures. Interestingly, we did not observe BrdU incorporation in newborn DA neurons (Nurr1+/TH+ cells), indicating that proliferating cells are not differentiating into TH+ cells during the 6 hour labelling period at the end of the experiment. We next examined whether Wnts regulated proliferation in VM precursor cultures and whether Nurr1-expressing neuronal precursors in the midbrain (Nurr1+/BrdU+ cells) were a potential target for Wnt ligands. BrdU incorporation in VM precursor cultures was increased by Wnt-1 and -3a, but not by Wnt-5a treatment (FIG. 7D), indicating that Wnt-1 and -3a may work as mitogens, promoting general precursor proliferation in the cultures. The effects of Wnt-1 and -3a were even greater (3-fold increase) when Nurr1 and BrdU double positive precursor cells were examined after 1 day in vitro. Partially purified Wnt-5a also increased mitosis in the Nurr1+ population but to a lesser extent (FIG. 3B). Double immunocytochemistry to Nurr1 and BrdU at 1 day in vitro demonstrated that Wnt-1 and -3a increased mitosis by 3 fold, while Wnt-5a and conditioned media from VM type 1 astrocytes, which express Wnt-5a and induce a dopaminergic phenotype in Nurr1-expressing precursors, increased mitosis by two fold in the Nurr1+ population (FIG. 3B). This finding, combined with the enhancement of cyclin D1 expression by Wnt-1 indicated that Wnt-1 mainly enhances the number of TH+ cells by regulating mitosis/proliferation. Surprisingly, Wnt-3a was as efficacious as Wnt-1 at enhancing mitosis in Nurr1 positive cells, but produced little or no increase in TH positive cell numbers, suggesting that increased mitosis is only one component of Wnt activity in the ventral mesencephalon. The clear increase in proliferation induced by Wnt-1 and Wnt-3 a correlates well with their ability to stabilize β-catenin (Shimizu et al., 1997), with the role of β-catenin in promoting cell cycle reentry in stem cells and neural precursors (Taipale and Beachy, 2001; Chem and Walsh, 2002)), and with the role of Wnt-3a in self-renewal of hematopoietic stem cells (Reya et al., 2003). Thus, the effects of Wnt-1 on precursor proliferation and the regulation of genes controlling G1-S progression indicate that Wnt-1 primarily enhances the number of TH+ cells by regulating mitosis/proliferation/self-renewal in precursor cells. However, Wnt-3a was as efficacious as Wnt-1 at enhancing mitosis in Nurr1+ cells but, unlike Wnt-1, had no effect or decreased the number of DA neurons, suggesting a specific role of Wnt-3a in self-renewal and/or maintaining the precursor population.

In experiments assessing the incorporation of BrdU (a marker of cells in S phase of mitosis), increases in the number of BrdU+ cells were also observed after treatment with Wnt-7a (FIG. 10C). In fact, Wnt-7a treatment resulted in BrdU+ increases similar to that of Wnt-1 described above. In contrast, a slight decrease in BrdU incorporation was noted following Wnt-2 treatment. These data indicate that while Wnt-7a increases the number of TH+ cells by expanding the precursor population, the effect of Wnt-2 occurs via a separate mechanism and involves reducing precursor proliferation.

We also examined whether the proliferative effects of Wnt-1 were specific and could be blocked by the cysteine-rich domain of Frizzled-8 (Fz8-CRD), a Wnt inhibitor (35,36). Partially purified CM from fibroblasts overexpressing Fz8-CRD, was added to control and Wnt-1 treated cultures and the number of TH+ neurons was examined (FIGS. 7D and 7E). Fz8-CRD decreased the number of TH+ neurons in both conditions, indicating that endogenous Wnts are necessary for the development of DA neurons in the cultures and that the effects of Wnts on TH+ cells are specific.

Thus in sum, all results indicated that the effects of Wnts were specific and that numbers of TH+ and proliferating cells in the VM are independently regulated by Wnts. Moreover, these results suggested that increased mitosis/proliferation/self-renewal was only one component of Wnt activity in the ventral mesencephalon.

We also asked whether the effects of Wnt ligands resulted in higher numbers of neurons and whether their effects were specific for dopaminergic neurons within or outside proliferating clusters. We found that Wnt-1 or -3 a increased the number of proliferating clusters containing TuJ1+ cells by 10-12 fold compared to control, Wnt-5a or VM T1A (FIG. 3d). Despite of the ability of both Wnt-1 and -3a to increase the number of proliferating clusters that can give rise to neurons, only Wnt-1 increased the total number of TuJ1+ neurons in the cultures (FIG. 3f). In addition, Wnt-1 did not increase the proportion of TH+ neurons (FIG. 3g) or VM clusters containing dopaminergic neurons (about 30% of the clusters) and Wnt-3a even decreased the proportion of dopaminergic neurons (FIG. 3g) and clusters to less than 10% (FIG. 3e). Our findings provided an indication that Wnt-1 and Wnt-3a increase the number of all VM neurons by increasing proliferation and the number of proliferating clusters. While Wnt-1 increased non-selectively the total number of VM neurons by a proliferative mechanism, Wnt-3a increased the proliferation of neuronal precursor cells growing as clusters and prevented their differentiation into TH+ cells, suggesting that they may play a role in the self-renewal of dopaminergic precursors. In contrast with Wnt-1 and -3a, Wnt-5a and VM T1A did not increase the number of TuJ1+ neurons (FIG. 3f) or proliferative clusters containing neurons (FIG. 3d), but instead selectively enriched for dopaminergic clusters and for dopaminergic neurons by more than two-fold (FIGS. 3e and 3g, respectively), providing indication that Wnt-5a and VM T1A could be involved in instructing or promoting the acquisition of a dopaminergic phenotype in VM cultures. The effects of Wnts on TuJ1+ neurons outside the clusters were very similar to those on neurons within clusters, except for Wnt-3a, that did not increase the number of TuJ1+ neurons (FIG. 3f), indicating that Wnt-3a regulates proliferation and/or self-renewal of precursors within clusters and by doing that decreases the proportion of neurons acquiring a dopaminergic phenotype (FIG. 3g).

Thus, our results suggested that Wnts independently and differentially regulate distinct functions in the VM: precursor proliferation and/or self-renewal, the number of neurons, and/or the proportion of neurons that acquire a dopaminergic phenotype. Moreover, despite the similar effects of Wnt-1, -5a and VM T1A on the number of dopaminergic neurons and cell clusters, our results provide an indication that Wnt-1 and Wnt-5a act on the dopaminergic cell lineage via two partially distinct mechanisms. While Wnt-1 increases proliferation of cells, but not the proportion of dopaminergic neurons, Wnt-5a and VM T1A work less on proliferation but instead increase the proportion of dopaminergic neurons within and outside proliferating clusters by an additional mechanism.

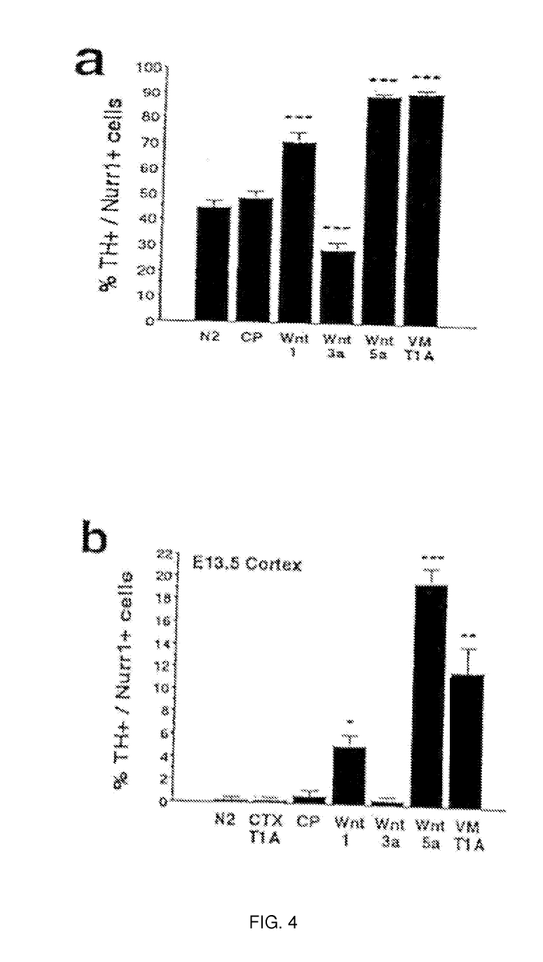

Provided that VM T1As are the source of a dopaminergic inductive signal (Wagner et al., 1999), we examined whether treatment with Wnts or VM T1A influenced the conversion of ventral mesencephalic Nurr1+ neuronal precursors (Nurr1+/TH− cells) into dopaminergic neurons (Nurr1+/TH+ cells). In control conditions, approximately 50% of all Nurr1 positive cells expressed TH, whereas Wnt-3a treatment decreases this population to 30%. In contrast, Wnt-1 increased the proportion of Nurr1+/TH+ to 70% and Wnt-5a or VM T1A increased this proportion to 90% (FIG. 4A). These results were very similar to those obtained with double ADH-2/TH immunostaining, since ADH2 also labeled dopaminergic precursors and neurons (Wagner et al., 1999). Thus, our data suggested a model in which Wnt-5a, unlike Wnt-1, predominantly increases the number of dopaminergic neurons by inducing a dopaminergic phenotype in Nurr1-expressing precursors. To test this model we examined whether Nurr1 positive/TH negative cells derived from brain structures other than VM could also be induced to acquire a dopaminergic phenotype by either Wnts or VM T1A. E13.5 primary cultures from the cerebral cortex, which are rich in Nurr1+ precursor cells, were exposed to various Wnt ligands or co-cultured with P1 VM or cortical astrocytes (as a control). Under Wnt-3a treatment or cortical T1A co-culture, virtually no Nurr1 positive cortical cells co-expressed TH. However, Wnt-1 Wnt-5a, or VM T1A treatment induced a significant increase in the proportion of Nurr1 positive cells that expressed TH (FIG. 4B). The most dramatic effect was induced by Wnt-5a, which, in only 3 days, increased the number of double Nurr1/TH+ cells by >40 fold compared to control and >4 fold compared to Wnt-1, an effect that can not be explained by proliferation alone. Moreover, the finding that Wnt-5a promotes the acquisition of a ventral dopaminergic phenotype correlates well with the previously reported role of Wnt-5a in promoting ventral fates via non-canonical Wnt signaling (Saneyoshi et al., 2002).

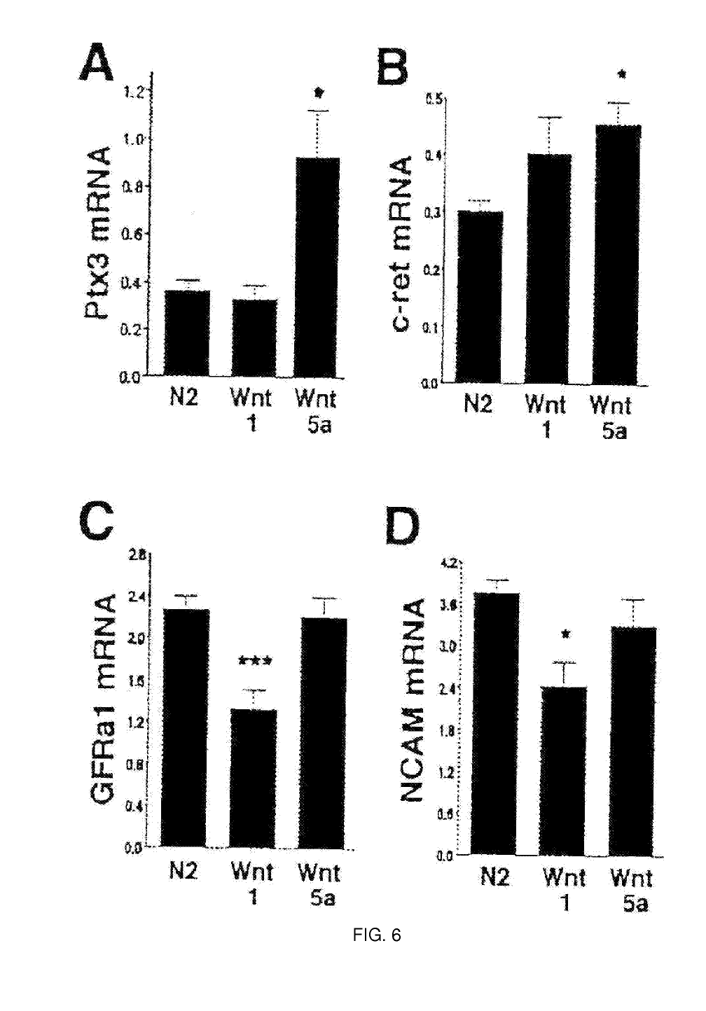

We also examined whether Wnt-5a treatment promoted the acquisition of a more differentiated midbrain DA phenotype in precursor cultures by regulating the expression of genes characteristic of midbrain dopaminergic neurons. We first examined the expression of the bicoid-related homeodomain gene Ptx3 (Smidt et al. 1997) by real time RT-PCR and found that Wnt-5a, but not Wnt-1 treatment, increased Ptx3 mRNA (FIG. 6A). This finding is very interesting because Ptx3 is required for the development of DA neurons (Van Den Munckhof P, et al. 2003; Nunes I., et al, 2003) and Ptx2, another homeodomain gene of the same family, is directly regulated by Wnt signaling (Kioussi C., et al. 2002; Baek S H et al., 2003). We next examined whether the expression of the proto-oncogene c-ret (Trupp M., et al., 1996) and the two other GDNF co-receptors, GFR□1 (Airaksinen and Saarma 2002) and NCAM (Paratcha et al., 2003), were also regulated by Wnts. Interestingly, both c-ret and NCAM are regulated by Wnt-signaling (Zheng S., et al. 1996; Conacci-Sorrell et al., 2002) and their ligand, GDNF, is required for the postnatal development of DA neurons (Granholm et al., 2000). We found that all the GDNF receptors analyzed were differentially regulated by Wnt-1 and Wnt-5a. While c-ret expression was upregulated by Wnt-5a, but not by Wnt-1 (FIG. 6B), the expression of GFR□1 and NCAM was maintained by Wnt-5a and repressed by Wnt-1 (FIGS. 6C and 6D). Thus, our results provide indication that Wnt-5a by increasing the expression of Ptx3 and GDNF receptors, two distinctive features of midbrain DA neurons, may be used to promote the acquisition of a DA phenotype in precursor cells and to enhance their differentiation and survival. Thus, Wnt-5a can increase the number of DA neurons.

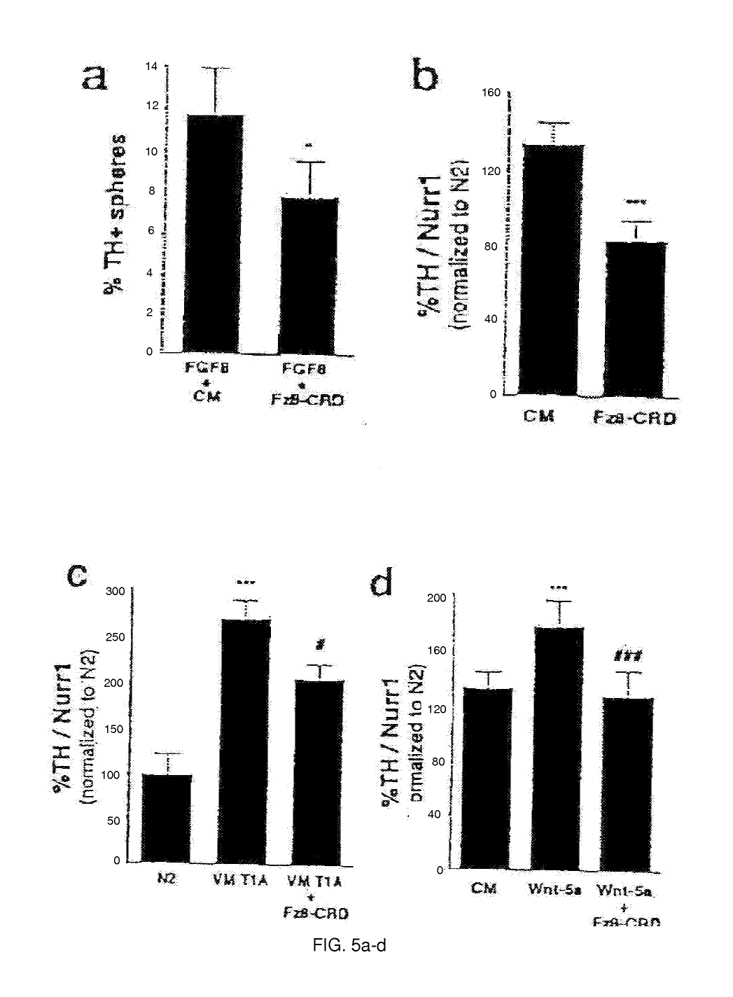

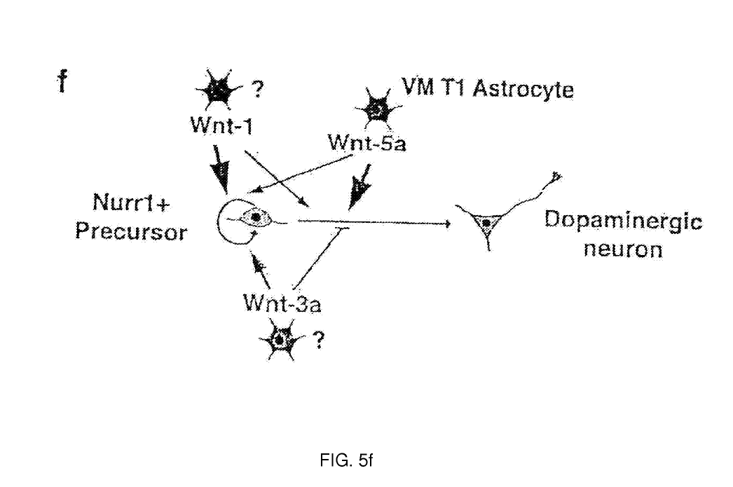

The finding that the biological activities of Wnt-5a were almost identical to those of VM T1A in all measured parameters (FIG. 1, FIG. 2, FIG. 3 and FIG. 4) and that VM T1A express Wnt-5a (FIG. 1F), provided indication that the effects of VM T1A might be in part due to astrocytic secretion of Wnts and promoted us to examine the function of Wnts in neural stem cell cultures. We therefore examined whether conditioned media from fibroblasts overexpressing the cysteine rich domain of Frizzled-8 (Fz8-CRD), which blocks the effects of Wnts (Hsieh et al., 1999), could block the activity of VM T1A or Wnt-5a on the induction of dopaminergic neurons. We found that addition of Fz8-CRD to Nurr1-expressing neural stem cells (Nurr1-c17.2-c42, Wagner et al., 1999) co-cultured with VM T1A partially blocked the induction of dopaminergic neurons. Moreover, Fz8-CRD reduced the number of dopaminergic neurons in VM neural stem cells grown as neurospheres expanded with FGF8 (FIG. 5a), a factor that in combination with Shh is known to induce dopaminergic neurons in preparations containing astrocytes, including the developing mouse brain (Ye et al., 1998) and neurospheres derived from Nurr1-expressing mouse embryonic stem cells (Kim et al., 2002). Similarly, co-treatment of E14.5 VM precursors with Fz8-CRD blocked the induction of dopaminergic neurons from Nurr1+ precursors in both untreated (FIG. 5b) and VM T1A (FIG. 5c) or Wnt-5a-treated cultures (FIG. 5d). Thus our results indicate that Wnts act in concert with other developmental signals and are in part required for the acquisition of a neuronal dopaminergic phenotype in precursor/neural stem cell cultures. Thus, our work suggests a model in which Wnts are essential regulators of two crucial and sequential aspects of neurogenesis in the ventral mesencephalon: precursor proliferation and acquisition of a dopaminergic phenotype (FIG. 5F). Thus, Wnts appear to regulate fate decisions of VM precursors. Wnt-1 promoted neurogenesis by increasing the proliferation of precursors and affected both DA and non-DA VM neurons. Wnt-3a promoted the proliferation or maintenance and/or self-renewal of Nurr1+ precursors and decreased the number of DA neurons. In contrast, Wnt-5a was a weak mitogen, increased the expression of Ptx3 and GDNF receptors, and efficiently promoted the acquisition of a DA phenotype in Nurr1-expressing precursors.

These results clearly show that dopaminergic precursors respond to Wnts in a very specific manner. Wnt-1, -3a, and -5a differentially regulated the development of midbrain DA neurons by partially overlapping mechanisms, that include promoting the proliferation of DA precursors (Wnt-1≧Wnt-3a>Wnt-5a), preventing their differentiation (Wnt-3a), extending neurogenesis (Wnt-1), and promoting the acquisition of midbrain dopaminergic phenotype (promoting differentiation of DA precursors into DA neurons) (Wnt-5a>Wnt-1). Similar to Wnt-1, Wnt-7a increased the number of TH+ cells by expanding the precursor population and promoting cell cycle at the G1-S transition. Wnt-2 increased the number of DA neurons by a different mechanism, involving a reduced proliferation of precursors and cell cycle arrest at G1.

With regard to the role of Wnts in regulating the acquisition of a midbrain dopaminergic phenotype in neural stem cells, we previously showed that astrocyte-derived signals play a decisive role in neurogenesis by inducing tissue specific neuronal phenotypes in Nurr1-expressing precursors, including that of midbrain dopaminergic neurons (Wagner et al., 1999, WO00/66713). Interestingly, a similar role of hippocampal astrocytes on adult neurogenesis was recently reported (Song et al., 2002). Herein we have demonstrated that astrocyte-derived signals include members of the Wnt family of ligands, that exert partially overlapping and distinct functions on Nurr1-expressing precursors. Wnt-1 promoted neurogenesis by increasing proliferation of neuronal precursors and affected all VM neurons. Wnt-3a promoted the proliferation and/or self-renewal of Nurr1+ precursors and decreased the number of dopaminergic neurons. In contrast, Wnt-5a was less efficient than Wnt-1 and -3a as a mitogen but was the most efficient at inducing a dopaminergic phenotype in Nurr1-expressing precursors. Moreover, the finding that Fz8 CRD blocked the induction of dopaminergic neurons not only in Nurr1-expressing precursors but also in FGF8-expanded neurospheres, indicates that Wnts are required for the induction of dopaminergic neurons and provides indication that the inductive effects of FGF8 may be mediated by Wnts. This possibility is in agreement with the known ability of FGF8 to induce both Wnt expression and organizer activity in the developing midbrain-hindbrain (for review see: Wurst and Bally-Cuif, 2001; and Rhinn and Brand 2001).

Our results identifying Wnts as key cell-extrinsic molecular players in the expansion of dopaminergic precursors/stem cells and the induction of midbrain dopaminergic neurons open the door to the efficient and large scale generation of stem cell-derived dopaminergic neurons for cell replacement in Parkinson's disease (Bjorklund and Lindvall 2000; Price and Williams 2001; Arenas 2002; Rossi and Cattaneo, 2002; Gottlieb et al., 2002). Thus, Wnt ligands and additional signals derived from ventral mesencephalic astrocytes or early glial cells may be used to induce ventral midbrain dopaminergic neurons. The present invention further provides for identification of additional components required for future implementation of efficient stem cell therapies.

### Treatment of Neurodegenerative Disease

Confirmation of the ability of neurons of the invention to treat neurodegenerative disease is obtained using an in vivo model of Parkinson's disease. Dopamine neurotoxins, 6-hydroxydopamine (6-OHDA) or MPTP are specifically taken up by neurons and lead to oxidative stress and loss of dopaminergic and noradrenergic neurons. Also, infusion of proteasome inhibitors, including lactacystin, or pesticides, including rotenone, are known to lead to the death of midbrain dopaminergic neurons and experimentally reproduce features of Parkinson's disease.

Cells expressing a nuclear receptor of the Nurr1 subfamily, e.g. Nurr1, that have been differentiated into dopaminergic neurons in vitro are surgically implanted into the substantia nigra and/or the striatum of 6-OHDA or MPTP or lactacystin or rotenone treated mice or other non-human animals. The ability of these cells to integrate and fully differentiate is evaluated by electrophysiological and/or morphological techniques in cells expressing reporter genes, such as LacZ and EGFP. Neurochemical techniques include measures of catecholamine contents and release. Morphological analysis may include studying the expression of marker genes characteristic of dopaminergic neurons, including tyrosine hydroxylase, dopamine transporter and dopamine receptors, Ptx3, Lmx1b and ADH-2, and the formation of synaptic contacts.

The ability of undifferentiated Nurr1+ cells to spontaneously differentiate in vivo toward the dopaminergic phenotype is assessed by intrastriatal or intranigral grafting of such cells into axotomized or 6-OHDA- or MPTPP- or lactacystin- or rotenone-treated animals. The dopaminerigc phenotype, differentiation and integration are detected as described above.

The ability of dopaminergic neurons derived from Nurr1+ cells grafted into the striatum and/or substantia nigra to rescue either motor asymmetries induced by unilateral toxin (e.g. 6-OHDA) treatment or motor abnormalities induced by systemic administration of MPP is confirmed by assessment of circling behaviour in apomorphine and amphetamine tests (Schwarting, R. K., et al., (1996) Progress in Neurobiology, 50(2-3), 275-331) and/or by performance in skilled paw usage in the staircase test, the stepping test or the cylinder test.

Host-derived endogenous stem, neural stem, progenitor or precursor or neural cells that express Nurr1 above basal levels in vivo, may be examined for their ability to differentiate into dopaminergic neurons after administration of a Wnt ligand in vivo. Analysis may include evaluation at a morphological, biochemical and behavioral levels, as described above. The ability of ventral mesencephalic astrocytes/glial cells or factors derived from them may be analysed as described above in conjunction with Wnt ligand administration.

### SUMMARY

The Wnts are a family of glycoproteins that regulate cell proliferation, self-renewal, fate decisions and differentiation. Our results show that β-catenin is expressed in Nurr1+ DA precursor cells and that Wnt-1, -3a and -5a are present at high levels in the VM and differentially regulated during development. Partially purified Wnts distinctively regulated VM development: Wnt-3a promoted the proliferation and/or self-renewal of Nurr1+ precursor cells but did not increase the number of TH+ neurons. Instead, Wnt-1 and -5a increased the number of rat midbrain DA neurons in precursor cultures by two distinct mechanisms. Wnt-1 predominantly increased the proliferation of Nurr1+ precursors, upregulated cyclin D1 and D3, and downregulated p27 and p57 mRNAs. In contrast, Wnt-5a primarily increased the proportion of Nurr1+ precursors that acquired a neuronal DA phenotype, which included the upregulation of Ptx3 and c-ret mRNA. Moreover, the soluble cysteine-rich domain of Frizzled 8 (a Wnt inhibitor) blocked the effects of Wnt-1 and Wnt-5a on proliferation and the acquisition of a DA phenotype in precursor cultures, and also blocked the effects of endogenous Wnts on the acquisition of a dopaminergic phenotype in Nurr1-expressing neural stem cells and FGF8-expanded VM neurosphere cultures.

The results described here reveal complex spatial and temporal expression patterns of the entire family of Wnt proteins in the developing rat midbrain. In addition to Wnt-1, Wnt-3a and Wnt-5a, this widescale expression analysis identified Wnt-2, Wnt-4, Wnt-7a and Wnt-7b as candidates with roles in the generation of dopaminergic neurons. The findings described in this work indicate that Wnt-7a works in a similar manner to Wnt-1, by promoting proliferation of dopaminergic precursors and allowing their differentiation into dopaminergic neurons. Our observations of increased BrdU incorporation, increased cyclin D1 expression, and decreased cdk inhibitor expression upon Wnt-7a treatment strongly support this. Also, this investigation revealed that the consequences of Wnt-2 treatment are: decreased levels of BrdU incorporation, increases in cyclin D2 expression, and large increases of expression of the cell cycle inhibitors p27 and p57. These data indicate that, compared with the other Wnts studied, Wnt-2 works via a new mechanism that favors cell cycle exit and the acquisition of a dopaminergic neuronal phenotype by Nurr1+ precursors. These findings highlight similar but distinct mechanisms of action by Wnts and indicate that Wnts are key regulators of proliferation, self-renewal and differentiation of stem/precursor cells into DA neurons. Moreover we found that all Wnts analysed exhibit unique activity profiles with regard to the mainteinance of precursor/stem cell proliferation and acquisition of a neuronal dopaminergic phenotype-suggesting that all members of the Wnt family may serve as attractive targets for the treatment of neuronal degeneration.

Thus, the methods described herein, which take advantage of the proliferation and differentiation potential of stem, neural stem, precursor, progenitor or neural cells, selector genes such as Nurr1, immature glial cells or astrocytes, and Wnts provide for the production of neurons of a desired neurochemical phenotype in the treatment of neurodegenerative diseases (e.g. as a source material for neuronal transplantation). The induction of midbrain dopaminergic neurons may be used in a cell replacement strategy to treat Parkinson's disease.

### Methods

### In Situ Hybridization (ISH)

Male and female wild type CD-1 mice (25-35 g, Charles River, Uppsala) were housed, bred and treated according to the guidelines of the European Community (86/609/EEC), the Society for Neuroscience (January 1985) and all experiments were approved by the local ethical committee. Mice from embryonic days 10.5 and 11.5 were removed and rapidly frozen in O.C.T at −70° C. Serial sagittal sections (14 μm thick) through the whole embryo were collected on glass microscope slides (StarFrost, KnittelGläser). ISH were performed on fresh frozen tissue with 35S labeled riboprobes as previously described (Trupp et al., 1997). In order to preserve mRNA levels sections were fixed for 15 minutes in ice-cooled 4% PFA and rinsed twice in PBS. Tissue was deproteinized in 0.2 M HCl for 12 min, acetylated with 0.25% acetic anhydride in 0.1 M triethanolamine for 20 min, and dehydrated in increasing concentrations of ethanol. Slides were incubated 16 h in a humidified chamber at 54° C. with 106 cpm of probe in 200 μl of hybridization cocktail. All the washes were performed at 62° C. First two washes of 15 min in 1×SSC, 30 min in 50% Formamide/0.5×SSC and 15 min in 1×SSC. Followed by 30 min RNase treatment (40 μg/ml) at 37° C. and two washes of 15 min in 1×SSC before dehydration in ethanol and air-drying. Chloroform was omitted and the dehydration time was reduced to 30 s-1 min. Subsequently the slides were dipped in NTB-2 photoemulsion (Eastman Kodak, Rochester, N.Y.) diluted 1:1 in water, exposed at 4° C. for 6-8 weeks, developed with D19 (Eastman Kodak), fixed with AL-4 (Agfa Gevaert, Kista, Sweden), and counterstained with thionin.

Immunohistochemistry was performed on 4% paraformaldehide (PFA) postfixed slides. Incubations were carried out at 4° C. overnight with mouse anti-β-catenin, 1:250 (BD Transduction Lab.) and rabbit anti-Nurr1, 1:200 (Santa Cruz Biotech.) in dilution buffer (phosphate-buffered saline, PBS, containing 1% bovine serum albumin, BSA, and 0.3% Triton-X 100). Following washes with 0.2% Tween-20/PBS, the sections were blocked for 30 min in dilution buffer, followed by a 2 hour incubation with a secondary antibody (Cy2 horse-anti-mouse IgG or horse-anti-rabbit IgG, or rhodamine horse-anti-mouse IgG, all from Jackson), 1:200.

### Real Time RT-PCR and Quantification of Gene Expression

Genbank cDNA sequences, including those for mouse and human Wnt 1, Wnt3a, Wnt5a, Frizzled 8, Ptx3, Cyclin D1 and Cyclin D2 and Tyrosine Hydroxylase, were used in Primer Express 1.0 (PE Applied Biosystems, Foster City, Calif., USA) and Primer 3 (http://www-genome.wi.mit.edu/cgi-bin/primer/primer3_www.cgi) for primer design. The following oligonucleotides were used:

Quantum RNA classical 18S internal standard (Ambion, Austin, USA);

mWnt1 forward

(SEQ ID NO: 1)

5′-CTTCGGCAAGATCGTCAACC-3′;
mWnt1 reverse

(SEQ ID NO: 2)

5′-GCGAAGATGAACGCTGTTTCT-3′;
mWnt3a forward

(SEQ ID NO: 3)

5′-GAACGCGACCTGGTCTACTACG-3′;
mWnt3a reverse

(SEQ ID NO: 4)

5′-GTTAGGTTCGCAGAAGTTGGGT-3′;
mWnt5a forward

(SEQ ID NO: 5)

5′-AATAACCCTGTTCAGATGTCA-3′;
mWnt5a reverse

(SEQ ID NO: 6)

5′-TACTGCATGTGGTCCTGATA-3′;
Tyrosine hydroxylase forward

(SEQ ID NO: 7)

5′-AGTACTTTGTGCGCTTCGAGGTG-3′;
Tyrosine hydroxylase reverse

(SEQ ID NO: 8)

5′-CTTGGGAACCAGGGAACCTTG-3′;
Fz8 forward

(SEQ ID NO: 9)

5′-TTGGAAGTGACCTCGCTCCTAG-3′;
Fz8 reverse

(SEQ ID NO: 10)

5′-GGTTGGGCATGTAAGTGTAGTTGT-3′;
Ptx3 forward

(SEQ ID NO: 11)

5′-AGGGTGGACTCCTACAGATTGG-3′;
Ptx3 reverse

(SEQ ID NO: 12)

5′-CCGATCCCAGATATTGAAGCC-3′;
Cyclin D1 forward

(SEQ ID NO: 13)

5′-ACCCTGACACCAATCTCCTCAAC-3′;
Cyclin D1 reverse

(SEQ ID NO: 14)

5′-GTAAGATACGGAGGGCGCACAG-3′;
Cyclin D2 forward

(SEQ ID NO: 15)

5′-ACTGATGTGGATTGTCTCAAAGCCT-3′;
Cyclin D2 reverse

(SEQ ID NO: 16)

5′-CGTTATGCTGCTCTTGACGGAA-3′;
C-ret forward

(SEQ ID NO: 17)

5′-ATGCACAATTACAGGCTGGTTCT-3′
C-ret reverse:

(SEQ ID NO: 18)

5′-GTCATTGACCAGGACTACTAGCTGC-3′
NCAM forward

(SEQ ID NO: 19)

5′-CACTTCGTGTTCAGGACTTCAGC-3′
NCAM reverse

(SEQ ID NO: 20)

5′-GGACGAAAATGACAATGAGGATG-3′
GFRa1 forward:

(SEQ ID NO: 21)

5′-GTCTGAGAATGAGATCCCCACAC-3′
GFRa1 reverse:

(SEQ ID NO: 22)

5′-ACACATTGGATTTCAGCTTCTGAG-3′.
Cyclin D3 forward:

(SEQ ID NO: 23)

5′-GGCTATGAACTACCTGGATCGCTA-3′
Cyclin D3 reverse:

(SEQ ID NO: 24)

5′-ACGGTACCTAGAAGCTGCAATTG-3′
p21 forward

(SEQ ID NO: 24)

5′-AGCAAAGTGTGCCGTTGTCTCT-3′
p21 reverse

(SEQ ID NO: 26)

5′-TCTCCGTGACGAAGTCAAAGTTC-3′
p27 forward

(SEQ ID NO: 27)

5′-TTAATTGGGTCTCAGGCAAACTCT-3′
p27 reverse

(SEQ ID NO: 28)

5′-CTAACCCAGCCTGATTGTCTGAC-3′
p57 forward

(SEQ ID NO: 29)

5′-GAGGACCAGAACCGCTGGGACTT-3′
p57 reverse

(SEQ ID NO: 30)

5′-ACTCGCTGTCCACCTCCATCCA-3′
GDNF forward:

(SEQ ID NO: 31)

5′-TTTCGATATTGTAGCGGTTCCTGT-3′
GDNF reverse:

(SEQ ID NO: 32)

5′-GCCTACCTTGTCACTTGTTAGCCT-3′
Wnt2 forward:

(SEQ ID NO: 33)

5′-AACGTCCCTCTCGGTGGAATC-3′
Wnt2 reverse:

(SEQ ID NO: 34)

5′-TGTACCACCATGAAGAGCTGACC-3′
Wnt2b/13 forward:

(SEQ ID NO: 35)

5′-CCACCCGGACTGATCTTGTCTACT-3′
Wnt2b/13 reverse:

(SEQ ID NO: 36)

5′-GGAACCTGAAGCCTTGTCCAA-3′
Wnt3 forward:

(SEQ ID NO: 37)

5′-CAGCGTAGCAGAAGGTGTGAAG-3′
Wnt3 reverse:

(SEQ ID NO: 38)

5′-ATGGCCAGGCTGTCATCTATG-3′
Wnt4 forward:

(SEQ ID NO: 39)

5′-GCTGTACCTGGCCAAGCTGTC-3′
Wnt4 reverse:

(SEQ ID NO: 40)

5′-TGGATCAGGCCTTTGAGTTTCTC-3′
Wnt5b forward:

(SEQ ID NO: 41)

5′-GCCGAGCTCTCATGAACCTACAG-3′
Wnt5b reverse:

(SEQ ID NO: 42)

5′-GGCGACATCAGCCATCTTATACAC-3′
Wnt6 forward:

(SEQ ID NO: 43)

5′-GCGGAGACGATGTGGACTTC-3′
Wnt6 reverse:

(SEQ ID NO: 44)

5′-ATGCACGGATATCTCCACGG-3′
Wnt7a forward:

(SEQ ID NO: 45)

5′-TGCGTGCCAGTCGAAACAAG-3′
Wnt7a reverse:

(SEQ ID NO: 46)

5′-GATATACACCAGGTCAGTGTCCATGG-3′
Wnt7b forward:

(SEQ ID NO: 47)

5′-GCCAACATCATCTGCAACAAGA-3′
Wnt7b reverse:

(SEQ ID NO: 48)

5′-CCGATCACAATGATGGCATC-3′
Wnt8a/8d forward:

(SEQ ID NO: 49)

5′-CAGCGACAACGTGGAGTTCG-3′
Wnt8a/8d reverse:

(SEQ ID NO: 50)

5′-CATCCTTCCCTTTCTCCAAACTG-3′
Wnt10a forward:

(SEQ ID NO: 51)

5′-CCACTCCGACCTGGTCTACTTTG-3′
Wnt10a reverse:

(SEQ ID NO: 52)

5′-TGCTGCTCTTATTGCACAGGC-3′
Wnt10b forward:

(SEQ ID NO: 53)

5′-ACGACATGGACTTCGGAGAGAAGT-3′
Wnt10b reverse:

(SEQ ID NO: 54)

5′-CATTCTCGCCTGGATGTCCC-3′
Wnt11 forward:

(SEQ ID NO: 55)

5′-CAAGTTTTCCGATGCTCCTATGAA-3′
Wnt11 reverse:

(SEQ ID NO: 56)

5′-TTGTGTAGACGCATCAGTTTATTGG-3′
Wnt15 forward:

(SEQ ID NO: 57)

5′-CTGTTCGTACCTGTTGGAAGCA-3′
Wnt15 reverse:

(SEQ ID NO: 58)

5′-CAGCCGTGTCATAGCGTAGCT-3′
Wnt16 forward:

(SEQ ID NO: 59)

5′-ACCCCCATTCTCAAGGATGACTT-3′
Wnt16 reverse:

(SEQ ID NO: 60)

5′-CAGTTTCTTGTTCTCCACGCAGTA-3′

Apart from 18S, all the remaining primers were purchased from Eurogentec, Seraing, Belgium and DNA Technology A/S, Aarhus, Denmark.

Total RNA was isolated from confluent T1A astrocyte cultures derived from ventral mesencephalon, dorsal mesencephalon and cortex of P1 rats and from tissue dissected from E10.5, E11.5, E13.5, E15.5 and P1 ventral and dorsal mesencephalon, and E14, E16, E18 and P1 cortex, using RNeasy extraction kit (Qiagen, Hilden, Germany). Total RNA was also extracted from E14.5 VM precursor cultures (7.5 million cells in 100 mm dishes) treated with wnts for 3 days in vitro (triplicate determination). For the reverse transcription, log of total RNA was initially treated with 1 unit RQ1 Rnase-free DNAse (Promega, Madison, USA) for 40 minutes. The DNAse was inactivated by the addition of 1 μl of EDTA 0.02M and incubation at 65° C. for 10 minutes. 0.5 μg random primers (Life Technologies, Grand Island, N.Y., USA) were then added, and the mixture was incubated at 70° C. for 10 minutes. Each sample was then equally divided in two tubes, a cDNA reaction tube and a negative control tube. A master mix containing 1× First-Strand Buffer (Life Technologies, Grand Island, N.Y., USA), 0.01M DTT (Life Technologies, Grand Island, N.Y., USA) and 0.5 mM dNTPS (Promega, Madison, USA) was then added to both cDNA and RT− tubes and incubated at 25° C. for 10 minutes, followed by a 2 minutes incubation at 42° C. 200 units of Superscript II reverse transcriptase (Life Technologies, Grand Island, N.Y., USA) were then added only to the cDNA tubes and all sample were incubated at 42° C. for 50 minutes. Superscript II was inactivated with an incubation for 10 minutes at 70° C. Both cDNA and RT− were then diluted 10 times, for further analysis.

Real-time PCR was performed in triplicates, with 1 μl 1:10 diluted cDNA and RT−, in a total volume of 25 μl. Each PCR reaction consisted on 1×PCR buffer (Life Technologies, Grand Island, N.Y., USA), 3 mM MgCl2 (Life Technologies, Grand Island, N.Y., USA), 0.2 mM dNTPs (Promega, Madison, USA), 0.3 μM each of the forward and reverse primers, 1 unit Platinum Taq DNA polymerase (Life Technologies, Grand Island, N.Y., USA) and 1×SYBR Green (Molecular Probes, Leiden, The Netherlands). The PCR was performed at 94° C. for 2 min and then for 35-40 cycles at 94° C. for 30 s, at 60° C. for 30-45 s, at 72° C. for 45-60 s and at 80° C. for 15 s (for SYBR Green detection) on the ABI PRISM 5700 Detection System (PE Applied Biosystems, Foster City, Calif., USA). Other annealing temperatures included 54° C. for Wnt 5a, 57° C. for p27, 62° C. for Cyclin D1, 61° C. for Cyclin D2 and 65° C. for p57. A melting curve was obtained for each PCR product after each run, in order to confirm that the SYBR Green signal corresponded to a unique and specific amplicon. The specificity of the PCR product was verified by sequencing.

Standard curves were generated in every 96-wells plate real time PCR run, using serial 3-fold dilutions of a reverse transcribed RNA or plasmid containing the sequence of interest for every probe. The resulting standard curve plots were then used to convert the Cts (number of PCR cycles needed for a given template to be amplified to an established fluorescence threshold) into arbitrary quantities of initial template of a given sample.

The expression levels were obtained by subtracting the RT− value for each sample from the corresponding RT+ value and then dividing that number by the value of the house-keeping gene, 18S, obtained for every sample in parallel assays.

Statistical analysis of the results was performed by one way ANOVA. Fisher's protected least significant difference was used post hoc to identify specific points at which the different developmental stages differed from the earliest, only when significant interactions occurred. Significance for all tests was assumed at the level of p<0.05 (*p<0.05; **p<0.001; ***p<0.000).

Each essay for a particular gene was repeated twice or thrice, in triplicate. 18S assays were run for each sample, at the beginning and once or twice at the middle of assays, to verify the integrity of the samples. The specificity of PCR primers was determined by BLAST run of the primer sequences. All the PCR products were run in a gel to verify the size of the amplicon. The specificity of the PCR product was determined by sequencing the amplicon in random samples.

### Precursor Cultures and Treatments

Ventral mesencephala and cerebral cortices from embryos E13.5-14.5 obtained from timed-mated Sprague-Dawley rats were dissected, mechanically dissociated and plated at a final density of 1×105 cells/cm2 on poly-D-lysine coated 12- or 24-well plates in a defined, serum-free medium (N2, consisting of a 1:1 mixture of F12 and DMEM containing insulin (10 ng/ml), transferrin (100 μg/ml), putrescine (100 μM), progesterone (20 nM), selenium (30 nM), glucose (6 mg/ml), and bovine serum albumin (1 mg/ml)). Purified Type 1 astrocytes were obtained from mixed glial cultures derived from the VM or CTX of P1 rats according to a standard protocol (Wagner et al., 1999). After shaking and replating into 12-well plates, astrocytes were grown to confluency in 15% fetal bovine serum-containing media and changed to N2 medium, at which time freshly dissected VM or CTX cells were plated on top of the astrocytes at a density of 1×105 cells/cm2. All factors were added once, at the initiation of culture, with the exception of 5-bromodeoxyuridine (BrdU), which was added 4-6 hours prior to fixation. Cultures were maintained in a humidified 5% CO2, 95% air incubator at 37° C. and fixed after given time periods with 4% paraformaldehyde for 45 minutes prior to immunocytochemical analysis.

### Neurosphere Cultures

Ventral mesencephala from E13.5 rat embryos were dissected and pooled together, ressuspended in N2 serum-free media, mechanically dissociated and plated at a final density of 100-125×103 cells/cm2, in 24-well plates (BD Bioscience, Erembodegem, Belgium) previously coated with poly-D-lysin (Sigma, Stockholm, Sweden). The cells were expanded in the presence of 20 ng/ml FGF8b (R&D Systems, Minneapolis, USA) and 8 μg/ml heparin (Sigma, Stockholm, Sweden) and after 7-10 days replated at high sphere density in N2, supplemented by the partially purified conditioned medias, and Fz8CRD at 125-250 μg/ml of protein. The neurospheres were then differentiated for 5-7 days. Fixation and immunocytochemistry analysis were performed as previously elsewhere.

### Wnt Conditioned Media Preparation, Characterization and Purification

B1A fibroblast lines stably overexpressing hemagglutanin-tagged Wnt-1a, 3a, or 5a (Shimizu H., 1997) were grown in standard complete media (DMEM+10% FBS) supplemented with 100 ug/ml G-418. For collection of conditioned media, cells were replated at low density in complete media and allowed to reach 50-75% confluency, at which point cells were washed and media was replaced with serum-free N2 (with 10 μM sodium butyrate) for 24 hours. Conditioned media from sister flasks was then harvested, pooled as lots and stored at −80° C. for up to 2 months. When compared to fresh CM, no loss of activity was observed when using this collection and storage routine. T1A CM was harvested using a similar procedure, with the exception that media was collected after 3 days in vitro.

For concentration, individual lots of CM were thawed at room temperature, divided into 80 ml aliquots, loaded onto Centricon-Plus 80 columns (Millipore) and concentrated via centrifugation according to the manufacturers instructions. Following concentration, aliquots were re-pooled and frozen at −80 C after a sample was taken for determination of protein content and Western blot analysis. In brief, 20 ug of protein was loaded onto a 10% polyacrylamide mini-gel and run under denaturing conditions at 150 V for approximately 30 minutes. After dry electroblot transfer onto PVDF membranes (Hybond P, Amersham Pharmacia Biotech, Upsalla, Sweden) and pre-incubation for 30 minutes in a blocking buffer consisting of 3% bovine serum albumin in Tris-Buffered Saline with X % Triton-X 100 (TBST), blots were incubated with mouse anti-hemagglutinin (Babco) diluted 1:1000 in TBST overnight at 4 C. After washing, blots were incubated with alkaline phosphatase-coupled goat anti-mouse IgG (Santa Cruz) diluted 1:1000 in TBST for 1 hour at room temperature. Blots were visualized using the Amersham ECF reagent and blue fluorescence quantitated using a Molecular Devices Storm 840 phosphoimager.

### Fz8CRD Conditioned Media Preparation, Characterization and Purification

B1A fibroblast lines were cotransfected with the mFz8CRD-IgG/pRK5 and the pIRES puro 2 (Clontech, BD Bioscience, Erembodegem, Belgium) plasmids, using Lipofectamine Plus reagent (Invitrogen, Lidingo, Sweden) according to the instruction of the manufacturer. Upon selection for puromycin resistance (1 μg/ml Sigma, Stockholm, Sweden), clones were isolated and expanded. A clone overexpressing Fz8CRD 700× (real time RT-PCR analysis) was identified. Harvesting and partial purification of Fz8CRD conditioned media were performed as described previously. In all the blocking experiments performed, the Fz8CRD and B1A control partial purified conditioned medias were used at a final concentration of 125-250 μg/ml.

### Immunocytochemical Analysis

Fixed cultures were incubated with one of the following antibodies, diluted appropriately in phosphate-buffered saline (PBS) containing 1% bovine serum albumin and 0.3% Triton-X 100: mouse anti-BrdU, 1:50 (DAKO, Denmark), mouse anti-β-tubulin, Type III (TuJ1), 1:250 (Sigma), mouse anti-TH, 1:1000 (Incstar, USA), rabbit anti-TH, 1:250 (PelFreeze); rabbit anti-Nurr1, 1:2000 (gift from Dr. T. Perlmann, Sweden), rabbit anti-Nurr1, 1:1000 (Santa Cruz, USA) or mouse anti-Nurr1, 1:250 (BD Transduction Laboratories, USA), rabbit anti Adh2 1:4000 (gift from Dr. R. Lindahl, South Carolina). Incubations were either carried out at 4° C. overnight, or at room temperature for 1 hour. Both processes yielded similar results. After washing, cultures were incubated for 1-3 hr with appropriate secondary antibodies (biotinylated 1:500; CY2-, FITC-, or rhodamine-coupled horse-anti-mouse IgG 1:100; or goat-anti-rabbit IgG 1:100; all from Vector, USA), in the same dilution buffer. Brightfield immunostaining was visualized with the Vector Laboratory ABC immunoperoxidase kit, using either NovaRed (red), or AEC (red), SG or 3-3′ dimaminobenzidine tetrahydrochloride (DAB 0.5 mg/ml)/nickel chloride (1.6 mg/ml) (gray/black), or VIP (violet) substrates. Double-staining was performed by sequential single staining as described. The order of staining was only critical for BrdU-double labeling, in which case the BrdU procedure was always performed second. Control experiments in which either of the primary or secondary antibodies used were deleted demonstrated little to no cross-reactivity between the antibody pairs used. Photos were acquired with a Zeiss Axioplan 100M microscope and collected with a Hamamatsu camera C4742-95 (with the QED imaging software).

### Data Analysis and Statistics

Only clearly stained cells were counted as positive cells. Spheres were considered positive if they contained one or more positive cell. Quantitative immunocytochemical data on individual cells represent means and standard errors of counts obtained by a blinded observer from 10-20 non-overlapping fields (cells) or in the entire well (clusters), in each of 3-4 wells per condition from 3-4 independent experiments, unless stated otherwise. For each variable, initial statistical comparisons were performed by a global ANOVA, with multiple factors of dose, time point, treatment and/or region; if significant interactions between treatment and any other variable existed, data were further divided into separate times, doses or regions. Fisher's protected least significant difference was used post hoc to identify specific points at which the treatments differed from controls (or each other) only when significant interactions between factor treatment and other variables occurred. Significance for all tests was assumed at the level of p<0.05. To simplify presentation in the Results section, the outcome of individual statistical analyses, as well as the nature of the analyses performed, is presented within individual figure legends.

### REFERENCES

Airaksinen M S, Saarma M. (2002) Nat Rev Neurosci. 3, 383-394
Akerud et al. J. Neurosci. 21, 8108-18 (2001).
Arenas E. Brain Res Bull. 6, 795-808 (2002).
Baek S H et al., (2003) Proc Natl Acad Sci USA. 100, 3245-3250
Bain et al., 1995, Dev. Biol., 168, 342-357
Baker et al. Genes Dev 1999 Dec. 1; 13(23):3149-59
Bjorklund et al., 2002, Proc Natl Acad Sci USA. February 19, 99(4): 2344-9
Bjorklund A, Lindvall O. Nat. Neurosci. 2000 June; 3(6):537-44.
Bradley R S, Brown A M. Mol Cell Biol 15(8):4616-22 (1995).
Bradley R S, Brown A M. EMBO J. 9(5):1569-75 (1990).
Brustle et al., 1999, Science 285, 754-756.
Capela and Temple, 2002, Neuron 35, 865-875.
Castillo (1997) Genomics, 41, 250-257
Castillo et al. Mol Cell Neurosci 1998 May; 11(1-2):36-46
Castro D S, et al., (2001) J Biol. Chem. 276, 43277-43284.
Chem A, Walsh C A. Science. 2002 Jul. 19; 297(5580):365-9.
Conacci-Sorrell M E, Ben-Yedidia T, Shtutman M, Feinstein E, Einat P, Ben-Ze'ev A. (2002) Genes Dev. 16, 2058-2072.
Danielian P S, McMahon A P. (1996) Nature 383, 332-334
Dann C E, et al., (2001) Nature 412, 86-90.
Deacon et al., 1998, Exp. Neurol., 149, 28-41.
Dorsky et al. Nature 1998 Nov. 26; 396(6709):370-3
Foster et al. Int J Dev Neurosci 1988; 6(4):367-86
Garcia-Castro et al. Science. 2002 Aug. 2; 297(5582):848-51
Gossen, et al., (1995) Science, 268, 1766-1769
Gottlieb D I. Annu Rev Neurosci. 2002; 25:381-407.
Granholm A C, Reyland M, Albeck D, Sanders L, Gerhardt G, Hoernig G, Shen L, Westphal H, Hoffer B. (2000) J Neurosci. 20, 3182-3190.
Hall A C, Lucas F R, Salinas P C. (2000) Cell 100, 525-535.
Hartmann C, Tabin C J. Cell 2001 Feb. 9; 104(3):341-51
Houart et al. Neuron. 2002 Jul. 18; 35(2):255.
Hsieh et al. Proc Natl Acad Sci USA. 96(7):3546-51 (1999).
Kiecker et al. Development 2001 November; 128(21):4189-201
Kim et al. Nature 2002 Jul. 4; 418(6893):50-6
Kioussi C., et al. (2002) Cell 111, 673-685, 38
Kispert et al. Development 1998 November; 125(21):4225-34
Krylova O, et al., (2002) Neuron 35, 1043-1056.
Law, et al., (1992) Mol Endocrinol 6, 2129-2135
Lee et al., 2000, Nature Biotech., 18, 675-679.
Lindsay 2002; Cur. Op. Pharmacol., 2: 587-594.
Marvin et al. Genes Dev 2001 Feb. 1; 15(3):316-27.
Martinez Arias et al., 1999, Curr. Op. Genetics and dev. 9, 447-454.
Mata de Urquiza et al., Science 2000 Dec. 15; 290: 2140-2144.
McMahon A P, Bradley A. Cell 1990 Sep. 21; 62(6):1073-85
Megason S G, McMahon A P. Development 129(9):2087-98 (2002).
Muroyama et al. Genes Dev. 16(5):548-53 (2002).
Nordstrom et al. Nat Neurosci 2002 June; 5(6):525-32
Nunes I., et al, Proc Natl Acad Sci USA. 100, 4245-4250 (2003)
Okabe et al., 1996, Mech. Dev., 59, 89-102.
Ohmachi et al., 2000; Biochem. Biophys. Res. Comm. 277, 355-360.
Oishi I, et al., 2003, Genes Cells. 2003 July; 8(7):645-54.
Pandur et al. Nature. 2002 Aug. 8; 418(6898):636-41.
Paratcha G., Ledda F., Ibanez C. F. (2003) Cell 113, in press.
Parr et al. Development 119(1):247-61 (1993).
Patapoutian A, Reichardt L F. (2000) Curr Opin Neurobiol. 10, 392-399.
Pinson K I. et al., (2000) Nature 407, 535-538.
Price J. & Williams B P. Current Opinion in Neurobiology 11, 564-567 (2001).
Reubinoff et al., 2000, Nat. Biotech., 18, 399-404.
Reubinoff et al., 2001, Nat. Biotech., 19, 1134-1140.
Reya T, et al., (2003) Nature 423, 409-414.
Rhinn M, Brand M. Curr Opin Neurobiol 2001 February; 11(1):34-42
Ross et al. Science 2000 Aug. 11; 289(5481):950-3
Rossi F. & Cattaneo E. Nat Rev Neurosci. 2002 May; 3(5):401-9.
Saucedo-Cardenas et al. Proc Natl Acad Sci USA 1998 Mar. 31; 95(7):4013-8
Saneyoshi et al. Nature 417(6886):295-9 (2002).
Schuldiner et al., 2000, PNAS. USA 97, 11307-11312.
Schuldiner et al., 2001; Brain Res. 913, 201-5.
Shimizu et al. Cell Growth Differ 1997 December; 8(12):1349-58
Shtutman et al. Proc Natl Acad Sci USA 96(10):5522-7 (1999).
Schneider et al. Genes Dev 2001 Feb. 1; 15(3):304-15
Smidt et al. Proc Natl Acad Sci USA. 94(24):13305-10 (1997).
Smidt et al., Nat. Neurosci. 3, 337-341 (2000).
Song et al. Nature 2002 May 2; 417(6884):39-44
Taipale J, Beachy P A. Nature. 2001 May 17; 411(6835):349-54.
Thomas K R, Capecchi M R. Nature 1990 Aug. 30; 346(6287):847-50
Tetsu et al. Nature 398(6726):422-6 (1999).
Tropepe. et al., 2001, Neuron 30, 65-78.
Trupp M et al., (1996). Nature 381, 785-789
Tzahor E, Lassar A B. Genes Dev 2001 Feb. 1; 15(3):255-60
Van Den Munckhof P, et al. 2003 Development 130, 2535-2542
Wagner et al. Nat Biotechnol 1999 July; 17(7):653-9
Wilson et al. Nature 2001 May 17; 411(6835):325-30
Wurst W, Bally-Cuif L. Nat Rev Neurosci 2001 February; 2(2):99-108
Xing, et al., (1997) Mol Brain Res 47, 251-261
Ye et al. Cell 93(5):755-66 (1998).
Yoshikawa S., et al., Nature. 2003 Apr. 10; 422(6932):583-8.
Zetterstrom et al. Science 1997 Apr. 11; 276(5310):248-50
Zhang et al., 2001, Nat. Biotech., 19, 1129-1133.
Zheng S, Ramachandran B, Haigh J R, Palos T P, Steger K, Howard B D. (1996).

1. A method of inducing or promoting dopaminergic neuronal development by enhancing proliferation, self-renewal, dopaminergic induction, survival, differentiation and/or maturation in a neural stem, progenitor or precursor cell, or other stem or neural cell, the method comprising:
expressing a nuclear receptor of the Nurr1 subfamily above basal levels within the cell, and
treating the cell with a Wnt ligand,
thereby producing or enhancing proliferation, self-renewal, survival and/or dopaminergic induction, differentiation, survival or acquisition of a neuronal dopaminergic phenotype.

2. A method according to claim 1 wherein the nuclear receptor is Nurr1.

3. A method according to claim 1 wherein the nuclear receptor is Nor1 or NGFI-B.

4. A method according to claim 1 comprising expressing Nurr1 above basal levels by transforming a cell with Nurr1 DNA or introducing into the cell Nurr1 RNA.

5. A method according to claim 1 comprising expressing Nurr1 above basal levels by introducing Nurr1 protein into the cell or by preserving Nurr1 protein in the cell.

6. A method according to claim 1 wherein the Wnt ligand is selected from the group consisting of a Wnt-1 ligand, a Wnt-5a ligand, a Wnt-3a ligand, a Wnt-2 ligand, a Wnt-4 ligand, a Wnt-7a ligand and a Wnt-7b ligand.

7. A method according to claim 1 wherein said neural stem, progenitor or precursor cell or other stem cell or neuronal cell is treated with Wnt ligands other than Wnt-1 or Wnt-5a or an additional Wnt ligand.

8. A method according to claim 1 wherein the neural stem, progenitor or precursor cell or other stem or neural cell is mitotic and/or capable of self-renewal when it is treated with the Wnt ligand.

9. A method according to claim 1 wherein said neural stem, progenitor or precursor cell or other stem or neural cell is additionally contacted with at least one of (i) a member of the FGF family of growth factors and (ii) at least one substance selected from the group consisting of a retinoid or retinoid derivative, an activator of the retinoid X receptor (RXR), a repressor of the retinoid acid receptor (RAR), 9-cis retinal, DHA, SR11237, or LG849.

10. A method according to claim 1 wherein the neural stem, progenitor or precursor cell or other stem or neural cell is treated with bFGF and/or EGF and/or FGF-8 and/or LIF and/or Shh prior to or simultaneously with treating the cell with a Wnt ligand.

11. A method according to claim 1 wherein the neural stem, progenitor or precursor cell or other stem or neural cell is grown in the presence of antioxidants, ascorbic acid, low oxygen tension or a hypoxia-induced factor.

12. A method according to claim 1 wherein the neural stem, progenitor or precursor cell or other stem or neural cell grows and/or differentiates in the presence of ventral mesencephalic astrocytes or early glial cells.

13. A method according to claim 1 wherein the Wnt ligand is added to an in vitro culture containing the cell.

14. A method according to claim 13 wherein Wnt ligand is produced by expression from a cell co-cultured with the neural stem, progenitor or precursor cell, or other stem or neural cell, which co-cultured cell is a cell other than a type 1 astrocyte or early glial cell or is a host cell transformed with nucleic acid encoding the Wnt ligand or a cell containing introduced Wnt protein.

15. A method according to claim 14 wherein the co-cultured cell other than a type 1 astrocyte or early glial cell or host cell is another stem, neural stem, progenitor, precursor or neural cell.

16. A method according to claim 13 wherein the neural stem, progenitor or precursor cell, or other stem or neural cell, is engineered to express the Wnt ligand from encoding nucleic acid.

17. A method according to claim 1 comprising further co-culturing the neural stem, progenitor or precursor cell, or other stem or neural cell, with an early glial cell, or a Type 1 astrocyte optionally of the ventral mesencephalon.

18. A method according to claim 17 wherein the Type 1 astrocyte is immortalized or is of an astrocyte cell line of a region other than the ventral mesencephalon.

19. A method according to claim 1, comprising additionally contacting the neural stem, progenitor or precursor cell, or other stem or neural cell with a negative selection agent that selects against non-dopaminergic neurons.

20. A method according to claim 1 further comprising formulating a neuron into a composition comprising one or more additional components, said composition optionally including a pharmaceutically acceptable excipient.

21. A method according to claim 20 further comprising administering the composition to an individual.

22. A method according to claim 21 wherein the neuron is implanted into the brain of the individual.

23. A method according to claim 20 wherein the individual has Parkinson's disease, a parkinsonian syndrome, neuronal loss or a neurodegenerative disease.

24. A method according to claim 1 further comprising:
(i) treating a dopaminergic neuron with a toxin for said dopaminergic neuron;
(ii) separating the dopaminergic neuron from the toxin;
(iii) bringing the treated dopaminergic neuron into contact with a test agent or test agents;
(iv) determining the ability of the dopaminergic neuron to recover from the toxin;
(v) comparing said ability of the dopaminergic neuron to recover from the toxin with the ability of a dopaminergic neuron to recover from the toxin in the absence of contact with the test agent or test agents.

25. A method according to claim 1 further comprising:
(i) treating a dopaminergic neuron with a toxin for the dopaminergic neuron in the presence of a test agent or test agents;
(ii) determining the ability of the dopaminergic neuron to tolerate the toxin;
(iii) comparing said ability of the dopaminergic neuron to tolerate the toxin with the ability of a dopaminergic neuron to tolerate the toxin in the absence of contact with the test agent or test agents.

26. A method of obtaining a factor or factors which, either alone or in combination, enhance proliferation, self-renewal, survival and/or dopaminergic development, induction, differentiation, or maturation in a neural stem, progenitor or precursor cell, or other stem or neural cell expressing Nurr1 above basal levels, the method comprising:
(a) treating a neural stem progenitor or precursor cell, or other stem or neural cell expressing Nurr1 above basal levels with a Wnt ligand in the presence and absence of one or more test substances; and
(b) determining proliferation, self-renewal, survival and/or dopaminergic development, induction, differentiation, or maturation of the cell and comparing the extent of the proliferation, self-renewal, survival and/or dopaminergic development, induction, differentiation or maturation in the presence and absence of the test substance or substances, whereby said factor or factors is obtained.

27. A method according to claim 26, wherein the cell is treated with the Wnt ligand by introduction of Wnt ligand protein into the cell.

28. A method according to claim 26 wherein the neural stem, progenitor or precursor cell, or other stem or neural cell is treated with the Wnt ligand by co-culturing with a cell which is a cell other than a type 1 astrocyte or early glial cell or is a host cell transformed with nucleic acid encoding the Wnt ligand or a cell containing introduced Wnt protein, and said method optionally further comprises co-culturing the neural stem, progenitor or precursor cell, or other stem or neural cell with an early glial cell or a Type 1 astrocyte optionally of the ventral mesencephalon.


SEQUENCE LISTING (60 sequences)

SEQ ID NO: 1 - DNA, 20 bp, Artificial Sequence
  primer
cttcggcaagatcgtcaacc

SEQ ID NO: 2 - DNA, 21 bp, Artificial Sequence
  primer
gcgaagatgaacgctgtttct

SEQ ID NO: 3 - DNA, 22 bp, Artificial Sequence
  primer
gaacgcgacctggtctactacg

SEQ ID NO: 4 - DNA, 22 bp, Artificial Sequence
  primer
gttaggttcgcagaagttgggt

SEQ ID NO: 5 - DNA, 21 bp, Artificial Sequence
  primer
aataaccctgttcagatgtca

SEQ ID NO: 6 - DNA, 20 bp, Artificial Sequence
  primer
tactgcatgtggtcctgata

SEQ ID NO: 7 - DNA, 23 bp, Artificial Sequence
  primer
agtactttgtgcgcttcgaggtg

SEQ ID NO: 8 - DNA, 21 bp, Artificial Sequence
  primer
cttgggaaccagggaaccttg

SEQ ID NO: 9 - DNA, 22 bp, Artificial Sequence
  primer
ttggaagtgacctcgctcctag

SEQ ID NO: 10 - DNA, 24 bp, Artificial Sequence
  primer
ggttgggcatgtaagtgtagttgt

SEQ ID NO: 11 - DNA, 22 bp, Artificial Sequence
  primer
agggtggactcctacagattgg

SEQ ID NO: 12 - DNA, 21 bp, Artificial Sequence
  primer
ccgatcccagatattgaagcc

SEQ ID NO: 13 - DNA, 23 bp, Artificial Sequence
  primer
accctgacaccaatctcctcaac

SEQ ID NO: 14 - DNA, 22 bp, Artificial Sequence
  primer
gtaagatacggagggcgcacag

SEQ ID NO: 15 - DNA, 25 bp, Artificial Sequence
  primer
actgatgtggattgtctcaaagcct

SEQ ID NO: 16 - DNA, 22 bp, Artificial Sequence
  primer
cgttatgctgctcttgacggaa

SEQ ID NO: 17 - DNA, 23 bp, Artificial Sequence
  primer
atgcacaattacaggctggttct

SEQ ID NO: 18 - DNA, 25 bp, Artificial Sequence
  primer
gtcattgaccaggactactagctgc

SEQ ID NO: 19 - DNA, 23 bp, Artificial Sequence
  primer
cacttcgtgttcaggacttcagc

SEQ ID NO: 20 - DNA, 23 bp, Artificial Sequence
  primer
ggacgaaaatgacaatgaggatg

SEQ ID NO: 21 - DNA, 23 bp, Artificial Sequence
  primer
gtctgagaatgagatccccacac

SEQ ID NO: 22 - DNA, 24 bp, Artificial Sequence
  primer
acacattggatttcagcttctgag

SEQ ID NO: 23 - DNA, 24 bp, Artificial Sequence
  primer
ggctatgaactacctggatcgcta

SEQ ID NO: 24 - DNA, 23 bp, Artificial Sequence
  primer
acggtacctagaagctgcaattg

SEQ ID NO: 25 - DNA, 22 bp, Artificial Sequence
  primer
agcaaagtgtgccgttgtctct

SEQ ID NO: 26 - DNA, 23 bp, Artificial Sequence
  primer
tctccgtgacgaagtcaaagttc

SEQ ID NO: 27 - DNA, 24 bp, Artificial Sequence
  primer
ttaattgggtctcaggcaaactct

SEQ ID NO: 28 - DNA, 23 bp, Artificial Sequence
  primer
ctaacccagcctgattgtctgac

SEQ ID NO: 29 - DNA, 23 bp, Artificial Sequence
  primer
gaggaccagaaccgctgggactt

SEQ ID NO: 30 - DNA, 22 bp, Artificial Sequence
  primer
actcgctgtccacctccatcca

SEQ ID NO: 31 - DNA, 24 bp, Artificial Sequence
  primer
tttcgatattgtagcggttcctgt

SEQ ID NO: 32 - DNA, 24 bp, Artificial Sequence
  primer
gcctaccttgtcacttgttagcct

SEQ ID NO: 33 - DNA, 21 bp, Artificial Sequence
  primer
aacgtccctctcggtggaatc

SEQ ID NO: 34 - DNA, 23 bp, Artificial Sequence
  primer
tgtaccaccatgaagagctgacc

SEQ ID NO: 35 - DNA, 24 bp, Artificial Sequence
  primer
ccacccggactgatcttgtctact

SEQ ID NO: 36 - DNA, 21 bp, Artificial Sequence
  primer
ggaacctgaagccttgtccaa

SEQ ID NO: 37 - DNA, 22 bp, Artificial Sequence
  primer
cagcgtagcagaaggtgtgaag

SEQ ID NO: 38 - DNA, 21 bp, Artificial Sequence
  primer
atggccaggctgtcatctatg

SEQ ID NO: 39 - DNA, 21 bp, Artificial Sequence
  primer
gctgtacctggccaagctgtc

SEQ ID NO: 40 - DNA, 23 bp, Artificial Sequence
  primer
tggatcaggcctttgagtttctc

SEQ ID NO: 41 - DNA, 23 bp, Artificial Sequence
  primer
gccgagctctcatgaacctacag

SEQ ID NO: 42 - DNA, 24 bp, Artificial Sequence
  primer
ggcgacatcagccatcttatacac

SEQ ID NO: 43 - DNA, 20 bp, Artificial Sequence
  primer
gcggagacgatgtggacttc

SEQ ID NO: 44 - DNA, 20 bp, Artificial Sequence
  primer
atgcacggatatctccacgg

SEQ ID NO: 45 - DNA, 20 bp, Artificial Sequence
  primer
tgcgtgccagtcgaaacaag

SEQ ID NO: 46 - DNA, 26 bp, Artificial Sequence
  primer
gatatacaccaggtcagtgtccatgg

SEQ ID NO: 47 - DNA, 22 bp, Artificial Sequence
  primer
gccaacatcatctgcaacaaga

SEQ ID NO: 48 - DNA, 20 bp, Artificial Sequence
  primer
ccgatcacaatgatggcatc

SEQ ID NO: 49 - DNA, 20 bp, Artificial Sequence
  primer
cagcgacaacgtggagttcg

SEQ ID NO: 50 - DNA, 23 bp, Artificial Sequence
  primer
catccttccctttctccaaactg

SEQ ID NO: 51 - DNA, 23 bp, Artificial Sequence
  primer
ccactccgacctggtctactttg

SEQ ID NO: 52 - DNA, 21 bp, Artificial Sequence
  primer
tgctgctcttattgcacaggc

SEQ ID NO: 53 - DNA, 24 bp, Artificial Sequence
  primer
acgacatggacttcggagagaagt

SEQ ID NO: 54 - DNA, 20 bp, Artificial Sequence
  primer
cattctcgcctggatgtccc

SEQ ID NO: 55 - DNA, 24 bp, Artificial Sequence
  primer
caagttttccgatgctcctatgaa

SEQ ID NO: 56 - DNA, 25 bp, Artificial Sequence
  primer
ttgtgtagacgcatcagtttattgg

SEQ ID NO: 57 - DNA, 22 bp, Artificial Sequence
  primer
ctgttcgtacctgttggaagca

SEQ ID NO: 58 - DNA, 21 bp, Artificial Sequence
  primer
cagccgtgtcatagcgtagct

SEQ ID NO: 59 - DNA, 23 bp, Artificial Sequence
  primer
acccccattctcaaggatgactt

SEQ ID NO: 60 - DNA, 24 bp, Artificial Sequence
  primer
cagtttcttgttctccacgcagta